(19) United States
(12) Patent Application Publication
(10) Pub. No.: US 2016/0166683 A1
(43) Pub. Date: Jun. 16, 2016

(54) ADJUVANT IMMUNOTHERAPY FOR THE TREATMENT CANCER, OF CLINICAL MANIFESTATIONS ASSOCIATED WITH THE DISEASES LIKE CACHEXIA AND CORRECTION OF ADVERSE EFFECTS OF DRUGS SUCH AS IMMUNOSUPPRESSION, SECUNDARY CACHEXIA, NEUTROPENIA AND LYMPHOPENIA, COMPRISING THE ASSOCIATION OR COMBINATION OF A BIOLOGICAL RESPONSE MODIFIER SPECIALLY SELECTED AND OTHER SUBSTANCES WITH ANTINEOPLASTIC ACTION AND/OR OTHER TREATMENTS

(71) Applicant: lseu da Silva NUNES, Campinas-Sao Paulo (BR)

(72) Inventor: lseu da Silva NUNES, Campinas-Sao Paulo (BR)

(21) Appl. No.: 15/049,904

(22) Filed: Feb. 22, 2016

Related U.S. Application Data
(63) Continuation-in-part of application No. 13/516,628, filed on Jun. 15, 2012, now Pat. No. 9,295,682.

(51) Int. Cl. A61K 39/39; A61K 31/7068; A61K 31/513; A61K 31/517; A61K 31/4745; A61K 31/519; A61K 31/167; A61K 33/24; A61K 31/28

(52) U.S. Cl. CPC A61K 39/39; A61K 33/24; A61K 31/7068; A61K 31/513; A61K 31/28; A61K 31/4745; A61K 31/519; A61K 31/167; A61K 31/517

(57) ABSTRACT

A compound for use in a method of treatment of cancer, including precancerous lesions, and adverse events caused by the disease or anti-cancer agents and treatments, such as cancer cachexia, lymphopenia, neutropenia, febrile neutropenia includes in combination an immunomodulatory and at least one anti-cancer agent or treatment suitable for treating the disease. The immunomodulator is a proteic aggregate of ammonium and magnesium phospholinoleate-palmitoleate anhydride. The anti-cancer agent or treatment suitable for treating the disease provides synergistic effects without additional toxicity when used with the immunomodulatory. The anti-cancer treatment is selected from the following group: surgical procedures, transplantation of bone marrow cells, systemic and localized radiotherapy, and combinations thereof.

### RELATED APPLICATIONS

This application is a Continuation-in-Part of co-pending application Ser. No. 13/516,628, filed on Jun. 15, 2012, which is a U.S. National Stage of International Application No. PCT/BR09/00424 filed on Dec. 15, 2009, for which priority is claimed under 35 U.S.C. §120; the entire contents of all of which are hereby incorporated by reference.

### THE PRIOR ART

Clinical Problems Related to Cancer—Main Difficulties and Problems Related to Treatments—State of the Art

Despite significant advances in medicine, many types of tumors still show disappointing response to all types and modalities of treatments available, as for example, small cell lung cancer, stomach cancer, pancreatic cancer, cervical cancer, invasive melanoma, cancer of the adrenal cortex and soft tissue sarcomas. Additionally, many types of tumors or cancerous cells possess or develop mechanism that makes them resistant to chemotherapy and radiotherapy.

Regarding cancer prevention or preventive treatment, there is an urgent need to develop new treatments, because few effective treatments are available in the state of the art.

Preventive therapeutic action in cancer is based on the information that several types of cancers such as malignant skin tumors, those that affect epithelial tissue lining of internal organs, cervical cancer, among others, begin in small populations of normal cells in which aggressive external agents such as solar radiation, chemical products and some types of viruses can induce or promote cell alterations considered precancerous lesions, that over time have the ability to become tumors of high grade malignity and great mortality.

Therefore, it is essential that these precancerous lesions are eliminated or reversed early, which requires the availability of new and better drugs and treatments, for use alone or in combination with the existing therapies.

Regarding the treatment of cancer using surgical techniques, one of the main difficulties faced in the total elimination of the tumors is that, at the time of diagnosis, a considerable number of cancer patients already have micro metastases or metastases in other parts of the body, far from the original location of the tumor, and in most cases these metastases cannot be detected during the surgical procedure.

Aiming to increase the chances of successful surgery, in the state of the art in medicine, new strategies have been developed in view of the ever-present possibility of occurrence of micro metastases or metastases, although these are undetectable at the time of the surgical procedure.

In such cases, the availability of non-surgical treatment modalities, such as the previous, concomitant or subsequent administration of chemotherapy drugs, had a great impact on cancer treatment, greatly improving the chances of patients undergoing surgery.

As an example of these strategies, neoadjuvant or induction chemotherapy can be cited, which consists in treating the patient with drugs, usually cytotoxic drugs, before surgery.

In some cases, this type of chemotherapy is aimed at reducing the size of the tumor or the extent of surgery required and is often used to evaluate the patient's response to chemotherapy drugs and, thus, assist in the elaboration of the best postoperative treatment strategies and protocols.

Another modality of treatment is adjuvant chemotherapy, which consists in administering chemotherapy drugs to the patient, alone or in association with other drugs, after surgery, with the purpose of blocking the formation or development of tumors close or far from the main location, or finally, to reduce the tumor growth rate, in the cases of micro metastases or metastases refractory to treatment.

Finally, depending on tumor staging, that is, on the phase of tumor growth, its location or the presence of metastatic disease, cytotoxic medications may cease to be effective. Therefore, combinations of drugs with different mechanisms of action are increasingly used in the state of the art to improve the success of therapies and treatments.

These combinations, which are called chemotherapy regimens or protocols that involve the use of various compounds, combined or associated, depending on patient and disease state, are common practices in the state of the art.

Despite the advances in the state of the art, thanks to the development of new drugs and treatments for cancer, there are still many difficulties and problems associated to drugs and treatments that often have a negative impact on their effectiveness.

However, while it is undeniable that the greater availability of new drugs intended for use in chronic systemic diseases, such as cancer, as well as the adoption of treatment protocol involving the combination of various drugs for the treatment of one patient, has led to a significant increase in the response rate of response to treatment and to a higher survival rate for patients, on the other hand, there are many serious adverse side effects associated to the use of these drugs and/or combinations of drugs.

Examples include adverse side effects associated to the use of cytotoxic drugs, because they have the ability to interfere with multiple cellular metabolic processes and, thus, indiscriminately affect tumor and normal cells, causing severe problems, such as immunosuppression, hair loss or alopecia, damage to the mucous membranes in the digestive tract, to name the most common adverse side effects.

Some cytotoxic antibiotics used in cancer treatment, e.g., doxorubicin, mitoxantrone and mitomycin, act on DNA replication and on the integrity of cellular DNA, being generally toxic and adversely affecting both cancerous and healthy cells.

Other widely used compounds, such as vinca alkaloids and taxanes, act on various essential cell structural components and, thus, damage healthy cells too.

In general, it can be said that most cytotoxic drugs used in the treatment of s cancer, damage healthy cells too, including bone marrow cells, cells of the gastrointestinal tract and other important cell structures, this being one of the major problems regarding the use of the aforementioned drugs.

Severe adverse effects associated to their use may interfere with the effectiveness of cancer treatment itself, because they can lead to reduction of the dose (dosage or period of administration) or even to treatment discontinuation.

Finally, radiotherapy, one of the most common treatments for some types of cancer, besides causing damage to tissues and organs, can also affect the immune response, contributing to the decrease in the number of circulating lymphocytes, mainly B and T lymphocytes. For an example of state of the art research, please see: Helman, Principle of Radiation Therapy, in Cancer Principles and Practice of Oncology, Vol. 1, 3nd Ed.—DeVita et al. Eds, J.B. Lippincott Co. Publ.—247-275 (1989).

Unfortunately, hematological and immunological abnormalities such as anemia and neutropenia, respectively, which can be associated to the use of chemotherapy compounds and/or radiation, are common and serious complications for patients undergoing treatment.

Neutropenia associated to the use of several drugs and other non-drug treatments such as radiotherapy is a serious undesirable event for patients with chronic systemic diseases, such as cancer, because besides providing suitable conditions for the occurrence of infectious events caused by opportunistic pathogens, they have also a negative impact on the function of components and other cell elements of the immune system of the host, which are also very important in fighting cancer cells.

In general, the same problems associated to the use of compounds or drugs such as poor therapeutic response, adverse side effects, immunosuppression, neutropenia and anemia are common occurrences for the other treatments available in the state of the art for cancer, such as radiotherapy.

Another serious clinical complication associated to cancer is cachexia—a Greek word that means poor condition—also known as malnutrition-cachexia syndrome and which can be further complicated when drugs and other treatments are added.

Finally, in cancer, the quality of life of patients is significantly affected either by the disease or by clinical conditions associated with the disease, such as cachexia and/or by the worsening of clinical and psychological conditions associated to adverse effects related to the disease and treatments.

Occurrence of tumor resistance, poor therapeutic response in many cases, accelerated physical deterioration, metastatic processes, deterioration of quality of life, among others, are adverse consequences for the whole body affected by the primary disease and/or aggravated by the use of existing treatments and drugs, and which are major challenges to be faced in the state of the art. Thus, new inventions and improvements are needed in the treatment of chronic diseases, such as cancer.

The present invention, as will be widely explained and demonstrated, shall contribute innovatively to the resolution of these problems in various ways.

And in order to demonstrate and facilitate understanding of the state of the art, novelty and usefulness of the present invention, several aspects and important clinical conditions associated cancer, the treatments available in the state of the art and the main difficulties and/or limitations associated to their use, as well as relevant information to the understanding of the usefulness of the invention for the treatment of cancer and associated clinical conditions and for the solution or minimization of the problems and limitations of the existing treatments are detailed here.

Main Clinical Complications Associated to Cancer-Cachexia or Anorexia-Cachexia Syndrome—Drugs and Treatments—State of the Art

Malnutrition and cachexia are morbid conditions often presented by patients with serious chronic systemic pathologies, such as cancer.

In the common form of malnutrition, the body turns to its own fat reserves, sparing the muscle tissue, whereas in cachexia, there is equal mobilization of these reserves and quick loss of muscle and fat. For examples of state of the art, please see: Body J J. The syndrome of anorexia-cachexia. Curr Opin. Oncol. 1999; 11(4):255-60) e Moley J F Aamodt R, Rumble W, Kaye W, Norton J A. Body cell mass in cancer bearing and anorexia patients. J Parenter. Ent. Nutr. (1987; 11:219-22).

Cachexia in cancer patients is a complex syndrome clinically characterized by generalized depletion of muscle and fat tissues, causing progressive and involuntary weight loss, anemia, astenia, negative nitrogen balance, immune dysfunction and metabolic changes.

Because of its relation with anorexia, the term Cachexia-anorexia syndrome or CAS, (abbreviation) has been used frequently in the state of the art to describe clinical pictures of malnutrition and cachexia associated to cancer and other serious systemic diseases.

Anorexia-Cachexia—ACS—Causes

The origin of cachexia or the anorexia-cachexia syndrome (ACS) in cancer patients is multifactorial in the state of the art.

These factors include: the increased energy (glucose) uptake by tumor, the release of factors that act on satiation, being able to reduce food intake and the number of cytokines produced by the host reportedly cause metabolic abnormalities of the referred syndrome. Examples in the state of the art include Bosaeus I, Daneryd P, Lundholm K. Dietary intake, resting energy expenditure, weight loss and survival in cancer patients. J Nutr. 2002; 132(11 Suppl):3465S-3466S.

Cachexia in cancer patients can be classified into primary or secondary: Primary cachexia that is related to the metabolic effects of cancer associated to inflammatory changes. Its consequence is the progressive and often severe depletion of visceral protein, skeletal muscle and fat tissue.

Secondary cachexia is due to decreased nutrient intake and absorption, which can be caused by cancerous obstruction of the gastrointestinal tract, and also as a result of surgical, chemotherapy, radiotherapy treatments and their combinations.

Both conditions may occur concomitantly in the same patient over the course of the disease.

Anorexia Syndrome—Cachexia and Cancer Diagnosis

Malnutrition associated to weight loss induced by cancer is one of the most commonly used factors in patient assessment and for establishing a prognosis of clinical evolution.

Many studies in the state of the art indicate that patients with marked weight loss (cachexia) also show very poor therapeutic response to chemotherapy (CT), and toxicity of drugs used in patient treatment is increased.

Example of state of the art research: Body J J—The syndrome of anorexia-cachexia. Curr Opin. Oncol. 1999; 11(4):255-60) The degree of cancer cachexia is inversely correlated with the survival time of the patient and involves deterioration in quality of life and poor prognosis for patients.

### Clinical Expressions of Cachexia and Consequences for the Treatment of Patients

Cachectic patients may have higher susceptibility to infectious agents, postoperative complications, reduced tolerance to cancer treatment and also pronounced drowsiness and prostration. Due to loss of muscle mass, these patients are at greater risk of developing decubitus ulcers, edema of lower limbs and intense paleness. Cachexia is present in more than 80% of advanced cancer patients (Inui A. Cancer anorexia-cachexia syndrome: current issues in research and management. CA Cancer J Clin. 2002; 52:72-91), (Body J J. The syndrome of anorexia-cachexia. Curr Opin Oncol. 1999; 11(4): 255-6), being responsible for a decrease of around 60% of body weight compared to the ideal weight (Argilés J M, Moore-Carrasco R, Fuster G, Busquets S, Lopez-Soriano F J. Cancer cachexia: the molecular mechanisms. Int J Biochem Cell Biol. 2003; 35(4): 405-9).

Cachexia is the main cause of death in more than 20% of cancer patients: (Inui A. Cancer anorexia-cachexia syndrome: current issues in research and management. CA Cancer J Clin. 2002; 52:72-91).

Anorexia-Cachexia Syndtome—Assesment Criteria

Body weight is the most commonly used nutritional parameter in patient assessment. An example of the state of the art research follows: Inui A. Cancer anorexia-cachexia syndrome: current issues in research and management. CA Cancer J Clin. 2002; 52:72-91. (e 2).

A sudden loss of weight of around 10% of body weight is considered the parameter used to establish the beginning of the anorexia-cachexia syndrome (ACS). For a state of the art example please see: Inui A. Cancer anorexia-cachexia syndrome: current issues in research and management. CA Cancer J Clin. 2002; 52:72-91.

Treatment of the Anorexia-Cachexia Syndrome—State of the Art

For explanatory purposes, without intending to limit the scope of the present invention, a brief summary of state of the art treatments available for patients with cancer-related malnutrition and cachexia and other chronic systemic diseases are presented here.

The main interventions and treatments available for the treatment of anorexia-cachexia syndrome (ACS) in chronic systemic diseases such as cancer include nutritional and pharmacological therapies that use several types of drugs, either alone or combined.

State of Art—Anorexia—Cachexia Treatment—Nutritional Therapy

The nutritional treatment or therapy of anorexia-cachexia states is performed with the use of special nutrients, such as polyunsaturated fatty acids, eicosapentaenoic acid (EPA) and docosahexanenoic acid (DHA), the amino acids glutamine and arginine and the nucleotides used in formulations and dietary supplements.

Polyunsatured Fatty Acids (PUFAs): Eicosapentaenoic acid (EPA) and Docosahexaenoic Acid (DHA).

Eicosapentaenoic acid (EPA)[IUPAC: Acid (5Z,8Z,11Z,14Z,17Z)-icosa-5,8,11,14,17-pentaenoic) Molecular formula (C20H30O2), CAS (1553-41-9)].

The eicosapentaenoic acid (EPA or also icosapentaenoic is an omega-3(ω-3) polyunsaturated fatty acid (PUFa).

It is a substance that acts by inhibiting the cyclo-oxigenase enzyme. It has been found to help to control weight loss in patients with pancreatic cancer, stabilize protein reserves and fat tissue. This result is followed by the temporary decrease, during the acute phase, in protein synthesis, and energy loss control.

This fatty acid is reported in the state of the art as being able to mitigate the action of catabolic factors in cancer-induced cachexia. Its administration leads to the statistically significant reduction in protein degradation. These results have recommended the eicosapentaenoic acid in the dual role of anti-cancer and anti-cachectic agent in clinical studies. For an example of state of the art research, please see: Beck A S, Smith K L, Tildale M J. Anticachectic and antitumor effect of eicosapentainoic acid and its effect on protein turnover. Cancer Res. 1991; 51:6089-93.

Docosahexaenoic Acid (DHA): [IUPAC: (acid 4Z, 7Z, 10Z, 13Z, 16Z, 19Z-docosa-4, 7, 10, 13, 16, 19-hexaenoic), Molecular formula: (C22H32O2), CAS (6217-54-5)].

The docosahexaenoic acid (DHA) is also considered an omega-3 fatty acid.

The DHA and its metabolites act on the body because of their association with the arachidonic acid (arachidonic acid[IUPAC: eiscosatetraenoic acid cis-5-cis-8-cis-11-cis-14)].

Both compounds (EPA, DHA) have been associated to decrease in cancer volume, weight loss improvement and decrease in anorexia, due to its anti-inflammatory action. According to some authors, the mechanism of reversion of cachexia by EPA consists in the suppression of inflammatory cytokines such as TNFα, IL-1 and IL-6. Examples of state of the art research include (Body J J. The syndrome of anorexia-cachexia. Curr Opin Oncol. 1999; 11(4): 255-60) and (Tisdale M J. The ‘Cancer Cachectic Factor’. Support Care Cancer. 2003; 11(2): 73-8) and (Van Halteren H K, Bongaerts G P A, Wagener D J T H. Cancer cachexia: what is known about its etiology and what should be the current treatment approach? Anticancer Res. 2003; 23(6): 5111-6).

Clinical study involving dietary supplementation in cancer patients with PUFAs, using capsules of fish oil containing 18% of EPA and 12% DHA for 3 months. The results of this test showed decrease in fatigue, reduction of acute phase proteins, and weight gain. Reduction of acute phase proteins (C reactive protein) was associated to suppression in IL-6 production (Inui A. Cancer anorexia-cachexia syndrome: current issues in research and management. CA Cancer J Clin. 2002; 52:72-91).

Glutamine and Arginine: The amino-acids glutamine (GLN) and arginine (ARG) are used as supplementation in nutritional therapy, as components of several nutritious formulas. In the state of the art, there are reports associating the use of glutamine with greater preservation of the skeletal muscle, because of increased protein synthesis and reduction in muscle proteolysis (59, 64, 107, 108.).

Moreover, glutamine improves the nitrogen balance in critically ill patients (increases the function of immune cells without increasing the production of proinflammatory cytokines.

Arginine (ARG), another amino-acid of high nutritional importance in catabolic states is involved in protein synthesis, biosynthesis of amino acids and their derivatives and higher nitrogen retention.

State of the Art—Anorexia-Cachexiatreatment—Available Drugs

There are some drugs used for the palliative treatment of anorexia-cachexia syndrome in the state of the art, and for purposes of explanation only, megestrol acetate, corticosteroids, dronabinol, melatonin, ibuprofen, eicosaminopentoic acid, hydrazine sulfate and also the human growth hormone (somatropin).

Megestrol acetate [-IUPAC (17-Acetyl-17-hydroxy-6, 10, 13-trimethyl-2, 8, 9, 11, 12, 14, 15, 16-octahidro-1H-cyclopenta [a] phenanthren-3-one) CAS 3562-63-8, Molecular formula (C24H32O4) ATC (G03AC05, G03DB02, L02AB0), PubChem (19090) DrugBank (APRD01092)].

The megestrol acetate is a synthetic progesterone derivative. This drug is often used (orally) to treat advanced cancers, cancers responsive to hormone therapy and in the treatment of patients with malnutrition-cachexia syndrome.

In the state of the art, there is a report of the use of the association of medroxyprogesterone acetate with chemotherapy adjuvant in patients with head and neck cancer. Stimulation of appetite and weight gain, improvement of quality of life indicators and decreased serum levels of Interleukin 2 and Interleukin 6 (Mantovani G. et al. Megestrol acetate in neoplasic anorexia/cachexia: clinical evaluation na comparison with cytokine levels in patients with head and neck carcinoma treated with neoadjuvant chemotherapy. Int. J. Clin. Lab. Res. 1995; 25:135-141).

Corticosteroids (Glucocorticoids)—They have been used as palliative treatment of symptoms associated with cancer. They improve, in the short term, appetite, food intake, performance and quality of life of patients, but do not ensure weight gain.

Prolonged treatment with corticosteroids, however, may lead to weakness, osteoporosis and immunosuppression, which can be harmful to cancer patients, since it increases the risk of infection and the evolution of the underlying disease.

Dronabinol [IUPAC (−)-(6aR, 10aR)-6, 6, 9-trimethyl-3-pentyl-6a, 7, 8,10a-tetrahydro-6H-benzo [c] chromen-1-ol.), CAS 1972-08-3, ATC (A04AD10), PubChem (16078)]. It is a synthetic derivative of Cannabis sativa L. in the oral form of tetrahydrocannabinol (THC). Has been used as antiemetic in chemotherapy treatments.

Many studies associate the use of THC as adjuvant in the treatment of cancer patients, with improvement of mood and stimulation of appetite, as well as weight gain. In 1986, dronabinol was approved by the FDA for the treatment of anorexia in AIDS patients and for treating nausea and vomiting in patients undergoing chemotherapy.

Ibuprofen [IUPAC: Acid (RS)-2-[4-(2-methyl-propyl) phenyl] propanoic) Molecular formula: C13H18O2. CAS (15687-27-1), ATC (M01AE01), PubChem (3672) DrugBank (APRD00372)].

Ibuprofen is a non-steroidal anti-inflammatory drug (NSAIDs). It inhibits cyclo-oxygenases and the consequent formation of proinflammatory mediators.

One reported effect of Ibuprofen is the reduction of energy expenditure in patients with pancreatic cancer, suggesting its possible role in the stabilization of the cachectic process, which contributes to weight loss in cancer patients, according to Tisdale M J. Biology of Cachexia. JNCI 1997; 89:1763-73, cited as an example of state of the art research.

Hydrazine sulfate—IUPAC (Hydrazine) Molecular formula (H6N2O4S). CAS (10034-93-2) PubChem (24842).

It is an inhibitor of the phosphoenolpyruvate carboxylase enzyme. Reports in the state of the art demonstrate its beneficial influence in patients with neoplastic cachexia, with weight maintenance or even gain. (Tisdale M J. Biology of Cachexia. JNCI 1997; 89:1763-73.

For other examples of the state of the art, please see: Chlebowski R T, Bulcavage L, Grosvenor M, et al.: Hydrazine sulfate in cancer patients with weight loss. A placebo-controlled clinical experience. Cancer 59 (3): 406-10, 1987,

Melatonin [IUPAC: (N-[2-(5-methoxy-1H-indolo-3-ilo) etilo] ethanamide) Molecular formula: C13H16N2O2 CAS (73-31-4), ATC (N05CM17), PubChem (896), DrugBank (APRD00742)].

Melatonin is a hormone produced in more complex animals by the pineal gland, which can reduce the circulating levels of the Tumor Necrosis Factor alpha (TNF-alfa) in advanced cancer patients. Its clinical use has been associated to reduction of myelosuppression, neuropathy and cachexia in patients with poor clinical conditions with lung cancer.

Somatropin (Recombinant somatropin) [IUPAC: (Human growth hormone), Molecular formula: C990H1532N262O300S7, CAS (12629-01-5), DrugBank (DB00052]. This human growth hormone has anabolic properties and was approved by the FDA for the treatment of the anorexia-cachexia syndrome in patients with AIDS and other chronic diseases, aimed at weight and muscle mass gain.

Other Clinical Complications Associated to Cancer and Also to Treatments—Febrile Neutropenia Associated to Chemotherapy and Other Treatments—State of the Art

Neutrophils are cells in the blood that constitute the first line of defense of the body, participating in the body's defense system, in the presence of infections, particularly bacterial, and tissue lesions. These cells contain granules with proteolytic enzymes that participate in the digestion of antigens.

The so-called neutropenia is clinically characterized by reduction in the number of neutrophils in peripheral blood below their normal values.

Neutropenia is usually defined, in adults and children over one year as the decrease in the number of circulating neutrophils to an absolute cell count below 1.500/mm3 in white individuals and 1.000/mm3 in black individuals. Neutropenia are classified into discrete (1.000 to 1.500 cells/mm3), moderate (500 to 1.000 cells/mm3) and severe (lower than 500 cells/mm3).

Exposure to drugs is one of the most common factors that cause neutropenia. Several drugs may cause neutropenia such as painkillers, antibiotics, anti-inflammatory, antidepressant, anti-thyroid, cardiovascular and cytotoxic drugs, among others.

Neutropenia associated to drugs and other treatments are events particularly damaging to patients with s cancer, because they pave the way for infections caused by opportunistic pathogens.

Additionally, in order to fight opportunistic infections, the use of various methods of treatment and control of these infectious agents is required, particularly antibiotics in high doses, which besides being expensive, may not provide an effective alternative.

Due to the reduction in the number of neutrophils or the limiting in their action, many patients undergoing cancer treatment present clinical pictures of febrile neutropenia, with or without the presence of an identifiable infectious focus.

Febrile neutropenia is a serious complication often induced by the use of cytotoxic agents commonly used in the treatment of cancer and other conditions. Febrile neutropenia is defined as a fever of 38° C. or more maintained for over an hour associated to an absolute count of cells lower than 500/mm3.

Its occurrence associated to cancer chemotherapy may have a negative impact on treatment adherence and on the quality of life of patients.

According to recommendations of the American Society of Clinical Oncology, the incidence of severe neutropenia (neutrophil count below 500/mm3) varies according to the chemotherapy regimen adopted. For an example in state of the art research, see: Smitth T J et al, Update of recommendations for the use of white blood cell growth factors: an evidence-based clinical practice guideline. J Clin Oncol. 2006; 24(19): 3187-205. Epub 2006 May 8.

Treatment of Neutropenia Caused by Cytotoxic Treatments—State of the Art

Neutropenia associated or caused by compounds and/or cytotoxic treatments is considered one of the main problems in the treatment of cancer patients due to their potential for triggering various medical complications, ranging from deterioration in the quality of life of patients to episodes of septicemia and death.

Many compounds used in cancer treatment is associated to significant cytotoxic effects on neutrophils, peripheral blood cell elements that represent the first line of defense of immune response, and, thus, patients receiving cytotoxic chemotherapy have often a decreased immune function and are at higher risk of infection. For an example of state of the art see: Crawford J, Dale D C, Lyman G H. Chemotherapy-induced neutropenia: risks, consequences, and new directions for its management. Cancer 2004; 100:228-237.

Although there are cases of patients with neutropenia associated to the administration of chemotherapy that remain asymptomatic, many patients may experience complications resulting from immunosuppression caused by cytotoxic agents commonly used in chemotherapy, such as fever and infection that may require hospitalization.

The most commonly used treatment is intravenous antibiotic therapy to reduce the risk of opportunistic infections that may lead to septicemia. For an example of state of the art: Over H. New directions in the management of chemotherapy-induced neutropenia: risk models, special populations, and quality of life. Semen Oncol 2003; 30(supple 13):18-23. [145087) One relatively recent therapy in the state of the art to treat neutropenia associated to treatments with cytotoxic drugs is the use of the so-called growth factors of the myeloid lineage (G-CSF and GM-CSF), which are cytokines that regulate proliferation, differentiation and functional activation of hematopoietic cells in the bone marrow. For an example in state of the art: Valley A W. New treatment options for managing chemotherapy-induced neutropenia. Am J Health Syst Pharm 2002; 59: S11-S6.

They are also the cytokines most commonly used for reconstitution of the myeloid series, jointly or sequentially to chemotherapy and radiotherapy treatments, being incontestable in the state of the art its importance for this therapeutic purpose.

They are also extensively used in the state of the art in surgeries that involve the implantation of stem cells and bone marrow cells, for reconstitution of the myeloid series.

Besides their use as stimulants of the myeloid series, experimental data suggest they are also effective when used as adjuvant therapy in patients with some type of cancers, such as invasive and metastatic bladder cancer, contributing for significant increase in survival time. For an example of state of the art Sternberg C N, Mulder P. de, Schornagel J H et al. Seven year update of an EORTC phase III trial of high-dose intensity M-VAC chemotherapy and G-CSF versus classic M-VAC in advanced urothelial tract tumours—European Journal of Cancer 2006:50-54.

They are produced using cell culture technique and other methods. For an example of state of the art, see: U.S. Pat. No. 6,020,169—Production of secreted foreign polypeptides in plant cell culture.

Growth Factors of the Myeloid Lineage—Main Products Available in the State of the Art

Filgrastim [IUPAC (Human granulocyte macrophage colony stimulating factor). Molecular formula (C845H1343N223O243S9)CAS (143011-72-7).

ATC (L03AA02) PubChem (not available) DrugBank (BTD00072)].

Sargramostim [IUPAC (Human granulocyte macrophage colony stimulating factor) Molecular formula (C639H1006N168O19658) CAS (83869-56-1) ATC (L03AA09) PubChem (not available) DrugBank (BTD00035)].

The main adverse side effects related to their use reported in the state of the art are bone pain, varying from mild to intense, and also cases of abnormal proliferation of leukocyte cells, which can lead to discontinuation of treatment.

Problems Associated to Chronic Systemic Diseases and their Treatment—Need for New Therapies and Improvements—General Description of the Present Invention

In view of the aforesaid, it is evident for any expert with knowledge of the state of the art, that new compounds and/or new combinations of drugs or therapies to treat cancer, and also the clinical complications associated to the primary disease (cancer), such as cachexia, and finally the adverse side effects related to the current drugs and treatments should be produced and made available on an ongoing basis.

This is the case of the present invention, that is, it is intended for the treatment of cancer, enhancing synergistically the therapeutic effectiveness of several classes of drugs and other treatments used in combination, and additionally, in an innovative way, can be used concomitantly for treating important clinical problems associated to the primary disease and/or caused or aggravated by the use of other drugs or treatments. The main issues involved in the treatment of cancer, are listed and discussed below, and detailed explanation of the state of the art related to the present invention, as well as indications of the innovative activity involved are provided.

Any animal species can benefit from the present invention. Although the main purpose or intended field of application of the present invention is the treatment of human beings, the invention and its practical use cannot be limited to this species.

No further knowledge or technical expertise is required to the full understanding and use of the present invention.

From the State of the Art to the Present Invention

In the state of the art, the immunomodulator or biological response modifier or immunomodulator called proteic aggregate of ammonium and magnesium phospholinoleate-palmitoleate anhydride, when used alone, is known to have significant antitumor effects in several animal models in cancer research, such as the Walker 256 carcinoma, Ehrlich ascites tumor, breast carcinoma and plasmacytoma. (PI 0305373-3, Ser. No. 10/978,683 and EPA 0426250.3.2405), though not being able, when used alone, to obtain regression of tumor in all cases, as it occurs with the other drugs and treatments described in the state of the art.

In the state of the art, the referred compound (proteic aggregate of ammonium and magnesium phospholinoleate-palmitoleate anhydride) is also known to produce effects on the immune system in experimental animals with various types of cancer, with the production of effector cytokines typical of a TH-1 (IL-2, IFN-gamma) type immune response in PI-0305373-3 Ser. No. 10/978,683 and EPA 0426250.3.2405, with the use of a compound named proteic aggregate of ammonium and magnesium phospholinoleate palmitoleate anhydride alone.

In PI-0305373-3 Ser. No. 10/978,683 and EPA 0426250.3.2405 lymphocyte proliferation and increased NK cell activity are described in animals with tumors as a result of induction of a TH-1 type immune response, with the use of the biological response modifier (proteic aggregate of ammonium and magnesium phospholinoleate-palmitoleate anhydride) alone.

Finally, in the state of the art, the referred compound (proteic aggregate of ammonium and magnesium phospholinoleate-palmitoleate anhydride) is known for its ability not only to induce a TH-1 type immune response with stimulation of endogenous production of effector cytokines typical of such an immune response, as the interferon-gamma (IFN-gamma) and the interleukin-2 (IL-2), but also, when used in combination with other drugs, that is, as an element of a combination antimicrobial therapy, or else as a component in the manufacture of new drugs or drug combinations, it maximizes the antimicrobial action of the referred drug association or combination, creating a synergistic therapeutic action that is wider than the biological properties of the isolated components, without additional toxicity, when it is used to treat infections caused by opportunistic pathogenic microorganisms (WO/2009/097670, U.S. Pat. No. 8,889,153 B2).

The ability to stimulate or cause a TH-1 type immune response with the production of the referred effector cytokines (IFN-gamma, IL-2) is considered in the state of the art in medicine important to fight invasive microorganisms. The referred immune response (TH-1 type immune response) that can be natural or stimulated by some agents or compounds, such as biological response modifiers, is also considered particularly useful in the elimination and/or control of tumors and neoplastic processes.

In the state of the art, there are several reports of experiments in animals, and of therapeutic strategies with cytokines such as IL-2 and Interferons, obtained from exogenous sources, which are used in human beings, in routine clinical practice, aimed to cause or induce a TH-1 type immune response to fight tumors or metastases in cancer patients.

However, in the state of the art, it is known that the use of exogenous cytokines such as Interferons and 1-2 and IL-12, despite their significant therapeutic potential for use alone or as adjuvant immunotherapy drugs when associated to other compounds or combined to other compounds and/or treatments to maximize antineoplastic effects, in order to treat cancer patients, has practical disadvantages, particularly because of their high toxicity levels. For an example in the state of the art, please see: Wiltrout R H et al. Immunotherapy of Cancer by IL-12-based Cytokine Combinations. Expert Opin Biol Ther. 2007 November; 7(11): 1705-1721.

In order to overcome or minimize the problems that commonly occur in the use of adjuvant immunotherapy, that is, when exogenous cytokines are used to treat severe chronic diseases, such as cancer, without compromising the efficiency or usefulness of these substances in the treatment of cancer patients, an innovative and inventive solution can be created and made available through the use of products and compounds that are also able to mobilize and/or stimulate the endogenous or physiological production of such compounds in the body, such as the interferons and the interleukins, preferably without additional toxicity, maintaining these properties in the presence of the cancer process.

This compound is available in the state of the art and is called proteic aggregate of ammonium and magnesium phospholinoleate-palmitoleate anhydride described in PI-0305373-3, WO/2009/097670, U.S. Pat. No. 8,889,153 B2, U.S. Ser. No. 13/516,628) classified as a biological response modifier or immunomodulator because of its ability to act in the immune system.

Thus, it has been specially selected as a useful component of the present invention, which shall play a role similar to that of exogenous cytokines, such as the several types of interferons (IFN-alpha, IFN-beta, IFN-gamma), interleukin-2 (IL-2) and interleukin-12 (IL-12) used in the state of the art as anticancer drugs and/or immunotherapy adjuvants, that is, as effector substances required to trigger an immune response in the patient, in order to enable, restore or enhance the patient's immune system, so that it can efficiently fight or control chronic systemic diseases, such as cancer.

Exogenous cytokines, such as the interferons and interleukins (IL-12, IL-2), among others, are routinely used alone, or, as it is more common in the state of the art, in association or combination with other chemotherapy drugs to maximize the therapeutic effect in the treatment of chronic systemic diseases, such as cancer, though its use alone or in combination with other drugs has some practical disadvantages because of their high toxicity levels. This type of therapeutic strategy is called adjuvant immunotherapy.

The present invention also uses a therapeutic strategy that can be generally described, according to the state of the art in medicine, as a kind of adjuvant immunotherapy. However, in a different and entirely innovative way, the present invention, instead of using exogenous or external cytokines, shall use the patient's body ability to induce an immune response to fight the disease, by stimulating endogenous production of TH-1 effector cytokines that trigger an immune response.

This shall be made possible by the deliberate use of a biological response modifier (proteic aggregate of ammonium and magnesium phospholinoleate-palmitoleate anhydride) specially selected for use in the present invention as the fixed component of a new association or combination of drugs and/or treatments, providing remarkable therapeutic and economic advantages for this new combination or association, as will be detailed in the present report, such benefits being similar to those that would be possible with the use of exogenous cytokines, without facing the problems posed by the use of these exogenous substances.

Therefore, based on this body of knowledge, in the present invention a component specially selected for use in this invention will be associated and/or combined to other drugs and/or treatments, because this compound named in the state of the art as a proteic aggregate of ammonium and magnesium phospholinoleate-palmitoleate anhydride has, among other properties of great interest to the present invention, the ability to produce or stimulate the endogenous production of such effector cytokines that trigger a TH-1 type immune response, considered fundamental in the state of the art for cancer treatment.

Additionally, according to the purpose of the present invention, a new association or combination of drugs and/or treatments of which is part the biological response modifier (proteic aggregate of ammonium and magnesium phospholinoleate-palmitoleate anhydride), will not only provide a new therapeutic association or combination with synergistic therapeutic properties that are distinct from and wider than the properties of the isolated components, but will also allow correction of additional clinical problems associated to the primary disease (cancer) and problems caused by the use of other components of the therapeutic association or combination, which is substantially different from any previous reference to the biological response modifier (proteic aggregate of ammonium and magnesium phospholinoleate-palmitoleate anhydride) or any association or combination described in the state of the art, as shall be detailed in this report, with examples of practical use.

Some Benefits of the Use of the Claimed Invention

Since the therapeutic effectiveness of the association or combination of drugs and/or treatments that characterize the present invention can synergistically be maximized or increased, with the presence in the association or combination of the specially selected biological response modifier (proteic aggregate of ammonium and magnesium phospholinoleate-palmitoleate anhydride), one important consequence is that, depending on the occasion or patient's condition, lower doses or longer intervals can be considered for the use of other drugs and/or treatments selected for the association or combination, particularly when the therapeutic options to be use consist in drugs and/or treatments known to cause cytotoxic or debilitating effects that impact the patient's general condition of quality of life.

Since the toxicity of any drug or treatment that can harm the body as a whole or its components is directly related to the magnitude of the doses used and the duration of treatment, the minimization of adverse side effects and/or its intensity or frequency can be obtained either by the minimization of the required quantities of such other drugs and treatments or by providing a longer period of time for their use, which is made possible by the present invention, as shall be demonstrated in this report, with examples of practical use.

Another benefit provided by the present invention is that it allows the use of higher doses or more intensive regimens than those allowed with other cytotoxic drugs or treatments to be selected for use in association or combination, if needed. This shall occur because of the remarkable ability of the biological response modifier selected as a key component of the present invention (proteic aggregate of ammonium and magnesium phospholinoleate-palmitoleate anhydride) to reverse and/or minimize some of the most harmful effects of such compounds and/or treatments, when present in the combination or association of drugs and treatments described in the invention, as shall be exposed in this report, with examples of practical use.

Many times the need to reduce doses or periods of drug treatments and/or treatments due to the occurrence of side effects directly or indirectly related, mainly to myelosuppression, as shall be detailed in the present report, constitutes a factor that may contribute to the failure of treatments and the invention, and the maximization of the effect of all components of the association or combination made possible by the properties of this invention, shall provide any expert with knowledge of the state of the art with the advantageous option of reducing the doses of drugs, according to the case and the clinical situation involved.

Inversely, in cancer treatment higher doses or more intensive regimens are often necessary to maximize the effect of the drugs or treatments. In both situations, the use of the invention with its unique and surprisingly features may be feasible and useful, representing a considerable improvement in the state of the art compared to the existing therapeutic options or modalities for treatment of cancer.

The toxicity of the biological response modifier specifically selected as a key component of the present invention (proteic aggregate of ammonium and magnesium phospholinoleate-palmitoleate anhydride) is negligible, even at higher doses and does not interfere negatively with the other components, either drugs and/or treatments used in the combination or association, as reported by preclinical and clinical studies, and also according to supplementary data that shall be provided in this report, that is, its use as a key component of the present invention shall not cause any additional toxicity or adverse side effects.

The cited compound (proteic aggregate of ammonium and magnesium phospholinoleate-palmitoleate anhydride), which is a key component of the present invention has already been tested in toxicological studies in the state of the art, with the use of several animal species, including rodents, dogs, nonhuman primates, as well as in clinical trials with human patients, and has not shown any significant toxicity that might discourage its use. For a recent example in the state of the art, please see: Duran N, Nunes I. et al, A biotechnological product and its potential as a new immunomodulator for treatment of animal phlebovirus infection: Punta Toro virus—Antiviral Research, Volume 83, Issue 2, August 2009, Pages 143-147.

Innovative and Integrated Solution for the Main Problems in the Treatment of Cancer

The present invention represents or provides, thus, an association or combination of drugs and/or treatments that expresses or results in practice in a set of novel medicinal effects in the state of the art, for the treatment of cancer, other clinical complications associated and also deficiencies or unwanted side effects of the treatments of these diseases, as shall be detailed in this report, with examples of practical use.

Several aspects related to the treatment of chronic systemic diseases, such as cancer, are described below, which are important for the understanding of the present invention and the level of inventive activity involved, which can be understood by any expert with knowledge of the state of the art, without the need for further explanations:

(A)—The main existing treatments for cancer, are not fully effective to ensure preventive or curative healing.

(B)—In order to maximize the chances of success of the treatment of chronic diseases, such as cancer, chemotherapy treatments or protocols involving several drugs, and also treatment protocols involving, for example, the use of drugs and non-drug procedures, such as surgery and radiotherapy in combination or association, are widely used in the state of the art.

(C)—Chronic systemic diseases, such as cancer, are often preceded and/or followed by malfunctioning or compromised immune system and/or of its important components and/or functions.

(D)—The treatments of these diseases, such as chemotherapy and radiotherapy often suppress the immune system and/or negatively impact the functionality of its components, causing e.g. reduction or destruction of bone marrow stem cells, which is expressed in the amount and functionality of peripheral white blood cells, such as lymphocytes and neutrophils.

(E)—Adverse clinical conditions associated to the primary disease, such as cachexia or anorexia-cachexia syndrome, characterized by weight loss and accelerated depletion of muscle and fat tissue by most sufferers of chronic systemic diseases, such as cancer, are common findings in cancer patients and be aggravated by existing treatments against cancer in the state of the art.

Ideal Characteristics of a New Treatment: The Invention Possesses all Desirable Characteristics for the Treatment of Cancer and Related Comorbidities

Considering all these aspects, it would be highly desirable and innovative that a treatment or drug for chronic systemic diseases, such as cancer, meets all these needs, as follows:

1) Increase the effectiveness or success rate of the existing drugs and/or treatments,

2) Recover or increase the effectiveness of the immune system and/or functionality of its components impaired by the disease,

3) Counteract or reduce the incidence or severity of side effects on cells of the immune system caused by the use of other drugs or treatments,

4) Act in a preventive, curative or palliative way regarding the important adverse clinical condition associated to these diseases and/or aggravated by the other drugs and/or treatments called cachexia,

5) Substantially improve the quality of life of patients.

The present invention consists precisely in a new combination of drugs and/or treatments which in a remarkable and innovative way, satisfies all the requirements of the aforementioned five ideal features of a new treatment, that is, those listed in items 1, 2, 3, 4 and 5.

Satisfaction of all these requirements or features was obtained in the invention, as it shall be demonstrated in the present report, with the use of the biological properties of the immunomodulator (proteic aggregate of ammonium and magnesium phospholinoleate-palmitoleate anhydride), which were combined in an innovative way with the therapeutic properties of other existing drugs and/or treatments, and, in a remarkable way, can maximize synergistically the therapeutic effect of the association or combination against cancer, eliminate or minimize suppression of the immune system often associated to the existing treatments, reverse or minimize primary and secondary cachexia and consequently improve the quality of life of patients.

For the purposes of the present invention, the biological and therapeutic properties of the biological response modifier (proteic aggregate of ammonium and magnesium phospholinoleate-palmitoleate anhydride), are added to the biological and therapeutic properties of the other drugs and/or treatments, when used in association or combination, creating a synergistic action wider than the biological properties of the isolated components.

Concomitantly and surprisingly, as shall be detailed in the present report, the use of the biological response modifier (proteic aggregate of ammonium and magnesium phospholinoleate-palmitoleate anhydride), specially selected for the present invention shall provide a remarkable contribution to reverse or minimize other symptoms and clinical pictures associated to the disease, such as cachexia, and in an innovative way will make it possible to reverse or minimize unwanted side effects on the immune system or its components used in association or combination, aimed to improve the general condition of the patient, positively impacting their quality of life.

That is, the outcome or the practical effect of the invention, or else, of the association of combination of several classes of medications and modalities of treatments available in the state of the art with the use of the biological response modifier (proteic aggregate of ammonium and magnesium phospholinoleate-palmitoleate anhydride), specifically selected for use as a key component and always present in any association or combination with these other elements, not only will have wider therapeutic effectiveness than any of its components individually considered, but will also make it possible to correct or minimize important adverse side effects of the other components that create obstacles to their use alone, as shall be shown in this report.

The present invention, a new association or combination of drugs and/or treatments that uses specifically the biological response modifier called in the state of the art proteic aggregate of ammonium and magnesium phospholinoleate-palmitoleate anhydride with other drugs and/or treatments for chronic systemic diseases, such as cancer, with the deliberate purpose of maximizing the therapeutic effectiveness of the association and also reverse or minimize immunosuppression or loss of functionality of the immune system and/or of any of its components, and finally treat, in an effective way, another clinical problem typical of this type of disorder (cachexia), either it is caused by the base disease and/or aggravated by other treatments, with strong positive impact on the quality of life of users, has not been described in the state of the art until now with these unique and remarkable characteristics and properties.

Although the invention uses components of several classes and types of drugs and/or treatments already known in the state of the art, it allows to obtain new effects for these drugs and/or treatments, when associated or combined, that have not been described in the state of the art until now, that is, possessing much more powerful therapeutic properties completely different than one might expect to obtain with the isolated use of its components, and which can be understood by any expert with knowledge of the state of the art, particularly after the explanations contained in the present report and the practical examples to be provided.

For explanatory purposes, it can be said that the role of the biological response modifier called in the state of the art proteic aggregate of ammonium and magnesium phospholinoleate-palmitoleate anhydride in the association or combination of drugs or treatments that characterizes the present invention can be understood as a type of adjuvant immunotherapy. The referred compound has a role similar to that of interferons and other exogenous cytokines, in the drug combinations or associations already known in the state of the art and mentioned in this report.

However, because of the evident benefits arising from the use of the biological response modifier (proteic aggregate of ammonium and magnesium phospholinoleate-palmitoleate anhydride) of being able to induce endogenous formation of interferon-gamma (IFN-gamma) and other key substances, such as cytokines (IL-2) that are essential to the proper functioning of the immune system in the face of disease, and as shall be seen in this report, the presence in the association or combination of this biological response modifier (proteic aggregate of ammonium and magnesium phospholinoleate-palmitoleate anhydride) also allows to add other remarkable and innovative therapeutic properties to the invention, with a much wider action than those reported in the state of the art, also for the biological response modifier (proteic aggregate of ammonium and magnesium phospholinoleate-palmitoleate anhydride) and for these other cytokines either isolated or in association, such as the ability to provide means of treatment for other clinical complications arising from the primary disease and, finally, the ability to solve or minimize adverse side effects of other drugs and treatments.

In view of these differentiating characteristics, the invention not only has wider action or therapeutic power than other associations or combinations of drugs and/or treatments in the state of the art that use the so-called adjuvant immunotherapy with the aforementioned cytokines, though from exogenous sources, because of its ability, conferred by the biological response modifier, of inducing or stimulating endogenous production of effector cytokines that trigger immune response, such as interferons (IFN-gamma) and interleukins (IL-2), and, if necessary and depending on the specific type of treatment for the patient, may allow the physician to consider minimizing the use of exogenous cytokines, such as interferon, as well as other exogenous cytokines and/or reduce their doses when the regimens prescribed involve their association with other drugs, among other benefits.

This ability or property of the present invention, that is, physiological or endogenous induction of effector cytokines that trigger TH-1 type immune response, such as the IFN-gamma and IL-2, resulting from the use of the biological response modifier (proteic aggregate of ammonium and magnesium phospholinoleate-palmitoleate anhydride) in the invention also allows to obtain a significant economic advantage, besides the therapeutic benefits: Minimization or elimination of the occurrence of intolerance reactions or toxicity in patients, which are common side effects of the use of these cytokines that modulate immune response, such as interferons and interleukins obtained from exogenous sources, which often demand additional costs for their control.

For an expert with knowledge in the state of the art, the benefits of using substances of physiological or endogenous origin to replace or minimize the necessary amounts of the same substances, though obtained from exogenous sources, in the treatment of diseases, by minimizing the risk of adverse side effects such as allergic reactions are evident and need no further explanation.

The invention can be used in two ways:

1) Use of the biological response modifier (proteic aggregate of ammonium and magnesium phospholinoleate-palmitoleate anhydride) associated to regimens or protocols consisting in the combination of this specific immunomodulator with other drugs and/or non-drug treatments, the latter empirically chosen or selected by medical professionals at the beginning of therapy, among those available in the state of the art and that are deemed more appropriate to be used with the biological response modifier (proteic aggregate of ammonium and magnesium phospholinoleate-palmitoleate anhydride), depending on patient and disease state, as shall be detailed in the present report.

2) Use of the biological response modifier (proteic aggregate of ammonium and magnesium phospholinoleate-palmitoleate anhydride) as a component or part in the manufacture of new drugs, when combined or associated with other drugs of the most different types and categories, as shall be detailed in this report.

An in order to ensure that the present invention is perfectly understood, some practical examples of its use in different tumors and pathologies of interest follow, as well as some comments and examples of use of other substances and associations that have not been described in the state of the art until now as therapies against these disorders.

These examples are also provided to help understanding the many possibilities and benefits provided by the invention for the treatment of chronic systemic diseases such as cancer, associated clinical problems and also for the correction of problems associated to other drugs and/or existing treatments in the state of the art.

It can be affirmed that any expert with knowledge in the state of the art based only on the information and practical examples to be provided in the present report should be able to fully understand and use the present invention to its fullest extension.

These practical examples are merely illustrative and do not intend to limit the scope of the present invention.

### PRACTICAL EXAMPLE

### Use of the Present Invention for the Treatment of Tumors in Experimental Animals—Use of Two Biological Response Modifiers

Several experiments were conducted using the same experimental model of Lewis lung carcinoma (3 LL) in animals, where the therapeutic effect of the biological response modifier (proteic aggregate of ammonium and magnesium phospholinoleate-palmitoleate anhydride) used alone was assessed. Also, for comparative purposes, the present invention, that is, the biological response modifier (proteic aggregate of ammonium and magnesium phospholinoleate-palmitoleate anhydride) associated to another biological response modifier (Il-2) was assessed, as well as the biological response modifier (proteic aggregate of ammonium and magnesium phospholinoleate-palmitoleate anhydride) used in association with cytotoxic compounds (vindesine sulfate and cisplatin).

In the first experiment (Table A) the production of cytokines and survival provided by the biological response modifier (proteic aggregate of ammonium and magnesium phospholinoleate-palmitoleate anhydride) in the animals treated with the biological response modifier (proteic aggregate of ammonium and magnesium phospholinoleate-palmitoleate anhydride) used alone and in combination with another biological response modifier called Interleukin-2 (Il-2) was assessed for comparative purposes. The referred compound (Il-2) is widely use in the treatment of cancer in the state of the art. Data from this first experiment is shown in Table A.

In the second experiment (Table B) the use of the immunomodulator (proteic aggregate of ammonium and magnesium phospholinoleate-palmitoleate anhydride) used alone and in combination with two other cytotoxic compounds: vindesine sulfate and cisplatin, available in the state of the art and widely used in the treatment of cancer, were comparatively assessed in experimental animals. The data of this second experiment are contained in Table B and Table C.

Association or Combination of the Biological Response Modifier (Proteic Aggregate of Ammonium and Magnesium Phospholinoleate-Palmitoleate Anhydride) with Other Biological Response Modifiers and Chemotherapy Drugs

General Experimental Drawing

Animals: 3-week-old C57Bl/6 female mice were used in all the experiments (Table A, Table B, Table C)

Tumor: Lewis lung carcinoma (3LL) was the tumor selected for all the in vivo assessments (Table A, Table B, Table C) in these experiments, with the cells maintained in culture medium and inoculated subcutaneously in the animals at the concentration of 6×105 cancer cells (3 LL) for each animal.

Biological response modifiers used (Table A and Table B):

a) Proteic aggregate of ammonium and magnesium phospholinoleate-palmitoleate anhydride

b) Interleukin 2 (IL-2)

Cytotoxic chemotherapy drugs used (Table B and Table C)

c) Vindesine sulfate

d) Cisplatin

Treatments: All treatments began 24 hours after the inoculation of cancer cells.

Observation period: The animals were evaluated during an observation period of 100 days in the survival experiment.

The animals used in the quantification of cytokines were observed until the 21st day.

Serum cytokine levels: In order to quantify the cytokine level in serum from mice, the blood samples were collected in the mice from retro-orbital puncture at the 21st day after the beginning of treatment and separate plasma pools were made from the blood samples.

The blood samples were maintained at 4° C. for 24 hours for clot retraction and were then centrifuged at 2700 rpm for 30 minutes. Quantitative analyses of IFN-gamma and IL-2 were made with the kit BD TM Cytometric Bead Array—Mouse Th1/Th2 Cytokine CBA (BD Biosciences, CA-EUA).

Experimental Protocol

1st Experiment—Practical effect of the invention—Animals treated with the biological response modifier (Proteic aggregate of ammonium and magnesium phospholinoleate-palmitoleate anhydride) associated to Interleukin 12 (IL-12). (Table A).

Animals: 3-week-old C57Bl/6 female mice

Tumor model: Lewis lung carcinoma (3LL) in the 6×105 concentration of cancer cells (3 LL) for each animal.

Biological response modifiers used: (Table A and Table B):

a) Proteic aggregate of ammonium and magnesium phospholinoleate-palmitoleate anhydride

b) Interleukin 2 (IL-2)

Two batches of 20 mice (C57BL/6) each one, inoculated with 3 LL cells (6×105 cells/animal) were treated only with daily intraperitoneal injections of the biological response modifier (proteic aggregate of ammonium and magnesium phospholinoleate-palmitoleate anhydride), as follows: 5.0 mg/kg/daily dose (Table A-Group 1-IM-1) and 0.5 mg/kg/daily dose (Table A-Group 2-IM 2), applied in three 3 weekly cycles. The animals were followed up for 100 days.

A third batch (Table A-NaCl-Group 3) with 20 animals was also inoculated with 3 LL cells (6×105 cells/animal), treated only with 0.2 ml of saline solution (NaCl 0.9%) and followed up for an equal period of time for control purposes.

A fourth batch of 20 animals (Group 4) inoculated with 3 LL cells (6×105 cells/animal), was treated with 0.5 mg/kg/day of the proteic aggregate of ammonium and magnesium phospholinoleate-palmitoleate anhydride (Table A-Group 4-IM-2) and Interleukin-12 (IL-12), 0.05 ug/animal given jointly by intraperitoneal injections in 3 weekly cycles. The animals were followed up for 100 days.

A fifth batch of 20 animals (Table A-Group 5) inoculated with 3 LL cells (6×105 cells/animal) was treated with Interleukin-12 (IL-12), 0.1 ug/animal applied by intraperitoneal injection in 3 weekly cycles. The animals were followed up for 100 days, and the number of survivors was expressed in percentage.

The results are shown in Table A:

TABLE A
groups, drugs and dosages

| | Days | % Survivors |
|---|---|---|
| Group 1 IM-1 (5 mg/kg) | 100 | 40 |
| Group 2 IM-2 (0.5 mg/kg) | 100 | 20 |
| Group 3 (Nacl 0.9%) | 100 | 3 |
| Group 4 IM-2 (0.5 mg/kg) + | 100 | 96 |
| Il-2 (0.05μ g/animal) | | |
| Group 5 (IL-2-0.1 μg/animal) | 100 | 50 |

2° Experiment—Practical effect of the invention—Animals treated with the biological response modifier (Proteic aggregate of ammonium and magnesium phospholinoleate-palmitoleate anhydride) associated to vindesine sulfate and cisplatin (Table B and Table C).

Animals: 3-week-old C57Bl/6 female mice Tumor model: Lewis lung carcinoma (3LL) in the 6×105 of cancer cells (3 LL) for each animal.

Cytotoxic chemotherapeutic drugs used (Table B and Table C)

c) Vindesine sulfate

d) Cisplatin

Cytokine measurement (IFN-gamma and IL-2): kit BD TM Cytometric Bead Array—Mouse Th1/Th2 Cytokine CBA (BD Biosciences, CA-EUA)—(Table C).

Two batches of 20 mice (C57BL/6), with 10 males and 10 females each, transplanted with cells of Lewis lung carcinoma (3LL-6×105 cells/animal) were treated only with the proteic aggregate of ammonium and magnesium phospholinoleate-palmitoleate anhydride, intraperitoneally given at the 5.0 mg/kg/daily dose (Table B-group 1) and 0.5 mg/kg/daily dose (Table B-group 2), applied in 3 weekly cycles that started 24 hours after tumor inoculation. The animals were followed up for 100 days (Table B).

A third batch (Table B-group 3) with 20 animals was equally inoculated with 3 LL cells (6×105 cells/animal), treated only with 0.2 ml of saline solution (NaCl) at 0.9%, intraperitoneally given 3 times a week. The treatment began 24 hours after tumor inoculation and the animals were followed up for 100 days (Table B).

A fourth batch of 20 animals (Table B-group 4) inoculated with 3 LL cells (6×105 cells/animal), was treated with 0.5 mg/kg/day of proteic aggregate of ammonium and magnesium phospholinoleate-palmitoleate anhydride and vindesine sulfate (3 mg/kg) and cisplatin (1 mg/kg) jointly given by intraperitoneal injection 24 hours after tumor cell inoculation, in 3 weekly cycles. The animals were followed up for 100 days (group 4-Table B).

A fifth batch of 20 animals (Table B-group 5) inoculated with 3 LL cells (6×105 cells/animal) was treated only with vindesine sulfate (3 mg/kg) and cisplatin (1 mg/kg) administered together by intraperitoneal injection 24 hours after tumor cell inoculation, in 3 weekly cycles. The animals were followed up for 100 days (Table B).

A sixth batch of animals (Table C-Group 6) was treated only with saline solution (NacL 0.9%) intraperitoneally given 24 hours after tumor cell inoculation in 3 weekly cycles. The animals under this experiment were sacrificed on the 21th day after the beginning of the treatment for the measurement of the IL-2 and Interferon-gamma levels (Table C-Group 6).

A seventh batch of 20 animals (Table C-Group 7) inoculated with 3 LL cells (6×105 cells/animal) was treated with 0.5 mg/kg/day of proteic aggregate of ammonium and magnesium phospholinoleate-palmitoleate anhydride and vindesine sulfate (3 mg/kg) and cisplatin (1 mg/kg) administered together by intraperitoneal injection 24 hours after tumor cell inoculation, in 3 weekly cycles. These animals were also sacrificed on the 21° day after the application for the measurement of the IL-2 and Interferon-gamma levels (Table C-Group 7).

An eighth batch of 20 animals (Table C-Group 8) inoculated with 3 LL cells (6×105 cells/animal, was treated only with vindesine sulfate (3 mg/kg) and cisplatin (1 mg/kg) administered together by intraperitoneal injection 24 hours after tumor cell inoculation in 3 weekly cycles. These animals were sacrificed on the 21° day after the beginning of the treatment for the measurement of the IL-2 and Interferon-gamma levels (Table C-Group 8).

The cytokines (IFN-gamma and IL-2) were measured with the use of a BD TM Cytometric Bead Array—Mouse Th1/Th2 Cytokine CBA (BD Biosciences, CA-EUA) kit.

The results of these experiments are shown in Table B, for the percentage of survivors, and in Table C, for the cytokine levels (IFN-g and IL-2).

TABLE B
Percentage of survivors

| | 21 Days (%) survivors | 100 Days (%) survivors |
|---|---|---|
| Group 1 | 100 | 41 |
| Group 2 | 100 | 19 |
| Group 3 | 100 | 18 |
| Group 4 | 100 | 90 |
| Group 5 | 100 | 60 |
| Group 6 | 0 | — |
| Group 7 | 0 | — |
| Group 8 | 0 | — |

TABLE C

| | Days after the | | |
|---|---|---|---|
| Levels of IFN-g/ | beginning of | Interferon-gamma | Interleukin-2 |
| IL-2 | treatment | (IFN-g) pg/ml | (IL-2) pg/ml |
| Group 6 (control) | 21 | 1256 ± 106 | 804 ± 75 |
| Group 7 | 21 | 1448 ± 80 | 890 ± 60 |
| Group 8 | 21 | 857 ± 38 | 730 ± 35 |

Discussion of the results—Practical use of the invention—Association of the biological response modifier (proteic aggregate of ammonium and magnesium phospholinoleate-palmitoleate anhydride,) and Interleukin-12.

The use of the present invention, that is, the association or combination of the biological response modifier (proteic aggregate of ammonium and magnesium phospholinoleate-palmitoleate anhydride) in the practical experiment contained in Table A has clearly demonstrated the use of the invention, consisting, in this case, in an association of the biological response modifier (proteic aggregate of ammonium and magnesium phospholinoleate-palmitoleate anhydride) with another biological response modifier, in this case, Interleukin-12 (IL-12) has a much more efficient therapeutic action regarding the survival of test animals assessed 100 days after the beginning of the experiment.

A it can be seen in Table A, the mice group given only the biological response modifier (proteic aggregate of ammonium and magnesium phospholinoleate-palmitoleate anhydride) alone had a 20% survival percentage (table A-Group-2-IM-2-0.5 mg/kg) and 40% (Table A-Group 1-IM-1-5.0 mg/kg), respectively, 100 days after tumor cell inoculation, and the group of animals treated only with IL-12 (group 5-IL-12-0.1 IFN-g/animal) had a survival rate of 50%.

Although the survival rate of animals treated with the biological response modifier (proteic aggregate of ammonium and magnesium phospholinoleate-palmitoleate anhydride) alone at its highest dosage (group Table A-1-IM-1-5 mg/kg), that is, 40% of the survivors is very similar to the survival rate of the group treated only with 11-12 (Table A-Group 5-IL-2-0.1 ug/animal) with a survival rate of 50%, the experiment reveals the remarkable effectiveness of the use of the present invention the use of the invention regarding the percentage of surviving animals.

It can be clearly seen that the therapeutic effectiveness of the invention, that is, of the combination of the biological response modifier (proteic aggregate of ammonium and magnesium phospholinoleate-palmitoleate anhydride) and Interleukin-12 (IL-12) is clearly greater than the effect obtained with the use of any of the other two compounds when used alone, regarding the percentage of surviving animals at the end of the experiment, which is a 96% percentage of survivors (Table A-Group 4).

Interestingly, the doses of the biological response modifier (proteic aggregate of ammonium and magnesium phospholinoleate-palmitoleate anhydride) and Interleukin 12 (IL-12) needed or used, when administered in the combination form made available for usage by the present invention, and with the purpose of obtaining a much greater therapeutic effect (Table A-Group 4) are much lower than those previously used, when administered alone, since the dose used in the association or combination for IL-12 to obtain a 96% survival rate (Table A-Group 4) is half the dose previously used, that is, 0.05 ug/animal (table A-Group 4) and the dose of the biological response modifier (proteic aggregate of ammonium and magnesium phospholinoleate-palmitoleate anhydride) is 0.5 mg/kg (Table A-Group 4).

It can be clearly seen that the use of the invention in this practical example, that is, the combination of the biological response modifier (proteic aggregate of ammonium and magnesium phospholinoleate-palmitoleate anhydride) with an exogenous cytokine, that is, the Interleukin-12 (IL-12) has a therapeutic effectiveness, in what concerns the number of surviving animals (96%-Table A-Group 4), much higher than the best results obtained for any of the other two compounds when used alone, that is, 40% of survivors (group 1-Table A) for the biological response modifier (proteic aggregate of ammonium and magnesium phospholinoleate-palmitoleate anhydride) and 50% for interleukin-12 (group 5-table A).

Therefore, a higher therapeutic effectiveness was obtained with the use of the present invention (Table A-Group 4), which is attested by the rate of surviving animals, with the administration of lower doses of both compounds, biological response modifiers, compared to the findings obtained for any of the other two aforementioned compounds, when used alone in this experiment.

Practical use of the invention—Association of the biological response modifier (proteic aggregate of ammonium and magnesium phospholinoleate-palmitoleate anhydride,) and cytotoxic drugs (Vindesine sulfate and cisplatin)—(Table B and Table C).

When the invention is used in another experiment reproducing a common situation in the state of the art, that is, a protocol of cytotoxic chemotherapy drugs in association (vindesine sulfate and cisplatin) with the necessary use, associated or combined, of the biological response modifier (proteic aggregate of ammonium and magnesium phospholinoleate-palmitoleate anhydride) the results obtained are similar (Table B).

Analysis of data in Table B, that is, the results obtained for vindesine sulfate and cisplatin (Table B) shows that the biological response modifier (proteic aggregate of ammonium and magnesium phospholinoleate-palmitoleate anhydride) used alone resulted in a survival rate of 41% of the experimental animals, when administered at a dose of 5 mg/kg (Group 1-Table B) and a survival rate of 19% for the same compound when used alone at a dose of 0.5 mg/kg (Group 2-Table B), which is almost the same effect obtained in the control group that received only 0.9% of Nacl (control group-group 3-Table B) and that had 18% of surviving animals after 100 days (Group 3-Table B).

The group that was given the combination of drugs/therapies that constitute the present invention, that is, the biological response modifier (proteic aggregate of ammonium and magnesium phospholinoleate-palmitoleate anhydride) at a dose of 0.5 mg/kg, associated to vindesine sulfate at a dose of 3 mg/kg and cisplatin (1 mg/kg) in 3 weekly cycles resulted in a survival rate of 90% in 100 days (Group 4-Table B), which is much higher than the survival rate of 60% (Group 5-Table B) obtained for the same dose of vindesine sulfate (3 mg/kg) and cisplatin (1 mg/kg) in 3 weekly cycles, however without the presence of the specific biological response modifier.

As it can be seen in the data from Table B, the use of the invention, that is, the biological response modifier (proteic aggregate of ammonium and magnesium phospholinoleate-palmitoleate anhydride) used in the lowest dose, that is, at the dose of 0.5 mg/kg, though associated to vindesine sulfate and cisplatin (Group 4-Table B), have made it possible to obtain a survival rate of 90% (Group 4-Table B), compared to the mere 19% when the biological response modifier was administered alone at the same dose of 0.5 mg/kg, (Group 2-Table B).

However, this 90% survival rate (Group 4-Table B) obtained with the use of the invention is also much higher than the result obtained with the combination of the cytotoxic agents only (chemotherapy protocol) used without the biological response modifier (proteic aggregate of ammonium and magnesium phospholinoleate-palmitoleate anhydride), which was only a 60% survival rate (Group 5-Table B).

Regarding the production of cytokines with effector mechanisms of immune response, it can be seen in Table C (cytokine quantification) a significant rise in the IFN-g and IL-2 levels for the group that used the combination of drugs/therapies that constitute the present invention (Group 7-Table C) compared to the levels found for the group that was given only the two cytotoxic compounds associated (Group 8-Table C).

Although the levels of effector cytokines and/or cytokines associated to a TH-1 type immune response) in the group that used the combination of drugs/therapies that constitute the present invention (Group 7-Table C) are similar to the levels found in the control group (Group 6-Table C), a significant rise was observed compared to the group that was given only the association of cytotoxic compounds (Group 8-Table C), which is very relevant, given the neoplastic process and the use of immunosuppressive compounds (cisplatin).

Presumably, the higher survival rates obtained for the group treated with the combination of drugs/therapies that constitute the present invention (Group 4-Table B) can be associated to the stimulation of the production of effector cytokines of the immune response (IFN-g and IL-2) caused by the presence of the biological response modifier (proteic aggregate of ammonium and magnesium phospholinoleate-palmitoleate anhydride) in combination or association with cytotoxic compounds (Group 7-Table C), a hypothesis that is corroborated by the levels of these cytokines compared to the levels in the control group (Group 6-Table C) and particularly the levels of effector cytokines compared to the levels obtained with the use of the association of cytotoxic compounds only (Table C-Group 8).

The practical effect of the invention does not depend on the knowledge or elucidation of possible mechanisms of action of any components, including the increase in cytokine levels caused by the presence of the biological response modifier (proteic aggregate of ammonium and magnesium phospholinoleate-palmitoleate anhydride).

However, the practical result of the experiments shown in Table C is important because it reveals that the use of the invention, that is, the biological response modifier (proteic aggregate of ammonium and magnesium phospholinoleate-palmitoleate anhydride) leads to increased endogenous production of the levels of cytokines with effector mechanisms of immune response (IFN-g and IL-2), and more importantly, that this effect is maintained in the presence or with the use of cytotoxic and immunosuppressive drugs.

The ability of the present invention (Table C), or else, the endogenous induction of cytokines with effector mechanisms of immune response, even in the presence of a chronic systemic disease (cancer) and of immunosuppressive drugs, reveals that besides triggering an immune system response (TH-1 response) associated to the state of the art as necessary to assist the response or reaction of the host body to neoplasias, it shows the full effectiveness of this invention, that is, of the biological response modifier (proteic aggregate of ammonium and magnesium phospholinoleate-palmitoleate anhydride) of causing an (endogenous) increase in the number of cytokines with effector mechanisms of immune response (IFN-g and IL-2), or else, in clinical practice, its use in the invention shall also counterbalance the immunosuppressive effect of other drugs that might be used in association or combination.

The remarkable property of the invention, that is, the endogenous production of effector cytokines (IFN-gamma and IL-2), confirms the effectiveness of the invention in practical situations where experts with knowledge of the state of the art who would no longer need to use exogenous cytokines, or in the reduction of cytokine doses in chemotherapy protocols where such use is associated to other drugs or non-drug treatments.

Finally, it can be easily seen in data from Table B that the present invention, that is, the association or combination of the biological response modifier (proteic aggregate of ammonium and magnesium phospholinoleate-palmitoleate anhydride) is also fully effective for use in treatment regimens that include more than one chemotherapy drug (chemotherapy protocols) with therapeutic gains for the combination or association, or else, generating medical benefits greater than those obtained with the use of any of the isolated compounds (Group 4-Table B).

For the purposes of the present invention, that is, for treatment of cancer, the use of the biological response modifier must or shall be associated to at least one compound, drug or other non-drug treatment. More than one compound, drug or non-drug treatment can be used, including in the form of combination or association between these other elements.

These data of the practical example (Table B and Table C) also reveal one of the useful aspects of this invention, because it not only proves that the invention has synergistic therapeutic action than the therapeutic action of the isolated components of the association or combination. Also, it can be affirmed that the present invention will make it possible to use lower doses of the agents or compounds to obtain a greater effect than that obtained with higher doses of the isolated compounds.

This will create significant therapeutic, economic and quality-of-life benefits that can be clearly understood by any expert with knowledge of the state of the art without the need for further explanations.

New Data Concerning the Use of Invention in the Treatment of Prostate Cancer, Pancreatic Cancer, Ovariann Cancer, Lung Cancer in Animals and Human Subjects

In the parent application (U.S. application Ser. No. 13/516,628) several examples of synergistic effects concerning the use of the invention against cancer and the concomitant treatment of comorbidities that are related with the cancer and the therapies was provided.

To provide more information about the usefulness of the claimed invention to treat cancer and the concomitant treatment of the comorbidities related to the cancer and its treatments, a new series of experiments using new combinations of drugs was performed using animal models for study of prostate cancer, pancreas cancer, lung cancer and ovarian cancer are included in this specification.

Reports concerning cases of patients suffering from prostate cancer, pancreas cancer and small lung cells carcinoma treated with the claimed invention are also included herein.

A refreshment of the data of the previous clinical trials is also provided.

New data to complement the information about the use of the invention in the treatment of premalignant lesion is also available.

The new data and examples are described in the specification below.

Examples of Practical Use of Invention—Animal Model—Prostate Cancer—Immunomodulator Used in Combination with Antiandrogen Therapy and Chemotherapics

### State of Art

Prostate cancer (PCa), after skin cancer, is the second most common cancer among men, but it can often be treated successfully by a combination of drugs and non-drugs therapies when prematurely detected.

Patients with other carcinogenic process in different anatomical sites of the body may also develop prostate cancer.

For instance, carcinoma in situ involving the prostatic urethra is not an uncommon finding in advanced bladder cancer patients (National Comprehensive Cancer Network, 2014). Some studies have pointed that patients with bladder cancer have higher incidence of PCa and vice versa (Kellen et al., 2007).

These findings suggest that these cancers may share a common carcinogenic process or also that these patients are particularly susceptible to both cancers (Kinoshita et al., 2004; Izumi et al., 2014).

Besides the existence of several therapies such as surgery and radiotherapy to eradicate or manage PCa, this disease remains a challenge due the potential of PCa in developing metastases, combined with the low efficacy of the drug therapies available to be used as adjuvants with surgery and radiotherapy.

The low level of effectiveness of current drug therapies against prostate cancer (PCa) may be related to modulation of steroid hormone receptors in the mechanisms of tissue repair and angiogenesis and reactive oxygen species (ROS). In many cancers, the interaction of steroid hormone receptors with the ROS increases primary lesions triggering the rapid progression and increasing the malignancy of lesions.

Human prostate cancer, in the state of art, is treated using preferentially surgery, radiotherapy in combination with some adjuvant drug therapies, such as that is used for androgen deprivation therapy.

The rationale of use of androgen deprivation therapy in the treatment of prostate cancer is that the androgens (e.g. testosterone) stimulate the growth of prostate cancer cells. Therefore lowering androgen levels or stopping them from getting into prostate cancer cells often makes prostate cancers shrink or grow more slowly for a time. For this goal, because androgens have to bind an androgen receptor inside the cells to work, the anti-androgens drugs act binding preferentially to these receptors and avoid that the androgens make the same. Consequently, the androgen levels decrease and the growth of prostate cancer cells is impaired.

As cited above, anti-androgen therapies alone are not fully effective against prostate cancer. Therefore, such therapy may be used as adjuvant therapy with surgery, radiotherapy and other chemotherapies aiming to maximize the effectiveness of treatments.

The multifaceted nature of the process of angiogenesis in malignant tumors suggests that antiangiogenic drugs, used in combination or association with agents that modulate pro and antioxidants species as well those that modulate steroid receptors such as anti-androgens (e.g. Flutamide) may be more effective than therapies involving only a single agent. In this context, the development of immunotherapies against cancer, including prostate cancer (PCa), may represent a valuable treatment option (Schweitzer & Drake, 2014).

Thus, taking into account the remarkable properties of the invention against cancer, one association of the immunomodulator (proteic aggregate of ammonium and magnesium phospholinoleate-palmitoleate anhydride) with a steroid blocking receptor (Flutamide) was tested in animal model. The results are shown below.

Example of Practical Use of Invention—Immunomodulator Plus Anti-Androgen Therapy (Flutamide)—Animal Model for Prostate Cancer

Experimental Design

A number of 100 F344 (Fischer 344) male rats were used in the experiments using the immunomodulator (proteic aggregate of ammonium and magnesium phospholinoleate-palmitoleate anhydride).

The rats were housed individually and had free access to ration and water. At 10 weeks of age the animals were randomly distributed into 5 groups. For induction of prostatic lesions the animals received daily 100 mg/Kg dose of testosterone cypionate subcutaneously (s.c.) for 5 consecutive days.

After this initial procedure, the animals were anesthetized for inoculation of 15 mg/Kg dose of N-methyl-N-nitrosourea (MNU) in the ventral lobe of the prostate capsule in one week, totalizing two doses. One week after the last dose of MNU (Sigma Chemical Co., St Louis, Mo., USA), the animals received supplementary doses of 5 mg/kg/day of testosterone cypionate subcutaneously for more two weeks.

After 120 days of PCa induction, all animals were submitted to ultrasound exams to check for PCa and subsequently subdivided into five groups (20 animals per group):

Cancer control group (Group 1): received 5 ml/kg dose of 0.9% saline solution (s.c.), three times per week for 60 days;

Cancer+immunomodulator group (Group 2): received 5 mg/kg/s.c dose of the immunomodulator (s.c.), three times per week for 60 days;

Cancer+Flutamide (Group 3): received 10 mg/kg dose of Flutamide (s.c.) three times per week for 60 days;

Cancer+Immunomodulator+Flutamide (Group 4): received sequentially treatment with the immunomodulator (2 mg/kg/sc) and Flutamide (5 mg/kg/s.c.) three times per week for 60 days;

Cancer+Immunomodulator+Flutamide (Group 5): received concomitant treatment with the immunomodulator (2 mg/kg/sc) and Flutamide (5 mg/kg/s.c.) three times per week for 60 days;

After 60 days of treatment, 10 animals of each group were sacrificed and samples of the ventral prostate lobes of each group were collected and tested for macroscopic and histopathological analysis. The remaining 10 animals of each group were left alive and followed for survival for more 10 weeks.

Results

Histopathological Analysis

Characterization of Prostatic Malignant Lesions

The low-grade adenocarcinoma was predominantly composed of well-formed acini showing discrete neoplasia and that resembled normal acini but with the absence of basal cells. The nuclei of neoplastic acini were bulky, oval and have prominent nucleoli.

In the intermediate grade adenocarcinoma, basal cells were also absent, neoplastic acini were sharp and began to merge.

High-grade adenocarcinoma was characterized by rare acini; the neoplastic cells were arranged in cords through the prostatic stroma.

TABLE PC-1
Frequency of prostate malignant lesions

| FREQUENCY OF | | | |
|---|---|---|---|
| MALIGNANT LESIONS | LOW | INTERMEDIATE | HIGH |
| TABLE PC-1 | GRADE | GRADE | GRADE |
| GROUP 1 | 20% | 40% | 40% |
| CANCER MNU | | | |
| GROUP 2 | 10% | 20% | 10% |
| IMMUNOMODULATOR | | | |
| 5 MG/KG | | | |
| GROUP 3 FLUTAMIDE | 10% | 30% | 10% |
| 10 MG/KG | | | |
| GROUP 4 | 20% | — | — |
| IMMUNOMODULATOR | | | |
| 2 MG/KG + | | | |
| FLUTAMIDE 2 MG/KG | | | |
| SEQUENTIALLY | | | |
| GROUP 5 | 10% | 10% | — |
| IMMUNOMODULATOR | | | |
| 2 MG/KG + | | | |
| FLUTAMIDE 2 MG/KG | | | |
| COMBINED | | | |

LEGENDS
Immunomodulator = proteic aggregate of ammonium and magnesium phospholinoleate-palmitoleate anhydride
Flutamide = Flutamide

TABLE PC-2
(%) SURVIVAL RATES

TABLE PC-2

| Survival | WEEKS | | | | | | | | | |
|---|---|---|---|---|---|---|---|---|---|---|
| Rates (%) | W1 | W2 | W3 | W4 | W5 | W6 | W7 | W8 | W9 | W10 |
| GROUP 1 | 100 | 80 | 80 | 70 | 70 | 70 | 30 | 30 | 20 | 20 |
| N = 10 | | | | | | | | | | |
| GROUP 2 | 100 | 100 | 100 | 80 | 80 | 60 | 60 | 50 | 50 | 50 |
| N = 10 | | | | | | | | | | |
| GROUP 4 | 100 | 100 | 100 | 100 | 100 | 100 | 100 | 100 | 100 | 100 |
| N = 10 | | | | | | | | | | |
| GROUP 5 | 100 | 100 | 100 | 100 | 100 | 100 | 100 | 100 | 100 | 100 |
| N = 10 | | | | | | | | | | |

### Results and Conclusion

All MNU-treated animals (Group 1) treated with saline developed prostate cancer (PCa).

In this group, the animals present 20% low, 40% intermediate and 40% high-grade adenocarcinomas (Table PC-1 Group 1).

The animals that used the immunomodulator (proteic aggregate of ammonium and magnesium phospholinoleate-palmitoleate anhydride) (Group 2), flutamide (Group 3) and combinations of the immunomodulator plus flutamide (Group 4 and Group 5) also presented lesions, but such lesions were totally distinct in grade and frequency of malignant lesions of the Group 1:

There was a reduction of the frequency of high-grade tumoral lesions in animals of the group that used Flutamide alone (Table PC-1 Group 3).

The most frequent malignant lesion observed in the animals of the Flutamide group (10 mg/kg) was intermediate grade adenocarcinoma (30%), followed by prostatic adenocarcinoma of low grade (10%) and (10%) high grade (Table PC-1 Group 3).

There was also a reduction of high-grade malignant lesions in the Immunomodulator—5 mg treated group (Group 5), which showed intermediate low-grade adenocarcinoma (10%), intermediate-grade carcinoma (20%) and high-grade carcinoma (10%).

In sharp contrast, the result of the use of the invention was remarkable:

The two groups of animals that used the combination of the immunomodulator (2 mg/k) and Flutamide (5 mg/kg), sequentially (group 4) or concomitantly (Group 5) shows 100% of reduction in the frequency of high-grade malignant lesions.

The group 4 shows 20% of low-grade lesions and the group 5 shows 10% of low-grade lesions and 10% of intermediate lesions, respectively. None high-grade malignant lesions were found in such groups.

Of note, smaller doses of compounds were used to provide a broader therapeutical effect that is wider and distinct from the effects of the isolated compounds, both the immunomodulator as well the anti-androgen therapy (flutamide.).

The survival rates (Table PC-2) are in accordance with the results shown in the Table PC-1.

Therefore, the claimed invention, that is, a combination of an immunomodulator (proteic aggregate of ammonium and magnesium phospholinoleate-palmitoleate anhydride) plus anti-androgen therapy (flutamide) is able to provide synergistic effects against prostate cancer and represents a clear improvement over the closest art.

### Treatment of Pancreatic Cancer

State of Art

Pancreatic cancer is considered one of the deadliest types of cancer. In US (2012), the incidence of new cases of pancreas cancer was 12.4 per 100,000 men and women per year. The number of deaths was 10.9 per 100,000 men and women per year.

According to NCI/SEER (National Cancer Institute/Surveillance, Epidemiology, and End Results Program), the pancreas cancer represents 3.0% of all new cancer cases in the U.S. In 2015 are expected 48,960 new cases with 40,560 deaths in US. Also according to NCI/SEER the percent survival in five years after diagnosis is around 7.2% of the patients.

Pancreas cancer is treated and/or managed by means of surgery (curative or palliative), chemotherapy and radiotherapy. Such therapies often are used in combination, which comprises its use previously, concomitantly, sequentially or simultaneously according the therapeutic strategy adopted.

Concerning chemotherapy to treat pancreatic cancer, the main drugs in use in the state of art are gemcitabine, the platinum derivates (cisplatin, carboplatin and oxaliplatin), fluorouracil (5 FU), docetaxel, paclitaxel and irinotecan that are used alone or combined. Of note, single-agent gemcitabine is a standard drug treatment for pancreatic cancer. Gemcitabine is also used in combinations with other drugs, including the above cited.

The rationale for use of combinations of drugs in treatment of cancer, including pancreatic cancer, is that combinations usually work better than single drugs because different drugs kill cancer cells in different stages of differentiation, due its distinct mechanism of action or potentiation of its anticancer effects.

Concerning pancreatic cancer, the best combination or regimen in the closest art is the regimen named FOLFIRINOX because includes the drugs fluorouracil (also known as 5-FU), leucovorin (folinic acid), irinotecan, and oxaliplatin. The drug Leucovorin (folinic acid) was introduced in this regimen aiming to minimize the toxic effects caused by the other components (5-FU). Patients who received the regimen called FOLFIRINOX, lived approximately 4 months longer than patients treated with gemcitabine alone (11.1 months compared with 6.8 months).

The main problem that impairs its use is severe toxicity for most of patients with pancreatic cancer and/or metastatic disease. Such toxic events include severe neutropenia, neuropathy and gastrointestinal problems. Neutropenia is the major concern, because most patients have a biliary stent. Biliary stents are common in patients with pancreatic cancer, because tumors that are formed in the head of the pancreas can often obstruct the bile duct, preventing it from feeding properly into the small intestine; a stent is used to alleviate the obstruction.

As widely known in the art, neutropenia, caused mainly by cytotoxic drugs, puts patients of cancer at increased risk for infections that can lead to sepsis, a potentially fatal blood infection.

Other emerging treatment is the combination of paclitaxel plus gemcitabine. An important study published in 2013 describes the results of a randomized clinical trial conducted in patients with metastatic pancreatic cancer that compared gemcitabine with a combination of gemcitabine plus albumin-bound paclitaxel (nab-paclitaxel).

In the cited clinical trial a total of 861 patients were randomly assigned to nab-paclitaxel plus gemcitabine (431 patients) or gemcitabine (430 patients). The survival rate was 35% in the nab-paclitaxel-gemcitabine group versus 22% in the gemcitabine group at 1 year. The most common adverse events of grade 3 or higher were neutropenia (38% in the nab-paclitaxel-gemcitabine group vs. 27% in the gemcitabine group), fatigue (17% vs. 7%), and neuropathy (17% vs. 1%). Febrile neutropenia occurred in 3% versus 1% of the patients in the two groups. (Von Hoff D. D. et al. Increased Survival in Pancreatic Cancer with nab-Paclitaxel plus Gemcitabine. N Engl J Med. 2013 Oct. 31; 369(18): 1691-170).

However, the efficacy of such combination is not consensual: In the United Kingdom, the National Institute for Health and Care Excellence (NICE), in a draft guidance issued in 2014, rejected such treatment due to concerns with side effects, efficacy, and relative costs of single-drug gemcitabine.

Taking in account the state of art as above resumed, that pointed to a urgent need of improvement over the available treatments, the immunomodulator (proteic aggregate of ammonium and magnesium phospholinoleate-palmitoleate anhydride) was evaluated in combinations with gemcitabine, 5-FU, taxanes (paclitaxel) and platinum derivates (cisplatin, carboplatin) irinotecan, using an appropriate animal model for the study of pancreatic cancer and evaluation of new treatments.

In addition, a small number of patients used the claimed invention in combination with FOLFORINOX regimen after surgery of pancreas. The remarkable therapeutic results are also described in details in this Specification.

Examples of Practical Use of the Invention in Pancreatic Cancer—Immunomodulator Combined with Gencitabine—Fluorouracil (5Fu)-Cisplatin—Oxaliplatin-Irinotecan

Experimental design for all combinations of the immunomodulator (proteic aggregate of ammonium and magnesium phospholinoleate-palmitoleate anhydride) plus chemotherapy drugs.

In a series of experiments were used 140 male Fisher 344 rats of 12 weeks of age and weighing about 200 g.

To induce the pancreatic cancer (CP), 130 rats were anesthetized with Xylazine Hydrochloride 2% (5 mg/kg i.m.; König, São Paulo, Brasil) and Ketamine Hydrochloride 10% (60 mg/kg, i.m.; Fort Dodge, Iowa, EUA) and conducted laparotomy to expose the head of pancreas.

After, a dissection was performed to separate the stomach and the transverse colon, followed by the identification of the biliopancreatic duct.

A 5 mm incision was performed parallel to the course of the biliopancreatic duct, avoiding its obstruction. In this incision site (between the duodenum and pancreas head) was deposited 200 μg of Dimethylbenzanthracene (DMBA—Sigma, St. Louis, Mo., EUA) dissolved in 200 μL of acetone).

The carcinogenic compound (DMBA) was maintained in the incision site by a purse string suture with a prolene 6-0 string. The number of animals treated with DMBA was 130. Additional 10 rats not treated with DMBA were also included to the experiment to provide baseline biochemical parameters (Group 1-Control Group). Another group of 10 animals was induced with DMBA and treated only with saline (Cancer Group-Group 2).

After 120 days of the surgery, all animals were submitted to ultrasound exams of the abdominal-pelvic cavity to check the presence of changes indicative of CP and posteriorly, they were subdivided in 10 groups (10 animals per group):

Control Group (Group 1): received intraperitoneal injections of 5 mL/kg of sterilizing saline solution 0.9%, three times a week for 10 consecutive weeks;

Cancer Group (Group 2): received the same treatment as Group 1;

Cancer+Immunomodulator Group (Group 3): received intraperitoneal injections of 6 mg/Kg of the Immunomodulator alone three times a week for 10 consecutive weeks;

Cancer+Gemcitabine Group (Group 4): received intraperitoneal injections of 100 mg/Kg of Gemcitabine one time a week for 10 consecutive weeks;

Cancer+Immunomodulator+Gemcitabine Group (Group 5): received sequentially intraperitoneal injections with the immunomodulator (3 mg/kg) three times a week) for 5 weeks and Gemcitabine (50 mg/kg) one time a week for 5 weeks;

Cancer+5 FU (Group 6): received intraperitoneal injections of 100 mg/kg of 5-FU one time a week for 10 consecutive weeks;

Cancer+Immunomodulator+5 FU (Group 7): received sequentially intraperitoneal injections with immunomodulator three times a week (3 mg/kg/5 weeks) and 5-FU (50 mg/kg) one time week for 5 weeks;

Cancer+Cisplatin (Group 8): received intraperitoneal injections of Cisplatin (50 mg/kg) three times a week for 10 weeks;

Cancer+Immunomodulator+Cisplatin (Group 9): received sequentially intraperitoneal injections with immunomodulator three times a week (3 mg/kg) for 5 weeks and Cisplatin (25 mg/kg) three times a week for 5 weeks;

Cancer+Oxaliplatin (Group 10): received intraperitoneal injections of Oxaliplatin (50 mg/kg) three times a week for 10 consecutive weeks;

Cancer+Immunomodulator+Oxaliplatin (Group 11): received sequentially intraperitoneal injections with immunomodulator three times a week (3 mg/kg) for 5 weeks and Oxaliplatin (25 mg/kg) three times a week for 5 weeks;

Cancer+Irinotecan (Group 12): received intraperitoneal injections of Irinotecan (40 mg/kg) three times a week for 10 consecutive weeks;

Cancer+Immunomodulator+Irinotecan (Group 13): received sequentially intraperitoneal injections with immunomodulator three times a week (3 mg/kg) for 5 weeks and Irinotecan (20 mg/kg) three times a week for 5 weeks.

The animals from all experimental groups received water and the same solid diet ad libitum (Nuvilab, Brazil) and were housed in individual boxes with solid floor, lined with wood shavings in bright room (12 h of light and 12 h dark) at controlled temperature (20 to 25° C.). The body weight of all animals was daily measured. After 10 weeks of treatment, pancreas samples of all rats were collected to perform histopathological analysis to evaluate efficacy against the malignancy.

In addition, the body weight of animals was monitored for evaluation of effects of treatments. Complete blood counts (Hemogram) were also performed in the beginning and the end of the experiment for controls and treated animals, for evaluation of toxicities and/or adverse events associated with the treatments, if any.

Blood samples of the all groups (Group 1 to Group 13) were obtained by puncturing the orbital plexus of the animals. Leukocyte counts were made in the peripheral blood of the treated and control animals, in the beginning and at the end of the experiment.

The global count of blood cells was performed by automated methods using a Coulter counter—STKS model.

Leukocyte counts were made in the peripheral blood of the treated and control animals, in the beginning and at the end of the experiment.

The specific and differential count of leukocytes was made in Giemsa-stained blood smears, (total 100 cells).

Histologic Analysis—Material and Methods

Pancreatic cancer samples from all animals of each experimental group (n=10) were collected and fixed in Bouin Liquid for 12 hours. After fixation, the tissues were washed in ethylic alcohol 70%, with posterior dehydration in an ascending series of alcohols. The fragments were diaphanized with xylene for 2 hours and enclosed in plastic polymers (Paraplast Plus, ST. Louis, Mo., EUA).

Then, the materials were sectioned in microtome Biocut 1130 (Reichert-Jung, Munique, Alemanha) with a thickness of 5 micrometers, stained with hematoxylin-eosin and photographed in the light microscope Nikon Eclipse Ni-U (Nikon, Tóquio, Japão) equipped with camera Nikon DS-RI-1 (Nikon, Tóquio, Japão).

The diagnosis of pancreatic lesions was based on morphological criteria.

Results: Histopathological Analysis

The results showed that pancreatic cancer animal model used was effective and induced tumors in 100% of animals. In the Cancer Group (TABLE P-1-Group 2) 60% of the animals showed neoplastic acini arranged in nests or cords that penetrated the stroma, characterizing infiltrating adenocarcinoma. Furthermore, 40% of animals in this group (Group 2) showed vacuole formations in atypical acinar cells, characterizing cystic pancreatic adenocarcinoma. (TABLE P-1).

Table P-1: Results

There was a reduction of 40% in the occurrence of tumors in the immunomodulator-treated group (Group 3) which shows 20% of the animals with normal histological recovery of the gland and 20% with chronic inflammation and fibrous stroma. The 60% remaining animals of this group (Group 3) showed infiltrating adenocarcinoma (TABLE P-1).

TABLE P-1
Percentage of histopathological changes of the pancreas of rats from
Control (Group 1), Cancer (Group 2) and Cancer + Immunomodulator:
6 mg/kg/ip/10 weeks (Group 3).

| | CONTROL | CANCER | |
|---|---|---|---|
| | GROUP | GROUP | IMMUNOMODULATOR |
| | Saline/ | Saline/ | 6 mg/kg/ip/ |
| | 10 weeks | 10 weeks | 10 weeks |
| | Groups | | |
| TABLE P-1 | Group 1 | Group 2 | Group 3 |
| Histopathology | N = 10 | N = 10 | N = 10 |
| Normal | 10 (100%) | — | 2 (20%) |
| Chronic | — | — | 2 (20%) |
| inflammation + | | | |
| Fibrous stroma | | | |
| Infiltrating | — | 06 (60%) | 6 (60%) |
| adenocarcinoma | | | |
| Cystic | — | 04 (40%) | — |
| adenocarcinoma | | | |

TABLE P-1
W-1 Average Body Weight of rats from CONTROL, CANCER and
IMMUNOMODULATOR groups during Treatment

| | | CANCER | IMMUNO- |
|---|---|---|---|
| TABLE P-1 W-1 | CONTROL | Saline/ | MODULATOR |
| Average Body | Saline/10 weeks | 10 weeks | 6 mg/kg/10 weeks |
| Weight/ | GROUPS | | |
| WEEKS OF | GROUP 1 | GROUP 2 | GROUP 3 |
| TREATMENT | N = 10 | N = 10 | N = 10 |
| Week 1 | 200 | 200 | 200 |
| Week 2 | 200 | 190 | 200 |
| Week 3 | 200 | 190 | 200 |
| Week 4 | 200 | 190 | 200 |
| Week 5 | 210 | 180 | 200 |
| Week 6 | 210 | 170 | 210 |
| Week 7 | 210 | 150 | 210 |
| Week 8 | 220 | 150 | 210 |
| Week 9 | 220 | 150 | 220 |
| Week 10 | 220 | 140 | 220 |

Table P-1 W-1 Results:

There was severe weigh loss in Group 2 (Cancer+saline) due to carcinogenic process. In contrast, the Group 1 (Control without cancer) and Group 3 (Cancer+immunomodulator) have not shown signs of weight loss. Notably the Group 3, treated with the immunomodulator alone, has shown a discrete weight gain, despite the presence of carcinogenic process.

TABLE P-1 H-1

| | Group 1 | Group2 | |
|---|---|---|---|
| | CONTROL | CANCER | Group 3 IMMUNO- |
| Table P1-H-1 | Saline/10 | Saline/10 | MODULATOR |
| HEMOGRAM | weeks | weeks p 2 | 6 mg/kg/10 weeks |
| Erythrocytes | 10 ± 2.5 | 9 ± 2.0 | 10 ± 2.0 |
| (10⁶/mm³) | | | |
| Leukocytes | 15.80 ± 4.8 | 14.2 ± 4.0 | 18.3 ± 2.2 |
| Lymphocytes × | 14.6 ± 3.9 | 12.8 ± 3.7 | 18.8 ± 4.1 |
| (10³/mm³) | | | |
| Neutrophils × | 0.95 ± 0.8 | 0.26 ± 0.9 | 1.8 ± 0.6 |
| (10³/mm³) | | | |
| Monocytes × | 0.1 ± 0.1 | 0.3 ± 0.1 | 0.2 ± 0.1 |
| (10³/mm³) | | | |
| Eosinophils × | 0.0 ± 0.0 | 0.0 ± 0.0 | 0.3 ± 0.1 |
| (10³/mm³) | | | |
| Basophils × | 0.1 ± 0.1 | 0.0 ± 0.0 | 0.0 ± 0.0 |
| (10³/mm³) | | | |

Table P-1-H-1—The results show that the combination of the immunomodulator was able to protect the animals against lymphopenia and neutropenia, despite the use of cytotoxic drug in association.

TABLE P-2
Percentage of histopathological changes of the pancreas of rats from
Cancer (Group 2-saline) and treated with gemcitabine 100 mg/kg/ip
(Group 4) and treated with the immunomodulator (3 mg/kg/ip./5 weeks) +
gemcitabine (50 mg/kg/ip./5 weeks - Group 5).

| | | | IMMUNO- |
|---|---|---|---|
| | | | MODULATOR |
| | CANCER | GENCITABINE | (3 mg/kg)/5 weeks + |
| | Saline/ | 100 mg/kg/10 | GENCITABINE |
| | 10 weeks | weeks | (50 mg/kg)/5 weeks |
| TABLE P-2 | Groups | | |
| Histopathology | Group 2 | Group 4 | Group 5 |
| Normal | — | 02 (20%) | 03 (30%) |
| Chronic | — | 01 (10%) | 05 (50%) |
| inflammation + | | | |
| Fibrous stroma | | | |
| Infiltrating | 06 (60%) | 06 (60%) | 01 (10%) |
| adenocarcinoma | | | |
| Cystic | 04 (40%) | 01 (10%) | 01 (10%) |
| adenocarcinoma | | | |

Table P-2: Results

There was a reduction of 30% in the occurrence of tumors in the 100 mg/kg gemcitabine-treated group (Group 4) which showed 20% of the animals with normal histological recovery of the gland and 10% with chronic inflammation and fibrous stroma. The 60% remaining animals of this group showed infiltrating adenocarcinoma and 10% showed cystic adenocarcinoma (Table P-2-Group 4).

There was a reduction of 80% in the occurrence of tumors in the group treated with the claimed invention (immunomodulator: 3 mg/kg/5 weeks+ gemcitabine: 50 mg/kg/5 weeks-Table P-2-Group 5) which showed 30% of the animals with normal histological recovery of the gland and 50% with chronic inflammation and fibrous stroma. The 10% remaining animals of this group showed infiltrating adenocarcinoma and 10% showed cystic adenocarcinoma (Table P-2-Group 5).

| | | | IMMUNO- |
|---|---|---|---|
| | | GEMCITABINE | MODULATOR |
| TABLE | CONTROL | 100 MG/KG/10 | (3 mg/kg)/5 weeks + |
| P-2 W-2 | Saline/ | WEEKS | GENCITABINE |
| Average Body | 10 weeks | Saline/10 weeks | (50 mg/kg)/5 weeks |
| Weight/ | GROUPS | | |
| WEEKS OF | GROUP 1 | GROUP 4 | GROUP 5 |
| TREATMENT | N = 10 | N = 10 | N = 10 |
| Week 1 | 200 | 200 | 200 |
| Week 2 | 200 | 190 | 200 |
| Week 3 | 200 | 190 | 200 |
| Week 4 | 200 | 180 | 200 |
| Week 5 | 210 | 180 | 200 |
| Week 6 | 210 | 170 | 200 |
| Week 7 | 210 | 170 | 190 |
| Week 8 | 220 | 170 | 185 |
| Week 9 | 220 | 160 | 185 |
| Week 10 | 220 | 160 | 185 |

Table P-2 W-2 Results:

There was weigh loss in Group 4 (Gemcitabine 50 mg). In contrast, the Group 1 (Control without cancer) has not shown signs of weight loss. The Group 5 (Immunomodulator 3 mg+Gemcitabine 50 mg) has shown only small weight loss (200 gr to 185 gr).

| | | | Group 5 |
|---|---|---|---|
| | Group 1 | Group 4 | IMMUNO- |
| | CONTROL | GEMCITABINE | MODULATOR |
| | Saline/ | 100 MG | (3 mg/kg)/5 weeks + |
| Table P2-H2 | 10 | MG/KG 10 | GEMCITABINE |
| HEMOGRAM | weeks | WEEKS | (50 mg/kg)/5 weeks |
| Erythrocytes | 10 ± 2.5 | 7 ± 2.0 | 8 ± 2.0 |
| (10⁶/mm³) | | | |
| Leukocytes | 15.80 ± 4.8 | 10.3 ± 4.0 | 17.3 ± 2.2 |
| Lymphocytes × | 14.6 ± 3.9 | 10.8 ± 3.7 | 17.8 ± 4.1 |
| (10³/mm³) | | | |
| Neutrophils × | 0.95 ± 0.8 | 0.40 ± 0.9 | 3.6 ± 0.6 |
| (10³/mm³) | | | |
| Monocytes × | 0.1 ± 0.1 | 0.0 ± 0.1 | 0.1 ± 0.1 |
| (10³/mm³) | | | |
| Eosinophils × | 0.0 ± 0.0 | 0.0 ± 0.0 | 0.3 ± 0.1 |
| (10³/mm³) | | | |
| Basophils × | 0.1 ± 0.1 | 0.0 ± 0.0 | 0.0 ± 0.0 |
| (10³/mm³) | | | |

Table P-2-H-2—

The results show that the combination of the immunomodulator was able to protect the animals against lymphopenia and neutropenia, despite the use of cytotoxic drug in association.

TABLE P-3
Percentage of histopathological changes of the pancreas of rats from
Cancer (Group 2-saline) and treated with 5 Fluorouracil (5FU)
100 mg/kg/ip/10 weeks (Group 6) and treated with Immunomodulator
3 mg/kg/ip/5 weeks + 5 FU 50 mg/kg/ip/5 weeks (Group 7).

| | | | IMMUNOMODULATOR |
|---|---|---|---|
| | CANCER | 5 FU | (3 mg/kg)/5 weeks + |
| | Saline/ | 100 mg/kg/ | 5 FU |
| | 10 weeks | 10 weeks | (50 mg/kg)/5 weeks |
| | N = 10 | N = 10 | N = 10 |
| TABLE P-3 | Groups | | |
| Histopathology | Group 2 | Group 6 | Group 7 |
| Normal | — | — | 03 (30%) |
| Chronic | — | 02 (20%) | 02 (20%) |
| inflammation + | | | |
| Fibrous stroma | | | |
| Infiltrating | 06 (60%) | 07(70%) | 03 (30%) |
| adenocarcinoma | | | |
| Cystic | 04 (40%) | 01 (10%) | 02 (20%) |
| adenocarcinoma | | | |

### Table P-3: Results

There was a reduction of 20% in the occurrence of tumors in the 100 mg/kg in 5 FU-treated group (Group 6) which showed 20% of the animals with chronic inflammation and fibrous stroma. The 70% remaining animals of this group showed infiltrating adenocarcinoma and 10% showed cystic adenocarcinoma (Table P-3-Group 6).

There was a reduction of 50% in the occurrence of tumors in the group treated with the claimed invention (immunomodulator: 3 mg/kg/5 weeks+5FU: 50 mg/kg/5 weeks-Table P-3-Group 7) which showed 30% of the animals with normal histological recovery of the gland and 20% with chronic inflammation and fibrous stroma. The 30% remaining animals of this group showed infiltrating adenocarcinoma and 20% showed cystic adenocarcinoma (Table P-3-Group 7).

| | | | IMMUNO- |
|---|---|---|---|
| | | | MODULATOR |
| TABLE | | 5-FU | (3 mg/kg)/5 weeks + |
| P-3 W-3 | CONTROL | 100 MG/KG/10 | 5FU |
| Average Body | Saline/10 weeks | WEEKS | (50 mg/kg)/5 weeks |
| Weight/ | GROUPS | | |
| WEEKS OF | GROUP 1 | GROUP 6 | GROUP 7 |
| TREATMENT | N = 10 | N = 10 | N = 10 |
| Week 1 | 200 | 200 | 200 |
| Week 2 | 200 | 190 | 200 |
| Week 3 | 200 | 190 | 200 |
| Week 4 | 200 | 180 | 200 |
| Week 5 | 210 | 180 | 200 |
| Week 6 | 210 | 175 | 200 |
| Week 7 | 210 | 175 | 190 |
| Week 8 | 220 | 160 | 190 |
| Week 9 | 220 | 160 | 185 |
| Week | 220 | 160 | 185 |
| 10 | | | |

### Table P-3 W-3 Results:

There was strong weigh loss in Group 6 (5 FU 50 mg). In contrast, the Group 1 (Control without cancer) has not shown signs of weight loss. The Group 7 (Immunomodulator 3 mg+Gemcitabine 50 mg) has only shown small weight loss (200 gr to 185 gr).

| | | | Group 7 |
|---|---|---|---|
| | | Group 6 | IMMUNO- |
| | Group 1 | 5-FU | MODULATOR |
| | CONTROL | 100 MG/KG/ | (3 mg/kg)/5 weeks + |
| TABLE P3-H-3 | Saline/10 | 10 | 5FU |
| HEMOGRAM | weeks | WEEKS | (50 mg/kg)/5 weeks |
| Erythrocytes | 10 ± 2.5 | 7.0 ± 3.0 | 8.0 ± 2.0 |
| (10⁶/mm³) | | | |
| Leukocytes | 15.80 ± 4.8 | 10.2 ± 4.0 | 17.3 ± 2.2 |
| Lymphocytes × | 14.6 ± 3.9 | 10.8 ± 3.7 | 18.8 ± 4.1 |
| (10³/mm³) | | | |
| Neutrophils × | 0.95 ± 0.8 | 0.1 ± 0.9 | 1.8 ± 0.6 |
| (10³/mm³) | | | |
| Monocytes × | 0.1 ± 0.1 | 0.1 ± 0.1 | 0.2 ± 0.1 |
| (10³/mm³) | | | |
| Eosinophils × | 0.0 ± 0.0 | 0.3 ± 0.0 | 0.2 ± 0.1 |
| (10³/mm³) | | | |
| Basophils × | 0.1 ± 0.1 | 0.0 ± 0.0 | 0.1 ± 0.0 |
| (10³/mm³) | | | |

### Table P-3 H-3

The results show that the combination of the immunomodulator was able to protect the animals against lymphopenia and neutropenia, despite the use of cytotoxic drug in association.

TABLE P-4
Percentage of histopathological changes of the pancreas of rats
from Cancer (Group 2) and treated with cisplatin 50 mg/kg/ip/10
weeks (Group 8) and treated with Immunomodulator 3 mg/kg/ip/5
weeks + cisplatin 25 mg/kg/ip/5 weeks (Group 9).

| | | | IMMUNOMODULATOR |
|---|---|---|---|
| | CANCER | CISPLATIN | (3 mg/kg)/5 weeks + |
| | Saline/ | 50 mg/kg/ | CISPLATIN (25 mg/kg)/ |
| TABLE P-4 | 10 weeks | 10 weeks | 5 weeks |
| Histopathology | N = 10 | N = 10 | N = 10 |
| Groups | Group 2 | Group 8 | Group 9 |
| Normal | — | — | 03 (30%) |
| Chronic | — | 01 (10%) | 01 (10%) |
| inflammation+ | | | |
| Fibrous stroma | | | |
| Infiltrating | 06 (60%) | 07 (70%) | 04 (40%) |
| adenocarcinoma | | | |
| Cystic | 04 (40%) | 01 (10%) | 02 (20%) |
| adenocarcinoma | | | |

Table P-4: Results

There was a reduction of 10% in the occurrence of tumors in the 50 mg/kg cisplatin-treated group (Group 8) which showed 10% of the animals with chronic inflammation and fibrous stroma. The 70% remaining animals of this group showed infiltrating adenocarcinoma and 20% showed cystic adenocarcinoma (Table P-3-Group 8).

There was a reduction of 40% in the occurrence of tumors in the group treated with the claimed invention (immunomodulator: 3 mg/kg/5 weeks+cisplatin: 25 mg/kg/5 weeks-Table P-4-Group 9) which showed 30% of the animals with normal histological recovery of the gland and 10% with chronic inflammation and fibrous stroma. The 40% remaining animals of this group showed infiltrating adenocarcinoma and 20% showed cystic adenocarcinoma (Table P-4-Group 9).

| | | | IMMUNO- |
|---|---|---|---|
| TABLE P-4 W-4 | | CISPLATIN | MODULATOR |
| Average Body | | 50 MG/KG/ | (3 mg/kg)/5 weeks + |
| Weight/ | CONTROL | 10 | CISPLATIN |
| WEEKS OF | Saline/10 weeks | WEEKS | (25 mg/kg)/5 weeks |
| TREATMENT | GROUP 1 | GROUP 8 | GROUP 9 |
| GROUPS | N = 10 | N = 10 | N = 10 |
| Week 1 | 200 | 200 | 200 |
| Week 2 | 200 | 190 | 200 |
| Week 3 | 200 | 190 | 195 |
| Week 4 | 200 | 185 | 195 |
| Week 5 | 210 | 185 | 195 |
| Week 6 | 210 | 175 | 195 |
| Week 7 | 210 | 175 | 190 |
| Week 8 | 220 | 160 | 190 |
| Week 9 | 220 | 160 | 190 |
| Week 10 | 220 | 160 | 190 |

### Table P-4 W-4 Results:

There was strong loss of weigh in Group 8 (Cisplatin 50 mg). In contrast, the Group 1 (Control without cancer) has not shown signs of weight loss. The Group 9 (Immunomodulator 3 mg+Cisplatin 25 mg) has shown only small weight loss (200 gr to 190 gr).

| | | | Group 9 |
|---|---|---|---|
| | | | IMMUNO- |
| | Group 1 | Group 8 | MODULATOR |
| | CONTROL | CISPLATIN | (3 mg/kg)/5 weeks + |
| TABLE P-4-H-4 | Saline/10 | 50 MG/KG/ | CISPLATIN |
| HEMOGRAM | weeks | 10 WEEKS | (25 mg/kg)/5 weeks |
| Erythrocytes | 10 ± 2.5 | 9.0 ± 2.0 | 9.5 ± 1.0 |
| (10⁶/mm³) | | | |
| Leukocytes | 15.80 ± 4.8 | 14.2 ± 4.0 | 16.3 ± 2.2 |
| Lymphocytes × | 14.6 ± 3.9 | 14.8 ± 3.7 | 14.8 ± 4.1 |
| (10³/mm³) | | | |
| Neutrophils × | 0.95 ± 0.8 | 0.21 ± 0.9 | 1.8 ± 0.6 |
| (10³/mm³) | | | |
| Monocytes × | 0.1 ± 0.1 | 0.2 ± 0.1 | 0.4 ± 0.5 |
| (10³/mm³) | | | |
| Eosinophils × | 0.0 ± 0.0 | 0.0 ± 0.0 | 0.0 ± 0.1 |
| (10³/mm³) | | | |
| Basophils × | 0.1 ± 0.1 | 0.0 ± 0.0 | 0.0 ± 0.0 |
| (10³/mm³) | | | |

Table P-4 H-4

The results show that the combination of the immunomodulator was able to protect the animals against lymphopenia and neutropenia, despite the use of cytotoxic drug in association.

TABLE P-5
Percentage of histopathological changes of the pancreas of
rats from Cancer (Group 2) and treated with oxaliplatin
50 mg/kg/ip/10 weeks (Group 10) and treated with
Immunomodulator 3 mg/kg/ip/5 weeks + oxaliplatin
25 mg/kg/ip/5 weeks (Group 11).

| | | | IMMUNO- |
|---|---|---|---|
| | | | MODULATOR |
| | | | (3 mg/kg)/5 weeks + |
| | CANCER | OXALIPLATIN | OXALIPLATIN |
| | Saline/ | 50 mg/kg/ | (25 mg/kg)/ |
| TABLE P-5 | 10 weeks | 10 weeks | 5 weeks |
| Histopathology | N = 10 | N = 10 | N = 10 |
| Groups | Group 2 | Group 10 | Group 11 |
| Normal | — | — | 03 (30%) |
| Chronic | — | 02 (20%) | 01 (10%) |
| inflammation + | | | |
| Fibrous stroma | | | |
| Infiltrating | 06 (60%) | 04 (40%) | 04 (40%) |
| adenocarcinoma | | | |
| Cystic | 04 (40%) | 04 (40%) | 02 (20%) |
| adenocarcinoma | | | |

Table P-5: Results

There was a reduction of 20% in the occurrence of tumors in the 50 mg/kg in oxaliplatin-treated group (Group 10) which showed 20% of the animals with chronic inflammation and fibrous stroma. The 40% remaining animals of this group showed infiltrating adenocarcinoma and 40% showed cystic adenocarcinoma (Table P-5-Group 10).

There was a reduction of 40% in the occurrence of tumors in the group treated with the claimed invention (immunomodulator: 3 mg/kg/5 weeks+oxaliplatin: 25 mg/kg/5 weeks-Table P-5-Group 11) which showed 30% of the animals with normal histological recovery of the gland and 10% with chronic inflammation and fibrous stroma. The 40% remaining animals of this group showed infiltrating adenocarcinoma and 20% showed cystic adenocarcinoma (Table P-4-Group 11).

| | | | IMMUNO- |
|---|---|---|---|
| TABLE P-5 W-5 | | | MODULATOR |
| Average Body | CONTROL | OXALIPLATIN | (3 mg/kg)/5 weeks + |
| Weight/ | Saline/ | 50 MG/KG/10 | OXALIPLATIN |
| WEEKS OF | 10 weeks | WEEKS | (25 mg/kg)/5 weeks |
| TREATMENT | GROUP 1 | GROUP 10 | GROUP 11 |
| GROUPS | N = 10 | N = 10 | N = 10 |
| Week 1 | 200 | 200 | 200 |
| Week 2 | 200 | 190 | 200 |
| Week 3 | 200 | 190 | 195 |
| Week 4 | 200 | 185 | 195 |
| Week 5 | 210 | 180 | 195 |
| Week 6 | 210 | 180 | 195 |
| Week 7 | 210 | 175 | 190 |
| Week 8 | 220 | 170 | 190 |
| Week 9 | 220 | 170 | 190 |
| Week 10 | 220 | 170 | 190 |

Table P-5-W-5 Results:

There was strong loss of weigh in Group 10 (Oxaliplatin 50 mg). In contrast, the Group 1 (Control without cancer) has not shown signs of weight loss. The Group 11 (Immunomodulator 3 mg+Oxaliplatin 25 mg) has shown only small weight loss (200 gr to 190 gr).

| | | | Group 11 |
|---|---|---|---|
| | | | IMMUNO- |
| | Group 1 | Group 10 | MODULATOR |
| | CONTROL | OXALIPLATIN | (3 mg/kg)/5 weeks + |
| Table P-5-H-5 | Saline/10 | 50 MG/KG/10 | OXALIPLATIN |
| HEMOGRAM | weeks | WEEKS | (25 mg/kg)/5 weeks |
| Erythrocytes | 10 ± 2.5 | 9.8 ± 2.0 | 10 ± 2.0 |
| (10⁶/mm³) | | | |
| Leukocytes | 15.80 ± 4.8 | 17.2 ± 4.0 | 18.3 ± 2.0 |
| Lymphocytes × | 14.6 ± 3.9 | 14.8 ± 3.7 | 17.8 ± 3.0 |
| (10³/mm³) | | | |
| Neutrophils × | 0.95 ± 0.8 | 0.5 ± 0.9 | 1.8 ± 0.6 |
| (10³/mm³) | | | |
| Monocytes × | 0.1 ± 0.1 | 0.2 ± 0.1 | 0.1 ± 0.1 |
| (10³/mm³) | | | |
| Eosinophils × | 0.0 ± 0.0 | 0.0 ± 0.0 | 0.0 ± 0.0 |
| (10³/mm³) | | | |
| Basophils × | 0.1 ± 0.1 | 0.0 ± 0.0 | 0.0 ± 0.0 |
| (10³/mm³) | | | |

Table P-5 H-5

The results show that the combination of the immunomodulator was able to protect the animals against lymphopenia and neutropenia, despite the use of cytotoxic drug in association.

TABLE P-6
Percentage of histopathological changes of the pancreas of
rats from cancer (Group 2) and treated with irinotecan
40 mg/kg/ip/10 weeks (Group 12) and treated with
Immunomodulator 3 mg/kg/ip/5 weeks + irinotecan
20 mg/kg/ip/5 weeks (Group 13).

| | | | IMMUNO- |
|---|---|---|---|
| | | | MODULATOR |
| | | | (3 mg/kg)/5 weeks + |
| | CANCER | IRINOTECAN | IRINOTECAN |
| | Saline/ | 40 mg/kg/ | (20 mg/kg)/ |
| TABLE P-6 | 10 weeks | 10 weeks | 5 weeks |
| Histopathology | N = 10 | N = 10 | N = 10 |
| Groups | Group 2 | Group 12 | Group 13 |
| Normal | — | — | 03 (30%) |
| Chronic | — | 03 (30%) | 03 (30%) |
| inflammation + | | | |
| Fibrous stroma | | | |
| Infiltrating | 06 (60%) | 03 (30%) | 03 (30%) |
| adenocarcinoma | | | |
| Cystic | 04 (40%) | 04 (40%) | 01 (10%) |
| adenocarcinoma | | | |

Table P-6: Results There was a reduction of 30% in the occurrence of tumors in the 40 mg/kg in irinotecan-treated group (Group 12) which showed 30% of the animals with chronic inflammation and fibrous stroma. The 30% remaining animals of this group showed infiltrating adenocarcinoma and 40% showed cystic adenocarcinoma (Table P-6-Group 12).

There was a reduction of 60% in the occurrence of tumors in the group treated with the claimed invention (immunomodulator: 3 mg/kg/5 weeks+irinotecan: 20 mg/kg/5 weeks-Table P-6-Group 13) which showed 30% of the animals with normal histological recovery of the gland and 30% with chronic inflammation and fibrous stroma. The 30% remaining animals of this group showed infiltrating adenocarcinoma and 10% showed cystic adenocarcinoma (Table P-6-Group 13).

| | | | IMMUNO- |
|---|---|---|---|
| TABLE P-6 W-6 | | IRINOTECAN | MODULATOR |
| Average Body | CONTROL | 40 MG/KG/ | (3 mg/kg)/5 weeks + |
| Weight/ | Saline/ | 10 | IRINOTECAN |
| WEEKS OF | 10 weeks | WEEKS | (20 mg/kg)/5 weeks |
| TREATMENT | GROUP 1 | GROUP 12 | GROUP 13 |
| GROUPS | N = 10 | N = 10 | N = 10 |
| Week 1 | 200 | 200 | 200 |
| Week 2 | 200 | 190 | 200 |
| Week 3 | 200 | 190 | 195 |
| Week 4 | 200 | 185 | 195 |
| Week 5 | 210 | 180 | 195 |
| Week 6 | 210 | 180 | 195 |
| Week 7 | 210 | 180 | 195 |
| Week 8 | 220 | 170 | 195 |
| Week 9 | 220 | 170 | 195 |
| Week 10 | 220 | 170 | 195 |

Table P-6-W-6 Results:

There was strong loss of weigh in Group 12 (Irinotecan 20 mg). In contrast, the Group 1 (Control without cancer) has not shown signs of weight loss. The Group 13 (Immunomodulator 3 mg+Irinotecan 20 mg) has shown only small weight loss (200 gr to 195 gr).

| | | | Group 13 |
|---|---|---|---|
| | | | IMMUNO- |
| | Group 1 | Group 12 | MODULATOR |
| | CONTROL | IRINOTECAN | (3 mg/kg)/5 weeks + |
| Table P-6-H-6 | Saline/10 | 40 MG/KG/10 | IRINOTECAN |
| HEMOGRAM | weeks | WEEKS | (20 mg/kg)/5 weeks |
| Erythrocytes | 10 ± 2.5 | 7.0 ± 1.0 | 8.0 ± 2.0 |
| (10⁶/mm³) | | | |
| Leukocytes | 15.80 ± 4.8 | 14.2 ± 4.0 | 18.3 ± 2.2 |
| Lymphocytes × | 14.6 ± 3.9 | 13.8 ± 3.7 | 18.8 ± 4.1 |
| (10³/mm³) | | | |
| Neutrophils × | 0.95 ± 0.8 | 0.16 ± 0.9 | 1.6 ± 0.6 |
| (10³/mm³) | | | |
| Monocytes × | 0.1 ± 0.1 | 0.3 ± 0.1 | 0.2 ± 0.1 |
| (10³/mm³) | | | |
| Eosinophils × | 0.0 ± 0.0 | 0.0 ± 0.0 | 0.3 ± 0.1 |
| (10³/mm³) | | | |
| Basophils × | 0.1 ± 0.1 | 0.0 ± 0.0 | 0.0 ± 0.0 |
| (10³/mm³) | | | |

Table P-6 H-6—

The results show that the combination of the immunomodulator was able to protect the animals against lymphopenia and neutropenia, despite the use of cytotoxic drug in association.

Pancreatic Cancer: Results and Discussion

The experimental results show that the use of the claimed invention, that is, the immunomodulator (proteic aggregate of ammonium and magnesium phospholinoleate-palmitoleate anhydride) combined with several anti-cancer agents (e.g. gemcitabine, 5 FU, cisplatin, gentamicin, irinotecan) was able to provide synergistic effects against pancreatic cancer, evaluated by means of histological analysis.

Such synergistic effects for the use of the invention (combination of substances) comprises a therapeutical anti-cancer effect that is different and wider than the therapeutical effect of the drugs considered alone (See Table P-1 to P-16).

The smaller toxicity provided by the use of the invention is proved by means of measurements of the evolution of mean body weight of treated animals (Table P-1 W1 to Table P-6-W6) and for the complete blood counts (CBC-Hemogram), that show a protective effect by the invention against neutropenia and lymphopenia (Table P-1-H1 to Table P-6-H6). As proved, the invention as claimed also protect the animals of cachexia that is caused by the cancer itself and compounded by the toxic effects of cytotoxic therapies used to fight the main disease (Table P-1 W1 to Table P-6-W6).

Examples of Practical Use of the Invention in Ovarian Cancer—Immunomodulator Combined with Cisplatin-Gemcitabine

Tumor Induction.

The animals (n=120) were anesthetized using 10% ketamine (60 mg/kg, i.p.,) and 2% xylazine (5 mg/kg, i.p.,) during the estrous phase, and a 2-cm-incision through the skin and abdominal muscles was performed, and the left ovaries were accessed after grasping the fat pad surrounding the organ. The left ovary was injected under the bursa with a single dose of 100 μg of DMBA (Sigma Chemical Co, St Louis, Mo.) dissolved in 10 μL of sesame oil, which was used as the vehicle (Hoyer et al. 2009), and was returned intact to the abdominal cavity. Sham-surgery was conducted on the right ovary by administering only the vehicle. Muscle and skin layers were closed using a 3-0 silk suture. Prophylactic treatment using antibiotic (105 units of benzylpenicillin potassium) was administered for the animals. Over the next 120 days, OC development was observed by size and volume.

Animals and Experimental Design.

One hundred twenty adult female Fischer 344 rats (60-days-old, weighing ±200 g) were used in the experiment. The rats were individually housed in polypropylene cages with laboratory-grade pine shavings as bedding and maintained under constant room temperature (23±1° C.) and lighting (12 h light/dark cycle, lights switched on at 6 a.m.). Filtered tap water and standard rodent chow (3074 SIF, Purina) were provided ad libitum. Immunomodulator is proteic aggregate of ammonium and magnesium phospholinoleate-palmitoleate anhydride. All animals were divided into six groups (n=20/group):

Group 1 (Control) OC: composed of rats that were induced with DMBA and received only saline solution vehicle (Nacl 0.9%) for 10 weeks as treatment;

Group 2 (OC+Immunomodulator): composed of rats that were induced with DMBA and received the immunomodulator (proteic aggregate of ammonium and magnesium phospholinoleate-palmitoleate anhydride) alone (5 mg/kg/ip) for 10 weeks as therapy;

Group 3 (OC+CIS): composed of rats that were induced with DMBA and received the platinum compound Cisplatin (4 mg/kg/ip) alone for 10 weeks as therapy;

Group 4 (OC+Immunomodulator+CIS): composed of rats that were induced with DMBA and received sequentially doses of Immunomodulator (2 mg/kg/ip) for 5 weeks and the platinum compound Cisplatin (2 mg/kg/ip) for 5 weeks as treatment.

Group 5 (OC+Gemcitabine): composed of rats that were induced with DMBA and received the compound Gemcitabine (4 mg/kg/ip) alone for 10 weeks as therapy;

Group 6 (OC+Immunomodulator+Gem): composed of rats that were induced with DMBA and received sequentially doses of Immunomodulator (2 mg/kg/ip) for 5 weeks and Gemcitabine (2 mg/kg) for 5 weeks as treatment.

Drugs and Schedule

After developing Ovarian Cancer (200-days-old),
the animals designated to receive only saline were administered i.p. doses of 0.20 mL of 0.9% (v/v) physiological saline (Group 1).
the animals designated to receive the immunomodulator (proteic aggregate of ammonium and magnesium phospholinoleate-palmitoleate anhydride) alone were administered i.p. with doses of 5 mg/kg b.w. dissolved in 0.20 mL of 0.9% (v/v) physiological saline (vehicle) at a final concentration of 5 mg/mL, during 10 weeks (Group 2).
Cisplatin-treated animals received two doses of 5 mg/kg, via i.p., by week (on monday and thursday) dissolved in 0.20 mL of vehicle, during 10 weeks (Group 3).
Immunomodulator+cisplatin-treated animals received Immunomodulator in two doses of 2 mg/kg, via ip, by week (on monday and thursday), for five weeks and Cisplatin in two doses of 2 mg/kg, via i.p., by week (on monday and thursday) dissolved in 0.20 mL of vehicle, for 5 weeks (Group 4).
gemcitabine-treated animals received two doses of 5 mg/kg, via i.p., by week (on monday and thursday) dissolved in 0.20 mL of vehicle, for 10 weeks (Group 5).
Immunomodulator+gemcitabine-treated animals received two doses of 2 mg/kg, via ip, by week (on monday and thursday), for five weeks and gemcitabine in two doses of 2 mg/kg, via i.p., by week (on monday and thursday) dissolved in 0.20 mL of vehicle, for 5 weeks (Group 6).

All single drugs were administered intraperitoneally twice a week for 10 consecutive weeks.

For the combination of immunomodulator (proteic aggregate of ammonium and magnesium phospholinoleate-palmitoleate anhydride) plus cisplatin and/or gemcitabine, after developing Ovarian Cancer (200-days-old), the animals were administered sequentially with each drug during 5 weeks (Table OC-1).

For the evaluation of survival rates, after the period of induction of ovarian cancer (200 days), all the animals were also followed up during the whole period of administration (10 weeks).

TABLE OC1
Drugs and Schedule

| Table OC-1 | WEEKS | | | | | | | | | |
|---|---|---|---|---|---|---|---|---|---|---|
| DRUGS | 1 | 2 | 3 | 4 | 5 | 6 | 7 | 8 | 9 | 10 |
| Group 1 - Cancer | x | x | x | x | x | x | x | x | x | x |
| Control (saline) | | | | | | | | | | |
| N = 20 | | | | | | | | | | |
| Group 2 | x | x | x | x | x | x | x | x | x | x |
| Immunomodulator | | | | | | | | | | |
| alone (5 mg/kg) | | | | | | | | | | |
| N = 20 | | | | | | | | | | |
| Group 3 | x | x | x | x | x | x | x | x | x | x |
| Cisplatin alone - 5 mg/kg | | | | | | | | | | |
| N = 20 | | | | | | | | | | |
| Group 5 | x | x | x | x | x | x | x | x | x | x |
| Gemcitabine | | | | | | | | | | |
| alone - 5 mg/kg | | | | | | | | | | |
| N = 20 | | | | | | | | | | |
| Group 4 | x | x | x | x | x | no | no | no | no | no |
| Immunomodulator | | | | | | | | | | |
| (2 mg/kg) + | | | | | | | | | | |
| Group 4 - Cisplatin | no | no | no | no | no | x | x | x | x | x |
| (2 mg/kg) | | | | | | | | | | |
| N = 20 | | | | | | | | | | |
| Group 6 | x | x | x | x | x | no | no | no | no | no |
| Immunomodulator | | | | | | | | | | |
| (2 mg/kg) + | | | | | | | | | | |
| Group 6 | no | no | no | no | no | x | x | x | x | x |
| Gemcitabine | | | | | | | | | | |
| (2 mg/kg) | | | | | | | | | | |
| N = 20 | | | | | | | | | | |

Table OC-1 Legends
X: period with administration of drug
no = period with no administration of drug

After the experimental procedures, all animals were anesthetized and euthanized for sample collection for histologic analysis and laboratorial analysis (blood counts).

Blood samples of the all groups (Group 1 to Group 6) were obtained by puncturing the orbital plexus of the animals. Leukocyte counts were made in the peripheral blood of the treated and control animals, in the beginning (200 days) and at the end of the experiment (200 days+10 weeks). See Table OC-3.

The global count of blood cells was performed by automated methods using a Coulter counter—STKS model.

Leukocyte counts were made in the peripheral blood of the treated and control animals, in the beginning and at the end of the experiment.

The specific and differential count of leukocytes was made in Giemsa-stained blood smears, (total 100 cells). (Table OC-3-A) and (Table OC-3-B).

All groups of animals were also monitored weekly for determination of evolution of body weight (Table OC-4) starting in the beginning of experiment (week 1) to the end of experiment (week 10). The results are expressed in average body weight.

### Experimental Results

TABLE OC-2
SURVIVAL RATES

| Table | | | | | | | | | |
|---|---|---|---|---|---|---|---|---|---|
| OC-2 | | | | | | | | | |
| SUR- | | | | | | | | | |
| VIVAL | 1 | 2 | 3 | 4 | 5 | 6 | 8 | 9 | 10 |
| RATES | week | week | week | week | week | week | week | week | week |
| Group 1 | 100% | 90% | 80% | 20% | 20% | 10% | 10% | 10% | 10% |
| Group 2 | 100% | 100% | 100% | 50% | 50% | 40% | 40% | 40% | 40% |
| Group 3 | 100% | 100% | 100% | 60% | 20% | 20% | 20% | 20% | 20% |
| Group 4 | 100% | 100% | 100% | 90% | 90% | 70% | 70% | 70% | 70% |
| Group 5 | 100% | 100% | 100% | 70% | 40% | 30% | 30% | 30% | 30% |
| Group 6 | 100% | 100% | 100% | 80% | 80% | 80% | 80% | 80% | 80% |

| | | | Group 11 |
|---|---|---|---|
| | Group 1 | Group 10 | IMMUNMODULATOR |
| | CONTROL | CISPLATIN | (2 mg/kg)/5 weeks + |
| Table 0C-3 (A) | Saline/10 | 5 MG/KG/10 | CISPLATIN |
| HEMOGRAM | weeks | WEEKS | (2 mg/kg)/5 weeks |
| Erythrocytes | 10 ± 2.5 | 9.8 ± 2.0 | 10 ± 2.0 |
| (10⁶/mm³) | | | |
| Leukocytes | 15.80 ± 4.8 | 17.2 ± 4.0 | 18.3 ± 2.0 |
| Lymphocytes × | 14.6 ± 3.9 | 14.8 ± 3.7 | 17.8 ± 3.0 |
| (10³/mm³) | | | |
| Neutrophils × | 0.95 ± 0.8 | 0.5 ± 0.9 | 1.8 ± 0.6 |
| (10³/mm³) | | | |
| Monocytes × | 0.1 ± 0.1 | 0.2 ± 0.1 | 0.1 ± 0.1 |
| (10³/mm³) | | | |
| Eosinophils × | 0.0 ± 0.0 | 0.0 ± 0.0 | 0.0 ± 0.0 |
| (10³/mm³) | | | |
| Basophils × | 0.1 ± 0.1 | 0.0 ± 0.0 | 0.0 ± 0.0 |
| (10³/mm³) | | | |

| | | Group 5 | Group 6 |
|---|---|---|---|
| | Group 1 | GEMTA- | IMMUNMODULATOR |
| | CONTROL | MICINE | (2 mg/kg)/5 weeks + |
| Table OC-3 (B) | Saline/10 | 5 MG/KG/10 | GENTAMICINE |
| HEMOGRAM | weeks | WEEKS | (25 mg/kg)/5 weeks |
| Erythrocytes | 10 ± 2.5 | 9.8 ± 2.0 | 10 ± 2.0 |
| (10⁶/mm³) | | | |
| Leukocytes | 15.80 ± 4.8 | 17.2 ± 4.0 | 18.3 ± 2.0 |
| Lymphocytes × | 14.6 ± 3.9 | 14.8 ± 3.7 | 17.8 ± 3.0 |
| (10³/mm³) | | | |
| Neutrophils × | 0.95 ± 0.8 | 0.5 ± 0.9 | 1.8 ± 0.6 |
| (10³/mm³) | | | |
| Monocytes × | 0.1 ± 0.1 | 0.2 ± 0.1 | 0.1 ± 0.1 |
| (10³/mm³) | | | |
| Eosinophils × | 0.0 ± 0.0 | 0.0 ± 0.0 | 0.0 ± 0.0 |
| (10³/mm³) | | | |
| Basophils × | 0.1 ± 0.1 | 0.0 ± 0.0 | 0.0 ± 0.0 |
| (10³/mm³) | | | |

TABLE OC-4
W-4- Average Body Weight of rats from groups 1 to 6 during treatment

| TABLE | | | | | | |
|---|---|---|---|---|---|---|
| OC-4 W-4 | | | | | | |
| Average Body | | | | | | |
| Weight/ | | | | | | |
| WEEKS OF | | | | | | |
| TREATMENT | GROUP 1 | GROUP 2 | GROUP 3 | GROUP 4 | GROUP 5 | GROUP 6 |
| Week 1 | 180 | 180 | 180 | 180 | 190 | 180 |
| Week 2 | 180 | 180 | 180 | 180 | 190 | 180 |
| Week 3 | 180 | 180 | 170 | 170 | 180 | 180 |
| Week 4 | 170 | 180 | 170 | 170 | 180 | 170 |
| Week 5 | 170 | 180 | 160 | 170 | 180 | 170 |
| Week 6 | 160 | 180 | 160 | 170 | 170 | 170 |
| Week 7 | 160 | 180 | 160 | 170 | 170 | 170 |
| Week 8 | 150 | 180 | 150 | 175 | 160 | 170 |
| Week 9 | 150 | 180 | 150 | 170 | 150 | 170 |
| Week 10 | 150 | 180 | 150 | 170 | 150 | 170 |

### Discussion of Results—Survival Rates

Analysis of data in Table OC-2 (Survival rates) shows that the use of the single-drug-immunomodulator (proteic aggregate of ammonium and magnesium phospholinoleate-palmitoleate anhydride) at dosage of 5 mg/kg resulted in a survival rate of 40% of the experimental animals (Table-OC-2 Group 2).

The single-drug-cisplatin used a dosage of 5 mg/kg resulted in a survival rate of 20% of the experimental animals (Table OC-2-Group 3).

The single-drug-gemcitabine used a dosage of 5 mg/kg resulted in a survival rate of 30% of the experimental animals (Table OC-2-Group 5).

In sharp contrast, the group with ovarian cancer (OC) that receives saline only shows 10% of survivals (Table OC-2 Group 1).

For the groups that was given the combination of drugs that constitute the present invention (Group 4 and Group 6), that is, the biological response modifier (proteic aggregate of ammonium and magnesium phospholinoleate-palmitoleate anhydride) at a dose of 2 mg/kg, associated to cisplatin at a dose of 2 mg/kg resulted in a survival rate of 70% in 10 weeks (Group 4-Table OC-2), which is much higher than the survival rate of 20% (Group 3-Table OC-2) obtained for the dose of 5 mg/kg of cisplatin (Table OC-2-Group 3) used without the presence of the immunomodulator.

The group that used the present invention, that is a combination of the immunomodulator (2 mg/kg Group 6-Table OC-2) associated with gemcitabine (2 mg/kg Group 6-Table OC2) resulted in a survival rate of 80% in 10 weeks days (Group 6-Table OC-2), which is much higher than the survival rate of 30% (Group 5-Table OC-2) obtained for the dose of 5 mg/kg of gemcitabine (Table OC-2-Group 5) used without the presence of the immunomodulator.

As it can be seen in the data from Table OC-2, the use of the invention, that is, the biological response modifier (proteic aggregate of ammonium and magnesium phospholinoleate-palmitoleate anhydride) used in the lower dose, that is, at the dose of 2 mg/kg, though associated to cisplatin also used at lower dosage of 2 mg/kg (Group 4-Table OC2), have made it possible to obtain a survival rate of 70% (Group 4-Table OC), compared to the 40% of survivors when the immunomodulator was administered alone at the dose of 5 mg/k, (Group 2-Table OC-2).

However, this 70% survival rate (Group 4-Table OC-2) obtained with the use of the invention is also much higher than the result obtained with the cytotoxic agent cisplatin when used at dosage of 5 mg/kg without the immunomodulator (proteic aggregate of ammonium and magnesium phospholinoleate-palmitoleate anhydride), which was only a 20% survival rate (Group 3-Table OC-2).

As it can also be seen in the data from Table OC-2, the use of the invention, that is, the immunomodulator (proteic aggregate of ammonium and magnesium phospholinoleate-palmitoleate anhydride) used in the lower dose, that is, at the dose of 2 mg/kg, though associated to gemcitabine also used at lower dose of 2 mg/kg (Group 6-Table OC 2), have made it possible to obtain a survival rate of 80% (Group 6-Table OC-2), compared to the 30% of survival rate for gemcitabine used alone at dosage of 5 mg/kg (group 5-Table OC-2).

Therefore, it is possible to conclude that the use of the invention is able to provide synergistic effects against the malignancy when the combination or association of the immunomodulator plus cisplatin (Table OC-2-Group 4) and the immunomodulator plus gemcitabine (Table OC-2-Group 6) is used for the treatment of ovarian cancer.

### Discussion of Results in Ovarian Cancer—Protection Against Cachexia and Other Toxic Effects of Cytotoxic Drugs Used Uin Combination

The group that used cisplatin alone (Group 3-Table OC-4: 180 gr week 1 and 150 gr at week 10) and gemcitabine alone (Group 5-Table OC-4: 190 gr week 1 and 150 gr week 10) shows severe weight loss.

The group that used the immunomodulator (proteic aggregate of ammonium and magnesium phospholinoleate-palmitoleate anhydride) alone not show weight loss (Group 2-Table OC 4: 180 gr at week 1 and 180 gr at week 10).

The group that used only saline also shows a severe weigh loss (180 grams/animal at week 1 and 150 grams/animal at week 10-Table OC-4 group 1) due the presence of the carcinogenic process.

In sharp contrast, the groups that used the claimed invention, that is group 4 (Group 4-Table OC 4: 180 gr week 1 and 170 gr week 10) and Group 6 (Group 6 Table OC 4; 180 gr week 1 and 170 gr week 10) only shows a very small weight loss despite the presence of carcinogenic process and the concomitant use of cytotoxic drugs.

Therefore, it's possible to conclude that the groups of animals that used the invention that is an association of the immunomodulator and cisplatin (Group 4-Table OC-4) and gemcitabine (Group 6-Table OC-4) were protected of the severe weight loss associated with the cancer process and compounded by the effects of the cytotoxic drugs used in combination (cachexia) and protected of lymphopenia and neutropenia (Table OC-3-A Hemogram and Table OC 3-B-Hemogram).

### Conclusions

The use of the claimed invention, that is the combination of drugs (immunomodulator plus cisplatin and/or gemcitabine) was able to show a remarkable synergistic activity against ovarian cancer measured by the number of survivals (Table OC-2-Group 4 and Group 6), using smaller doses of compounds, both the immunomodulator (anhydride used at 2 mg/kg) as well the platinum compound (cisplatin at 2 mg/kg) and the antimetabolic compound (gemcitabine at 2 mg/kg) without additional toxicity.

To the contrary, a deleterious event often associated with cancer and compounded by the most of the drugs and non-drug therapies used in the closest art to treat cancer (cachexia) measured in the experiment by the weight loss of treated animals (Table OC-4) was avoided for all the groups that used the claimed invention (Group 4-Table OC-4 and Group 6-Table OC-4). Also lymphopenia and neutropenia were avoided (Table OC-3-A Hemogram and Table OC 3-B-Hemogram).

### Pratical Example of Use of the Invention—Pancreatic Cancer—Human Subjects

Study of Case

The claimed invention was used to treat a male patient, 54 years old, suffering pancreatic cancer presenting tumoral mass on pancreas head identified by CT scan and PET scan performed immediately after the patient related severe abdominal pain in an interview with his physician.

The biopsy of pancreas tissue classified the malignancy as pancreatic adenocarcinoma affecting the head of pancreas. The tumoral marker (CA 19-9) showed 2300 units/ml when test was performed at the time of diagnosis.

The patient also presented previous Type 1 diabetes in the last 4 years before diagnosis, using insulin and presenting peritoneal implant of cancer at the time of diagnosis.

Previously to any drug treatment, the patient was submitted to surgery (pancreaticoduodenectomy) to remove all resectable tumors.

After the surgical procedure, the patient was treated with the claimed invention, comprising the use of FOLFIRONOX regimen (a combination of 5-FU, leucovorin, irinotecan, and oxaliplatin) during 10 weeks concomitantly with the immunomodulator (proteic aggregate of ammonium and magnesium phospholinoleate-palmitoleate anhydride) that was used in a dosage of 12.5 mg/square meter/2 times a week by intramuscular via, also during 10 weeks.

The immunomodulator was suspended and administered intramuscularly using sterile saline (NaCl 0.9%) as carrier for injections. The FOLFIRINOX regimen was used following its usual protocol.

The patient's follow up used CT scan, PET scan and tumoral marker (CA 19-9) performed every month during and after the use of the combination of the immunomodulator (proteic aggregate of ammonium and magnesium phospholinoleate-palmitoleate anhydride) plus FOLFIRINOX regimen.

### Results:

The clinical and laboratorial results concerning the use of the claimed invention were remarkable.

After 17 months of the end of procedures (surgery+folforinox regimen plus immunomodulator), the patient not shows any sign of tumoral process in pancreas or other organs, according to CT scan and PET scan.

The tumoral marker (CA 19-9) that initially showed 2300 units/ml at the time of diagnosis, decreased dramatically to 200 units/ml after 5 week of treatments and finally presenting, after the end of treatment, 30 units/ml (CA 19-9 normal values: 40 units/ml to 20 units/ml). CA 19-9 is the most widely used and best-validated marker for pancreatic cancer and is widely used for follow up after treatments. (Duffy M. C. et al. Tumor markers in pancreatic cancer: a European Group on Tumor Markers (EGTM) status report. Ann Oncol (2010) 21 (3): 441-447-Review).

Through the treatment the patient progressively showed weight gain. The body weight gain measured after 17 months of the end of treatment is around 10 kg. The patient remains alive and performing his professional and habitual activities without any limitation. Of note, the survival rates reported for FOLFIRINOX regimen when used alone are around 11 months.

Taking together, the results of imaging tests (CT scan, PET scan) and tumoral marker (CA 19-9) performed during and after the use of the claimed invention are strongly indicative that the cancerous process was eliminated.

Besides the use of surgery procedures, followed by the use of FOLFIRINOX regimen, the patient not showed relevant signs of toxicity or presence of deleterious events during and after the treatments with the invention.

The several complete blood counts (CBC) performed during and after the treatment with the claimed invention were within the normal parameters. Cachexia was remarkably absent during the treatment using the invention.

The patient had not presented neutropenia, lymphopenia, or any other signs of adverse events during and after the treatment. Only some episodes of nausea were reported during the use of FOLFIRINOX. Such events disappeared when the FOLFIRINOX regimen stopped.

Of note, the patient presented previous diabetes type 1, that is known for acting negatively in the immune status of persons suffering of this disease.

Despite the use of cytotoxic drugs (FOLFIRINOX regimen) and the presence of concomitant type 1 diabetes, the patient not showed any sign of neutropenia, febrile neutropenia nor episodes of opportunistic infections during and after the treatment.

The remarkable results of the example of practical use of the invention in a human subject suffering of pancreas cancer described above corroborates totally the synergistic effects that had arisen from the use of the claimed invention in the preclinical studies in pancreatic cancer (Table P-1 to Table P-6).

The example of use of the claimed invention in human subject also proves that the use of the invention provides a new solution that is able to treat cancer and concomitantly avoid or mitigate the comorbidities such as primary and secondary cachexia, neutropenia, febrile neutropenia and lymphopenia in experimental animals and human subjects, as described in the Specification and Claims.

Concluding, the results of the experiments in animals (Table P-1 to P-6) and human subjects proves that the claimed invention represents a clear improvement over the closest art in the treatment of cancer, including treatment of pancreatic cancer.

### Use of the Invention for Treatment of Prostate Cancer—Human Subjects

The invention was used to treat a patient male, 58 years old, with prostate carcinoma with presence of several bone metastasis. The biopsy revealed the presence of prostate adenocarcinoma hormone dependent.

Due the implantation of prostate tumor in the wall of rectum and presence of metastasis, the tumor was judged non-resectable in the moment of diagnosis.

Thus, adjuvant neo-chemotherapy was prescribed aiming to attack, reduce the tumor size if possible, and in consequence open the way for a subsequent curative or palliative surgery.

Ultrasonography and bone scan (bone scintigraphy) was used to evaluation of effect of the treatment. In addition, prostate specific antigen (PSA) was performed before, during and after the treatment.

The claimed invention was used as neo-adjuvant therapy aiming open the way for a future surgery and the immunomodulator (proteic aggregate of ammonium and magnesium phospholinoleate-palmitoleate anhydride) was applied by intramuscular via suspended in sterile saline for injection at 12.5 mg/square meter, 2 times a week and used in combination with anti-androgen therapy (Flutamide—tablet containing 200 mg) by oral via, 3 times a day. The duration of treatment was 6 months.

### Results

The claimed invention, that is, the combination immunomodulator (proteic aggregate of ammonium and magnesium phospholinoleate-palmitoleate anhydride) plus flutamide was well tolerated by the patient. No relates of toxicity or undesirable side events that could be linked with the use of the invention was reported. After one month of the combination therapy, the size of tumor reduced in 30%, showed in ultrasonography. The bone metastasis visualized by scintigraphy (bone scan) not shown alterations.

After 6 months of treatment, the tumoral size of the prostate tumor reduced in 50% and notably the bone metastasis was reduced in 40% in size.

The PSA that initially showed values of 8 ng/ml decreased to normal values (4 ng/ml) at the end of treatment (6 months).

The patient stopped the treatment and was directed to surgery to remove the remaining tumoral mass in the prostate and rectum.

The result of a treatment of a human subject suffering of metastatic prostate cancer described above confirms the remarkable and synergistic properties of the claimed invention as shown in the table PC-1 and PC-2.

### Practical Example of Use of the Invention for Treatment of Lung Cancer—Human Subjects

Study design: 6 (six) patients were enrolled.

Previous clinical condition: advanced-NSLC refractory to cisplatin plus carboplatin and docetaxel plus gemcitabine regimens.

Karnofsky performance scale for all patients at the beginning of clinical trial: 60%

### State of Art:

In the state of art, the standard first-line treatment for advanced non-small lung carcinoma (NSCLC) is a platinum-based two-drug combination regimen, such as cisplatin plus carboplatin.

For advanced and metastatic disease the drug Erlotinib, that is an inhibitor of EGFR (epidermal growth factor receptor), recently emerged as first-line or maintenance therapy.

The claimed invention was used to treat two (two) male patients with 50 and 54 years respectively, that presented advanced-stage non-small cell lung cancer (NSLC) not resectable with historic of previous surgery plus chemotherapy using cisplatin plus carboplatin—the standard first-line treatment for advanced NSCLC—that was used, but failed. Due the fail in the standard treatment, the patients were submitted to a combination of docetaxel plus gemcitabine, without favorable outcomes. Despite the use of two above-cited regimens, the disease progressed to metastatic presentation and the prognosis for both patients was very poor.

Due the impossibility to perform new surgeries and/or radiotherapy due contraindications and presence of advanced disease, the patients were submitted to the use of the claimed invention, that is, a combination of the immunomodulator (proteic aggregate of ammonium and magnesium phospholinoleate-palmitoleate anhydride) used intramuscularly at dosage of 12.5 mg/square meter, two shots a week suspended in sterile saline for injection, with a chemotherapy drug administered orally.

The drug elected for use in combination with the immunomodulator was an inhibitor of the epidermal growth factor receptor (Erlotinib) that is indicated as first line and for maintenance treatment of advanced-stage NSLC as well for the treatment of pancreatic cancer in combination with gemcitabine.

Thus, according the claimed invention, for combination with the immunomodulator (12.5 mg/square meter/2 times a week) the Erlotinib was administered orally at a dosage of 150 mg/day. The combination of drugs (Immunomodulator plus Erlotinib) was used continuously.

For comparative purposes 4 (four) other patients with advanced NSLC non-resectable using single-drug Erlotinib as maintenance therapy were followed up.

### Treatments Follow Up:

CT-Scan was used to follow up the progression of disease and treatments.

For all patients the Karnofsky performance scale was also applied to evaluate the patient's status and their clinical outcomes, if any.

### Results:

The two patients responded very well to the use of the claimed invention, that is immunomodulator (proteic aggregate of ammonium and magnesium phospholinoleate-palmitoleate anhydride) plus Erlotinib.

The patient of 50 years old survived for 26 months using the claimed invention with maintenance of a good status performance. The Karnofsky performance scale progressively progressed from 50% to 80% for 24 months. Meanwhile in the last 2 months before death the Karnofsky declined progressively to 60%.

The patient with 54 years old survived 20 months using the claimed invention. His Karnofsky scale progressed from 50% to 60% and this score was retained until this death.

For the patient of 50 years old, the CT scan showed reduction of 30% of the lesions and for the patient with 54 years old, the lesions showed reduction of 25%.

For the patients that used only single-drug Erlotinib, two patients showed 10% of regression of lesions as visualized by CT scan and the other two showed 15% and 18% of regression respectively. The Karnofsky performance scale of all patients using the single-drug Erlotinib was unchanged during the treatment.
The survival for the four patients was 10, 12, 15, 16 months after the beginning of treatment with single-drug Erlotinib, respectively.

### Conclusions:

Taking into an account the CT scan results and the clinical outcomes that includes survival and evaluations of performance (Karnofsky), the claimed invention, that is, the immunomodulator plus Erlotinib, has shown able to act synergistically in combination with an inhibitor of EGVR (Erlotinib) in advanced NSCLC disease.

These results provided by the use of the invention clearly represent an advance in relation to the previous art of treatment of lung cancer.

The same results also grants the use of the invention as maintenance therapy for use in lung cancer disease in general, that includes the treatment of advanced or metastatic-NSLC disease.

Taking into an account the improved outcomes provided by the use of the claimed invention as above explained and the remarkable results of preclinical studies (Table P-1 to Table P-6), it's possible to consider the combination of the immunomodulator (proteic aggregate of ammonium and magnesium phospholinoleate-palmitoleate anhydride) plus Erlotinib also for treatment of advanced-pancreatic cancer (Rocha Lima C. M. and Raes L. E. Erlotinib (Tarceva) for the Treatment of Non-Small-Cell Lung Cancer and Pancreatic Cancer. P T. 2009 October; 34(10): 554-556, 559-564).

Practical Example of the Use of the Invention for Reversal of Immunosuppression and Cachexia—Human Subjects

A practical example of the use of the present invention in cancer patients is provided, that is, the use of the biological response modifier (proteic aggregate of ammonium and magnesium phospholinoleate-palmitoleate anhydride) in a clinical trial, where the referred compound was associated to chemotherapy and radiotherapy protocols, in a comparative assessment (control group).

This example shall include data on the evolution of the main elements of the white blood cells, through blood tests (Table H), and also the evolution of body weight (Table M), to demonstrate in practice the innovative and remarkable properties of the invention in the prevention or treatment of immunosuppression and neutropenia, and also for the preventive, curative and palliative treatment of cachexia.

Objectives of the Clinical Trial:

(A) Assess in clinical trial on cancer patients receiving cytotoxic chemotherapy and/or radiotherapy, the effect of the invention on the leukocyte series, with the purpose of demonstrating its usefulness for the prevention and/or reversal of immunosuppression and neutropenia, and consequently minimizing or avoiding the occurrence of infections caused by cytotoxic chemotherapy and/or radiotherapy.

(B) Assess in clinical trial with patients receiving cytotoxic chemotherapy and/or radiotherapy, the effect of the invention on the evolution of body weight to demonstrate its usefulness in the preventive, curative or palliative treatment of cachexia or the anorexia-cachexia syndrome in the referred patients.

Experimental Protocol

A group of 85 cancer patients using chemotherapy and/or radiotherapy was selected for the use of the present invention, that is, the combination or association of the biological response modifier (proteic aggregate of ammonium and magnesium phospholinoleate-palmitoleate anhydride) with at least one other cancer treatment, that is, a combination or association with chemotherapy and/or radiotherapy (Group A and Group B) and compared to patients who received only the standard drugs in the state of the art (Group C).

The patients of the present study had solid tumors and have been given at least one type of cytotoxic/myelotoxic drug in association with other non-drug treatments (radiotherapy) or were treated with protocols of cytotoxic/myelotoxic drugs.

The patients treated by radiotherapy (RT) were given also at least one type of myelotoxic drug.

Associated drugs or protocols of chemotherapy drugs used in this clinical trial: EC (Etoposide+Cisplatin), VIP (Vinblastine+Ifosfamide), Cisplatin, VICE (Vincristine+Ifosfamide+Carboplatin+Etoposide), FCE (5-Fluoruacil+Cyclophosphamide+Epirubicin), DMCVB (Doxorubicin, Mitoxantrone, Cyclophosphamide, Vindesine and Bleomycin).

Clinical and laboratory follow-up: The patients of the three groups (A, B e C) were monitored through clinical tests, including weekly weight assessment and blood tests (complete blood count) with blood samples collected on the day immediately preceding the application of the biological response modifier, at the beginning of the cycles of treatment and at the end of the assessment period.

Hematological measurements: Leukocyte and erythrocyte counts were performed by using an automated counter.

Assessment of cachexia: The change in body weight is considered the most reliable marker for the analysis of the evolution of primary and secondary cachexia, including the assessment of the effect of specific drugs and therapies.

Therefore, all patients (Group A, Group B and Group C) were weighted on the Day immediately preceding (PI) the beginning of the application and then were weekly assessed until the end of the experiment, with the use of a precision mechanical scale.

The patients that participated in the clinical trial were divided into three groups:

Group A: Patients receiving chemotherapy (CT) and/or radiotherapy (RT) which was associated with the use of the (proteic aggregate of ammonium and magnesium phospholinoleate-palmitoleate anhydride) during the treatment period with CT and RT.

Number of patients: 25

Dose and dose regimen for all patients in Group A: 5 mg/m2/day in the form of suspension, using sterile saline solution as diluent (Nacl 0.9%) by intramuscular injection.

Duration of the protocol: 60 days

Group B: Patients that completed their initial cycles of chemotherapy (CT) and/or radiotherapy (RT) which was associated to the use of the biological response modifier (proteic aggregate of ammonium and magnesium phospholinoleate-palmitoleate anhydride) in the interval between the end and the beginning of the new cycles of CT and/or RT.

Number of patients: 30

Dose and dose regimen of the biological response modifier for all patients in Group B: 5 mg/m2/day in the form of suspension, using sterile saline solution as diluents (NaCl) at 0.9% by intramuscular injection.

Duration of the protocol: 60 days

Group C: The hematological and clinical parameters were obtained and analyzed, including the evolution of the body weight of 30 patients under cancer treatment, receiving the usual treatment that involves chemotherapy (CT) and/or radiotherapy (RT), without the use of the biological response modifier associated to the treatments, for comparison purposes (control group).

Number of patients: 30

Duration: 60 days

Practical Example—Results: Group A: Patients Using the Biological Response Modifier Associated to Chemotherapy and/or Radiotherapy

Group A—TOTAL LEUKOCYTES (percentages): Of the 25 patients, 18 (72%) had higher leukocyte values compared to the initial leukocyte values, 6 (24%) had decreased values compared to the initial values. The values of one patient (4%) remained unchanged.

Group A—NEUTROPHILS (percentages): Of the 25 patients, 18 (72%) had higher values compared to the initial neutrophil values, 6 (24%) had lower values compared to the initial values of neutrophils. The values of one patient (4%) remained unchanged.

Group A—LYMPHOCYTES: (percentages): Of the 25 patients, 16 (64%) showed higher values compared to the initial values of lymphocytes, 3 (12%) had lower values compared to the initial values. The values of six of the 25 patients (24%) remained unchanged.

Group A—ERYTHROCYTES (percentages): There were no significant changes in the levels of erythrocytes and hemoglobin during the trial, assessed in the beginning (TI) and at the end (TF) of the experiment.

Practical Example—Results: Group B: Patients with Associated Use (Sequential) of the Biological Response Modifier after the End of the Initial Cycles of Chemotherapy and Radiotherapy.

Results

Groups B—TOTAL LEUKOCYTES (percentages): Of the 28 patients, 25 (90%) had higher leukocyte values compared to the initial values and 3 patients (10%) had lower values compared to the initial values.

Group—NEUTROPHILS (percentages): Of the 28 patients, 22 (around 78%) had higher neutrophil values compared to the initial values. In 5 patients (17%) there were lower values compared to the initial values and in only 1 of the 28 patients (3%) the values of neutrophils remained unchanged.

Group B—LYMPHOCYTES: (percentages): Of the 28 patients, 24 (85%) had higher lymphocyte values compared to the initial values. In 4 of the 28 patients (15%) there were lower values compared to the initial values.

Group B—ERYTHROCYTES (percentages): No significant changes in the levels of erythrocytes and hemoglobin were detected during the trial, assessed in the beginning (TI) and at the end (TF) of the experiment.

Practical Example—Results: Group C: Patients Using Chemotherapy (Ct) and/or Radiotherapy (Rt) not Associated to the Biological Response Modifier.

Results

Groups C—TOTAL LEUKOCYTES (percentages): Of the 30 patients, 3 (10%) had higher values of leukocytes compared to the initial values and 21 patients (70%) had lower values compared to the initial values. The leukocyte values of de 6 (20%) patients remained unchanged.

Group C—NEUTROPHILS (percentages): Of the 30 patients, 28 (93%) had lower values of neutrophils compared to the initial values, and of these patients, 4 (14%) had severe neutropenia, that is, a neutrophil count of less than 900 per cubic millimeter accompanied by fever (febrile neutropenia), though without the identification or location of the infectious focus. The neutrophil values of 2 patients (7%) remained unchanged.

Group C—LYMPHOCYTES: (percentages): Of the 30 patients, 4 (13%) had higher values of lymphocytes compared to the initial values. In 21 of the 30 patients (70%) there were lower values compared to the initial values. The lymphocyte values of 5 patients (17%) remained unchanged.

ERYTHROCYTES (percentages): No significant changes were found in the levels of erythrocytes and hemoglobin during the trial, assessed in the beginning (TI) and at the end (TF) of the experiment.

Episodes of Febrile Neutropenia Reported During the Observation Period

Of the 30 patients in Group C, 4 patients, all male individuals, (14%) had severe neutropenia, that is, a neutrophil count of less than 900 cubic millimeter and accompanied by fever (febrile neutropenia), though without the identification or location of the infectious focus.

Episodes of Infection Reported During the Observation Period:

Of the 30 patients in Group C, 3 of them, all male individuals, (10%) had episodes of s herpes simplex (HSV-1) of moderate intensity and 2 female patients (around 7%) had fungal infection of moderate intensity caused by candida sp. in the genital region, that is, 17% of the total number of these patients had episode of infection by opportunistic agents (Table H-Group C) during the observation period.

Treatment of the Episodes of Neutropenia and Infection

The patients with febrile neutropenia (Group C) were treated with the standard procedure, that is, prophylaxis using empirical intravenous antibiotic therapy.

The patients with herpes simplex (HSV-1) (Group C) were treated with acyclovir (injectable) and the patients with fungal infection were treated with Fluconazole.

TABLE H:

| Evolution of the | | | | % of patients |
|---|---|---|---|---|
| leukocyte values | | | | with gain or |
| per group of | Final values | Final values | | maintenance of |
| patients (Values | higher than | lower than | Final values | the leukocyte |
| in %) | initial values | initial values | unchanged | values |
| TOTAL | Group A: 72% | Group A: 24% | Group A: 4% | Group A: 76% |
| LEUKOCYTES | Group B: 90% | Group B: 10% | Group B: 0% | Group B: |
| | Group C: 10% | Group C: 70% | Group C: 20% | 100% |
| | | | | Group C: 30% |
| NEUTROPHILS | Group A: 72% | Group A: 24% | Group A: 4% | Group A: 76% |
| | Group B: 78% | Group B: 17% | Group B: 3% | Group B: 81% |
| | Group C: 0% | Group C: 93% | Group C: 7% | Group C: 7% |
| LYMPHOCYTES | Group A: 64% | Group A: 12% | Group A: 24% | Group A: 88% |
| | Group B: 85% | Group B: 15% | Group B: 0% | Group B: 85% |
| | Group C: 13% | Group C: 70% | Group C: 17% | Group C: 30% |
| FEBRILE | Group A: 0% | | | |
| NEUTROPENIA | Group B: 0% | | | |
| (%) | Group C: 14% | | | |
| EPISODES OF | Group A: 0% | | | |
| IDENTIFIED | Group B: 0% | | | |
| INFECTION | Group C: 17% | | | |

EVOLUTION OF BODY WEIGHT-GROUP A: Of the 25 patients, 15 (60%) showed positive change in body weight measured after 60 days compared to the initial weight. In 4 patients (16%) there was weight loss compared to the initial body weight. In 6 patients (24%), the weight remained unchanged.

EVOLUTION OF BODY WEIGHT-GROUP B: Of the 30 patients, 15 (50%) showed positive change in body weight measured after 60 days compared to the initial weight. In 2 patients (approximately 7%) there was a weight reduction compared to the initial weight. In 13 patients (approximately 43%), the weight remained unchanged.

EVOLUTION OF BODY WEIGHT-GROUP C: Of the 30 patients, 26 (approximately. 87%) had weight loss compared to the initial weight. In 4 (approximately 13%) patients the weight remained unchanged. None of the patients in this group gained weight compared to the initial values.

In GROUP C, 6 patients (20%) had weight loss greater than 10% compared to the initial values, which can be considered a criterion that characterizes the presence of cachexia or its aggravation.

| | | | | | Percentage |
|---|---|---|---|---|---|
| TABLE M- | | | | | of patients |
| Weight | | | | Percentage | with weight |
| evolution by | Final body | Final body | Final body | of patients | loss greater |
| groups of | weight | weight | weight | with weight | than 10% in |
| patients | greater than | lower than | equal to | gain or | the |
| (values in | initial body | initial body | initial body | weight | observation |
| %) | weight | weight | weight | maintenance | period |
| Group A | 60% | 16% | 24% | 84% | 0% |
| Group B | 50% | 7% | 43% | 93% | 0% |
| Group C | 0% | 87% | 13% | 13% | 20% |

Discussion of Results:

The use of the present invention provides remarkable results in clinical practice, and it has been demonstrated in animal experiments (Table L) and clinical trials in humans (Table H) that the use of the present invention, that is, the association or combination of the biological response modifier specially selected to be part of this invention (proteic aggregate of ammonium and magnesium phospholinoleate-palmitoleate anhydride) with other drugs or chemotherapy known to be cytotoxic and depressors of the myeloid series can correct and/or counterbalance its effects.

In the blood test of the animals (Table L) and in that of humans (Table H) that used the invention, not only an increased number of neutrophils, but also of lymphocytes, which are important components of the immune system that protect the body against invasion by infectious agents and also against cancers were found.

Another important observation concerning the purposes of the present report is that the use of this invention has produced a significant difference in the percentage of reported cases of febrile neutropenia and infection that affected the patients of group C, who did not use the invention, or else, of the total patients in Group C (100%), 14% were found to have febrile neutropenia and 17% of the patients in this group had one episode of infection characterized throughout the 60 days of treatments and observation.

Notably and surprisingly, compared to Group C, none of the patients who were treated with the invention (Table H-Group A and Group B) had severe neutropenia, febrile neutropenia or opportunistic infection during the period (60 days) of the trial, although 24% of the patients in Group A had lower values of neutrophils (Group A-Table H) than those in the beginning of the treatment and 17% of the patients of Group B had shown lower values of neutrophils (Group B-Table H) than those observed in the beginning of the clinical trial.

These data show that the use of the invention, through the presence of the biological response modifier (proteic aggregate of ammonium and magnesium phospholinoleate-palmitoleate anhydride) is able to counterbalance in practice the immunosuppressive effects of the treatments used in combination or association, that is, cytotoxic chemotherapy and radiotherapy combined with chemotherapy (Table H-Group A and Group B).

Additionally, the use of the invention in clinical practice has another advantage, which is the ability to stimulate the production of lymphocytes when compared to the use of other current therapeutic agents in the state of the art, and used in combination or association with chemotherapy and/or radiotherapy treatments to revert or minimize neutropenia, the so-called myeloid series stimulating factors (C-CSF, GM-CSF) that have the ability to act in progenitor cells of the bone marrow to produce neutrophils. However, no cases of increase in the number of lymphocytes in the peripheral blood related to its use are reported in the state of the art.

One possible theoretical possibility suggested by the proven ability of connection of the biological response modifier to toll-like cell receptors, the so-called TLR-2 and TLR-4 (Table T-1 and Table T-2) is that the action of the biological response modifier (proteic aggregate of ammonium and magnesium phospholinoleate-palmitoleate anhydride) that is wider than that of myeloid series stimulating factors (C-CSF, GM-CSF) that is, the biological response modifier (proteic aggregate of ammonium and magnesium phospholinoleate-palmitoleate anhydride) might activate immune cells endowed with toll-like receptors (TLRs), such as macrophages and dendritic cells, which, in turn, would trigger the mechanisms that induce the stimulus to the production and differentiation of neutrophils from bone marrow precursors, as well as to lymphocyte proliferation and differentiation from the precursor cells in the bone marrow.

Another theoretical possibility to explain the additional capacity to induce lymphocytosis of the invention can be based on the proven ability of the biological response modifier (proteic aggregate of ammonium and magnesium phospholinoleate-palmitoleate anhydride) to attach itself or bind to toll like cell receptors (TLR-2 and TLR-4-Table T-1 and Table T-2), and once this binding is effected, the referred dendritic cells and macrophages are activated. Dendritic cells and macrophages are known to have these types of cell receptors in their surface and also antigen presenting cells that present antigens to T lymphocytes (T-4 lymphocytes or T-Helper), triggering the immune processes responsible for the differentiation and proliferation of the lymphocytes, elements extremely important in the fight against infections and cancer.

Regardless of the possible mechanisms of action, the effect or usefulness of any invention must be proven in practice and the clinical trial performed with humans has demonstrated in practice the remarkable effects of the present invention, which had already been demonstrated in animal experiments (Table L) of reverting or minimizing immunosuppression and neutropenia when administered to patients under cancer treatment using chemotherapy drugs (known to be myelotoxic) and radiotherapy (known to be immunosuppressive).

The adverse effects of chemotherapy and radiotherapy on the white blood cells evaluated in these patients through laboratory blood tests were avoided and/or reverted, since a marked increase in the number of neutrophils and lymphocytes was observed in the blood tests of patients of the groups (Table H-Group A and Group B) that used the present invention, that is, the biological response modifier (proteic aggregate of ammonium and magnesium phospholinoleate-palmitoleate anhydride) combined or associated to chemotherapy and radiotherapy compared to group (Table M-Group C) that did not use the present invention.

This laboratory effect on the white blood cells in patients that used the invention, that is, the biological response modifier (proteic aggregate of ammonium and magnesium phospholinoleate-palmitoleate anhydride) associated to chemotherapy and radiotherapy was also followed by remarkable and beneficial effects, since the episodes of febrile neutropenia and infection did not occur in the groups that used the invention (Group A and Group B-Table M) compared to the group (Group C-Table M) that did not use the invention.

The remarkable ability of the present invention constituted by the biological response modifier, in combination or association, of providing a preventive, curative or palliative treatment for immunosuppression and neutropenia caused by the use of cytotoxic and/or myelotoxic drugs was proven for instance by the examples of experimental tests in animal models (Table P1-H-1 to Table P-6-H6) and also by the effects in humans (Table M). This new practical application incorporated or provided by the present invention can by no means be considered a simple or obvious consequence of the state of the art.

Hence, the invention, that is, the biological response modifier (proteic aggregate of ammonium and magnesium phospholinoleate-palmitoleate anhydride) can be used in combination or association to prior, concomitant or subsequent treatment with various cytotoxic and/or myelotoxic compounds, drugs or treatments, which can be selected empirically at the time of treatment planning in practice, or even during the treatment, by adding, besides the increased anticancer effect conferred to the association or combination, the remarkable ability of treating immunosuppression and neutropenia in a preventive, curative or palliative character.

Also, when the invention is used in the manufacture of new drugs, in the form of associations or combinations, the presence of the biological response modifier (proteic aggregate of ammonium and magnesium phospholinoleate-palmitoleate anhydride) in the composition of such formulations, shall confer to the association or combination of drugs that include the invention, the same properties, that is, besides maximizing the anticancer effect of the association or combination, the invention shall concomitantly prevent, revert or minimize the immunosuppressive effect and/or neutropenia of any cytotoxic and/or myelotoxic drugs selected for use in association or combination with these new drugs.

Treatment of Cachexia-Discussion and Extrapolation of Data from the Clinical Trial (Table M).

Moreover, rapid weight loss, a sign usually associated to the presence of the malnutrition-cachexia syndrome or to the beginning of its manifestation or aggravation, was clearly prevented in the groups (Group A and Group B-Table M) compared to the control groups (Group C-Table M) during the experiment period.

On the contrary, in a remarkable way, 60% of the patients in the groups that used the invention (Group A-60%-Table M) and 50% (Group B-50%-Table M) had weight gain compared to the initial weight. Only 16% of these patients (Group A-Table M) and 7% (Group B-Table M), respectively, had a final body weight lower than the initial body weight. However, none of these patients had weight loss greater than 10% of the body weight compared to the initial weight in the beginning of the clinical trial.

In marked contrast with the group of patients subjected only to chemotherapy and/or radiotherapy without the use of the present invention (Group C-Table M), that is, of the biological response modifier used in association or combination (proteic aggregate of ammonium and magnesium phospholinoleate-palmitoleate anhydride), it can be seen in Table M that 87% of these patients had a final weight lower than the initial weight (Group C-Table M) (Group C-Table M) and finally 20% of these patients (Group C-Table M) were found to have a weight loss greater than 10% of the body weight compared to their initial weight, which characterizes the occurrence of cachexia.

None of the patients in Group C (Group C-Table M) had weight gain compared to the weight in the beginning of the experiment, and only 13% of these patients (Group C-Table M) maintained the initial body weight.

As it can be seen in Table M regarding weight gain or maintenance, with both parameters assessed in comparison with the initial levels, 84% of the patients in Group A (Group A-Table M) and 93% of the patients in Group B (Group B-Table M) maintained their weight or gained weight, whereas for Group C (Group C-Table M), only 13% of the patients maintained their initial body weight.

It should be noted that patients in all groups were being treated with protocols involving the combination of chemotherapy drugs and/or radiotherapy, with at least one anticancer drug, that is, all patients were receiving cytotoxic drugs, which could cause or aggravate, by local or systemic aggression, organic tissues and/or processes, thus contributing to additional weight loss in patients and because of the aggravation of cachexia (secondary cachexia).

In view of these remarkable results, it can be seen that the use of the invention prevented the weight loss that characterizes cachexia or its aggravation, which usually occurs with patients in cancer treatment and because of the drugs administered to treat this systemic disease, and thus, this invention can also be used in clinical practice for this purpose, that is, for the preventive, curative or palliative treatment of primary and secondary cachexia.

### Table M and Table H—Patients Follow Up—Survival—Two Years

The patients that are enrolled in the clinical trial above described were followed up for survival in the period of 2 years after the end of clinical trial.

For the group A, that is, patients that used the immunomodulator (proteic aggregate of ammonium and magnesium phospholinoleate-palmitoleate anhydride), combined with QT and RT, 15 patients of the 25 enrolled were still alive 2 years after the end of clinical trial.

For the Group B, that is, patients that used sequentially the immunomodulator (proteic aggregate of ammonium and magnesium phospholinoleate-palmitoleate anhydride), after the end of their cycles of QT and RT, 14 patients of the 30 enrolled remained alive 2 years after the end of clinical trial.

For the group C, that is patients that not used the immunomodulator combined with QT+RT, 10 of the 30 enrolled were still alive 2 years after the end of clinical trial. This numbers provide a strong indication that the combination of the immunomodulator (proteic aggregate of ammonium and magnesium phospholinoleate-palmitoleate anhydride), and QT+RT was able to provide better rates of survival for the groups of patients that used the invention (Groups A and B), when compared with the number of survivors that not used the claimed invention (Group C).

In addition, the new data concerning the survival rates of patients enrolled in clinical trial corroborates the remarkable results arisen from experiments using animal models for the study of lung cancer, pancreatic cancer, ovarian cancer and prostate cancer.

Therefore, the combined results arisen from the use of the invention in animals and human subjects, such as the new results of the clinical trial above described, prove that the invention is able to provide synergistic effects against the cancer and simultaneously acting against deleterious events related with the cancer and therapies, such as cachexia, neutropenia and lymphopenia.

Problems and Obstacles in the Treatment of Precancerous Lesions—Need for Improvements

Given the importance of early treatment to prevent the development of precancerous lesions to cancer and the continuing need for improvement in the treatments of these conditions of great clinical importance, some practical examples of the invention, that is, of the use of the biological response modifier as a new therapeutic option, and, thus, not known in the state of the art for the treatment of the aforementioned conditions, will be provided.

The biological response modifier (proteic aggregate of ammonium and magnesium phospholinoleate-palmitoleate anhydride) is reported in the state of the art as possessing curative properties in cancer, ie, after the disease has manifested itself (PI-0305373-3 Ser. No. 10/978,683, EPA 0426250.3.2405).

However, there is no report in the state of the art involving its use as a preventive therapy for cancer, i.e. to treat the so-called precancerous lesions, either alone or in the form of associations or combinations with other non-drug treatments.

The link between chronic inflammation and cancer has long been recognized in the state of the art, that is, the important role played by reactive inflammatory processes in the modification of a normal cell to cancer cell (carcinogenesis), and given the therapeutic effects obtained in clinical trials for events (cachexia) that can also be related to reactive inflammation, as one of its various causes, and new experimental data demonstrating the ability of the biological response modifier (proteic aggregate of ammonium and magnesium phospholinoleate-palmitoleate anhydride) of binding to toll-like cell receptors (U.S. application Ser. No. 13/516,628), and which indicate a role played by this compound in cell repair and regeneration processes, this compound has been deliberately selected as part of the present invention for the preventive treatment of cancer when the damage or carcinogenic factor is related to cell transformation associated to carcinogenic factors and/or agents, as will be extensively explained in the present report. This new practical application incorporated or provided by the present invention can by no means be considered a simple or obvious consequence of the state of the art.

Therefore, in order to demonstrate the applicability or usefulness of the compound, a biological response modifier (proteic aggregate of ammonium and magnesium phospholinoleate-palmitoleate anhydride) for the purposes of the present invention, the action of the referred compound was evaluated, in clinical practice, through an appropriate experimental model, on inflammatory and carcinogenic processes and also on the process of transformation of cell dysplasia into cancer, aiming to obtain its practical use as a preventive therapy for cancer, according to one of the purposes of the present invention.

Subsequently, after a brief explanation on models for experimental studies on carcinogenesis and on the treatment of precancerous lesions in the state of the art, some practical examples of the use of the present invention will be provided. These examples are produced for the sole purpose of illustrating the state of the art and do not intend to limit the scope of the present invention.

Experimental Models for Carcinogenesis

Since most precancerous lesions related to the aggressive action of external agents such as solar radiation, chemical substances such as alcohol and tobacco are located in the external (skin) or internal (gastrointestinal tract, bladder) epithelial tissues, it is important to choose the appropriate experimental model in order to reproduce as accurately as possible the situations that occur in practice.

In order to evaluate, in clinical practice, the effectiveness of the biological response modifier for the treatment of precancerous lesions caused or induced by aggressive agents the experimental model involving chemically induced carcinogenesis in hamsters (Mesocriatus auratus), using the carcinogenic compound called 7,12-dimethilbenz (a) anthracene or else dimethyl benzanthracene, or the abbreviation DMBA in the state of the art.

The compound called DMBA, named 7, 12-dimethylbenz (a) anthracene (dimethylbenzanthracene-DMBA) according to IUPAC standards. Molecular formula:

(C20H16), CAS (57-97-6 Y), PubChem (6001) is a chemical compound belonging to the class of polycyclic aromatic hydrocarbons, described in the state of the art as a complete carcinogen, which initiates and promotes neoplasias.

Because of these features, DMBA is unique amongst inducing drugs, being widely used studies of chemically-induced carcinogenesis, without the need for additional adjuvant substances to trigger the neoplastic process.

Animal models that use hamsters treated with DMBA solution are widely used in the study of cancer derived from precancerous lesions (dysplasias) induced by this carcinogenic product in epithelial tissues of the upper aerodigestive tract (mouth, tongue, esophagus) and. Thus, the referred experimental model is adequate to demonstrate the practical usefulness, that is, the therapeutic ability of the biological response modifier (proteic aggregate of ammonium and magnesium phospholinoleate-palmitoleate anhydride) for this situation of interest for the purposes of the present invention.

Therefore, in order to establish the practical usefulness of the compound to be used for the preventive treatment of cancer, the experiment reports the action of the biological response modifier (proteic aggregate of ammonium and magnesium phospholinoleate-palmitoleate anhydride) in an experimental model of induced carcinogenesis in Syrian golden hamsters using the hydrocarbon compound 7,12-Dimethylbenzanthracene (DMBA) to induce neoplasia in the tissues of the epithelial lining of the mouth of the animals.

The results obtained can be extrapolated to all those situations that involve the formation and evolution of precancerous lesions in the outer and inner epithelium, induced or promoted by external aggressive agents (carcinogens), such as chemical agents, solar radiation and finally precancerous cell change associated to viruses, as will be extensively explained in the present report.

The practical example that follows is for illustrative purposes only and does not intend to limit the scope of the present invention.

Example of the Practical Use of the Biological Response Modifier (Proteic Aggregate of Ammonium and Magnesium Phospholinoleate-Palmitoleate Anhydride) in an Experimental Model of Carcinogenesis by DMBA

Materials and Methods:

Animals: Syrian golden hamsters (Mesocriatus auratus) males and females aged 60 days with mean body weight between 95 and 100 grams

Inducer of carcinogenesis: 7,12-dimethilbenz (a) anthracene (DMBA, Sigma Aldrich).

Biological response modifier: Proteic aggregate of ammonium and magnesium phospholinoleate-palmitoleate anhydride

Dosage: 10 mg/kg/day per animal

Route of administration: subcutaneous

Experimental Procedure:

60 Syrian golden hamsters (Mesocriatus auratus), 30 males and 30 females aged 60 days and with a mean body weight between 90 and 100 grams were used.

The animals were distributed into six groups: three treatment groups and three control groups, with 10 animals in each group, corresponding to three experimental periods at 7, 13 and 20 week intervals.

The animals were individually identified with ear tags.

The animals were observed and weighed daily and the events, including changes in behavior and possible deaths, were recorded.

DMBA solution (0.5% in mineral oil) was applied topically to the cheek pouch of each animal using a brush, three times a week.

The biological response modifier (proteic aggregate of ammonium and magnesium phospholinoleate-palmitoleate anhydride) was applied every day in the groups treated with 10 mg/kg/subcutaneous DMBA diluted in saline. The animals in the control group were subcutaneously injected the same volume of saline alone.

At the end of each period established for each experimental group, that is, in the 7th, 14th and 20th weeks, the animals in the treatment groups and control groups (10 animals in each group) were sacrificed.

The left cheek pouch of each animal was removed, fixed in 10% buffered formaldehyde for macroscopic evaluation and preparation of slides for histological examination.

For histological examinations, the cheek pouches were included in paraffin blocks, and then 5-μm thick sections were cut using a microtome and stained with hematoxilin-eosin (H.E.) for assessment of cellular changes and comparison between the control group and the treatment groups.

Results: Although clinical manifestations were not observed on macroscopic examination for the control and treatment groups, in the 7th week the histological examination of the tissues of the cheek pouches of all the animals in the control group (DMBA) sacrificed in this period showed significant changes, with various degrees of atypia (dysplasias).

None of the animals in the treatment group sacrificed in the 7th week showed significant atypias on the histological examinations of tissue extracts of the cheek pouches of the animals.

On the 8th week, all animals in the control (DMBA), that is, 100%, showed clinical changes on macroscopic examination consisting of leukoplakia in the same region painted with the carcinogen, which was very swollen (congested). Only two animals, that is, 20% of the treatment group showed discrete macroscopic lesions compared to the control animals.

On the 14th week, the histological examination of tissue extracts of the cheek pouches of the animals in the control group (DMBA) showed microscopic changes consistent with squamous cell carcinoma, that is, 100% of the animals. Of the animals in the treatment group that were sacrificed on the 14th week, although two of them had discrete macroscopic lesions, only one had microscopic lesions in the tissue consistent with squamous cell carcinoma, that is, 10% of the animals.

On the 20th week, besides macroscopic changes consisting in leukoplakia, generalized congestion of the area painted with DMBA and signs of bleeding in isolated spots, histological examination of the tissues of cheek pouches of all animals in the control group (DBMA), that is, 100% of the animals also showed microscopic lesions consistent with squamous cell carcinoma.

Two animals in the control group (DMBA) died in the 18th and 19th weeks, that is, 20% of the animals. None of the animals in the group treated with the biological response modifier died during the experiment.

The cheek pouches of these animals that died during the 18th and 19th weeks were preserved for histopathological examination and also showed microscopic changes consistent with squamous cell carcinoma.

Of the animals in the treatment group sacrificed in the 20th week, two had macroscopic changes consisting of leukoplakia and congestion in the area painted with DMBA, though more discrete than in the control group. However, on microscopic examination, the tissue of the cheek pouch of these animals, that is, 20% of the total revealed signs and changes consistent with squamous cell carcinoma.

Discussion of the results: The use of the biological response modifier (proteic aggregate of ammonium and magnesium phospholinoleate-palmitoleate anhydride) had a remarkable protective effect against DMBA-induced carcinogenesis in the animals of this experiment compared to the animals in the control group treated with 0.9% saline.

That is, whereas 100% of the animals in the control group (DMBA) treated with NaCl (0.9%) alone had microscopic signs in the oral epithelium consistent with squamous cell carcinoma, only 20% of the animals treated with the biological response modifier (proteic aggregate of ammonium and magnesium phospholinoleate-palmitoleate anhydride) had cellular changes consistent with carcinoma in the tissue of the cheek pouch, in the group treated and followed up for the longest period of time, that is, (20) twenty weeks.

This remarkable and surprisingly effect involving the biological response modifier (proteic aggregate of ammonium and magnesium phospholinoleate-palmitoleate anhydride), that is, prevention of the occurrence and/or evolution of the dysplasia to cancer in living beings, in this case, experimental animals, was not described in the state of the art until now.

Although the mechanisms of action of this protective effect are largely hypothetical, it should be stressed that this protection begins in the early state of cell change, since the animals treated with the biological response modifier (proteic aggregate of ammonium and magnesium phospholinoleate-palmitoleate anhydride) examined in the 7th week did not show significant macroscopic and microscopic changes compared to the non-treated control animals.

One hypothesis on the mechanism of action can be related to the stimulus to toll-like cell receptors by the biological response modifier (proteic aggregate of ammonium and magnesium phospholinoleate-palmitoleate anhydride), with reports in the state of the art on the action of ligands for TLR-2 and TLR-4 and mechanisms of protection and regeneration of cellular tissue damaged by various factors. The data shown in Table T1 and Table T2 of U.S. Pat. No. 3,516,628 show the ability of the biological response modifier (proteic aggregate of ammonium and magnesium phospholinoleate-palmitoleate anhydride) of binding to TLR-2 and TLR-4 receptors.

Another hypothetical possibility is the existence of a dual mechanism of action, that is, that the compound (proteic aggregate of ammonium and magnesium phospholinoleate-palmitoleate anhydride), besides providing a protective effect that is possible due to the ability of this compound to act as a ligand (agonist) for toll-like cell receptors, which induce cell repair mechanisms, also induces or stimulates the action of the immune system on epithelial cells affected in their early precancerous stage, destroying them and, thus, ensuring a preponderance of healthy cells in the epithelial region.

Regardless of the mechanisms of action, the remarkable practical result of the experiment encourage its immediate use as a preventive therapy for situations of high risk for the development of neoplasias, that is, in the treatment of precancerous lesions, e.g. those occurring in skin carcinomas, malignant melanoma and other skin tumors, or else it can be used in the preventive treatment of uterine cancer that starts with changes in uterine epithelial cells caused by the action of various types of the human papillomavirus (HPV).

Or else, and finally for tumors of the digestive tract and/or upper airway tract (mouth, throat) derived from precancerous lesions of the lining epithelium, as shall be extensively demonstrated in the present report, with practical examples of the use of the invention. Other examples and references of the current state of the art are provided below to help understanding the remarkable and innovative properties of this invention.

Precancerous Lesions and Cervical Dysplasia and Evolution to Cancer—Associated Factors—Treatment—State of the Art

The so-called cervical dysplasia is considered a premalignant or precancerous change or modification of the epithelial cells of the cervix.

In some cases, cervical dysplasia remains stable or is eliminated by the immune system However, if untreated; it may become a chronic condition and progress to cervical cancer.

Cervical dysplasia is routinely diagnosed in clinical practice using the Papanicolaou test, also named Pap smear or Pap test, for the early detection of lesions.

The Pap smear test is a routine test where cells from the cervix (uterus) are collected for the cytological examination of the smear.

Besides its classical use for the assessment of tissue in the cervical region, the Pap test is also used for the evaluation of the effectiveness of the treatments.

The results of Pap smears are grouped in 5 (five) categories: Class I indicates absence of abnormal cells, class II usually indicates inflammation or infection and Class III indicates the presence of changed cells (dysplasia).

Class III can be subdivided into three subclasses or other types of dysplasia: mild, moderate or severe.

Cauterization is generally indicated in the mild and moderate types. In severe dysplasia, the removal of a segment of the cervix called conization or other surgical measures may be necessary.

Class IV represents a highly suspicious result of malignancy, and Class V is considered representative of a manifested neoplasia.

Determinants for Cervical Dysplasia—Chronical Infection by HPV

In the state of the art, it is well established that the determinants (causes) of most chronic cervical dysplasias is the infection by the human papilloma virus—HPV, which is sexually transmissible. There are around 100 types of HPV identified, of which one dozen are implied in the process that generates cervical dysplasia and its progression to cervical cancer.

The HPV-16 and HPV-18 types are said to be responsible for 60% of the cases of cervix cancer in the world. We cite: Munoz N, Castellsague X, de Gonzalez A B, Gissmann L. Chapter 1: HPV in the etiology of human cancer. Vaccine. 2006; 24 Suppl 3:S1-S10.)

The types HPV 6, HPV-11, HPV-16, HPV-18, HPV-33 and HPV-45 are considered to be of high risk for carcinogenesis. All these types (HPV 6, HPV-11, HPV-16, HPV-18, HPV-33, HPV-45) cited and recognized as being of high risk for carcinogenesis are associated to the development of cervical dysplasias, that is, precancerous lesions because they may evolve into cancer. The presence and characterization of these viral types are assessed after the confirmation of cervical dysplasia, through various laboratory techniques, such as in situ hybridization, hybrid capture, PCR).

There is no scientific consensus so far whether the cellular changes called dysplasias (cervical dysplasia) can be directly associated to viral activity (direct cytopathic effect) or indirectly, that is, due to cellular changes induced by the pathogen-host interaction (indirect cytopathic effect) or finally, by the combination of both situations or causes.

Nevertheless, regardless of the causal factors of cervical dysplasia it is evident for any expert with knowledge of the state of the art, without the need for further explanations, that the concomitant treatment of the primary carcinogenic cause, that is, of the chronic viral infection performed before, during or after the treatment or removal of dysplasia, which is a lesion usually associated to HPV, is an important condition for the success of the therapy, because it reduces the chances of recurrence of infection that can lead again to dysplasia.

Thus, an hypothetical ideal treatment for the precancerous cellular condition or change, that is, cervical dysplasia, which is associated to chronic infection by HPV, should fight or eliminate the changed cells (dysplasia) and at the same time control the causative agent, that is, the HPV. As will be shown in the present report, the invention has both abilities.

Cervical Dysplasia—Preventive Therapies—Treatment Options

Since chronic infection by HPV is known to be associated to the induction of dysplasias and evolution to cancer, various treatments were developed and are available in the state of the art to attempt to control and/or eliminate the viruses and associated lesions and/or at least provide a palliative treatment of dysplasias and cancer. For examples of the state of the art: Scheinfeld N, Lehman D S. An evidence-based review of medical and surgical treatments of genital warts. Dermatol Online J. 2006 Mar. 30; 12(3):5.

Infection by HPV can be described in three main forms of presentation: clinical, subclinical and latent. The clinical form, with the presence of macroscopic lesions (genital warts) in the anogenital region; the subclinical form, characterized by the presence of diffuse epithelial hyperplasia and dysplasia, seen through the colposcope and after application of contrast medium (acetic acid) and the latent form, that is, without histological changes, though with the presence of the viral DNA detected by techniques such as hybridization, hybrid capture or PCR (polymerase chain reaction).

The traditional treatments aimed mostly to the elimination of lesions associated to HPV or its clinical manifestations, such as genital warts, involve local therapies, with the use of caustic agents such as podophyllin and its derivatives and also trichloroacetic acid.

The most widely used techniques with varying degrees of success in the state of the art are: local excision, cryotherapy, CO2 laser vaporization and electrocauterization. However, the surgical treatments for the clinical and subclinical forms of HPV are not very efficient and have a high degree of recurrence. They involve long periods of time and are often painful and/or disfiguring.

Main Problems Involving Surgical Treatments for the Elimination of Dysplasias

Surgical procedures to remove HPV infected tissues can be painful and impractical for the treatment of extensive lesions. In the state of the art there is great controversy as to whether the frequent post-treatment recurrences are due to reactivation of subclinical infection or derived from normal epithelium left untreated. The most frequent complications include pain, local secretion, ulceration, infection and delayed healing. Permanent scarring may also occur. The results are conflicting in terms of recurrence and persistence of subclinical lesions.

Practical Example of the Use of Present Invention—Use of the Biological Response Modifier in Recurrent HPV Infection and Cervical Dysplasia.

Clinical study: Post-surgical application of the biological response modifier (proteic aggregate of ammonium and magnesium phospholinoleate-palmitoleate anhydride) in 20 women with medium to severe dysplasia and HPV seropositivity.

A clinical study involving 20 women with medium to severe dysplasia, confirmed by laboratory tests (III degree Papanicolaou) and HPV seropositivity (PCR method) was conducted.

All patients had previous history of routine surgical procedures for removal of the damaged tissue (electrocauterization) before the clinical trial, with recurrence of dysplasia and infection in all the cases.

The patients underwent routine examinations before the clinical trial for evaluation of dysplasia (Papanicolaou test) and cervical smears were taken for human papillomavirus (HPV) testing and typing.

Of the 20 patients that participated in the clinical trial, 10 were classified as suffering from moderate cervical dysplasia associated to HPV 11, 5 with moderate cervical dysplasia associated to HPV 18 and 5 with severe cervical dysplasia associated to types HPV-16 and HPV-18.

The results of the tests of patients on admission to the trial are shown in Table P-1

TABLE P-1
Classification of the degree of cervical dysplasia and viral
typing performed on admission to the clinical trial (basal)

| Number of | Mild | Moderate | Severe | Type of HPV |
|---|---|---|---|---|
| patients | dysplasia (**) | dysplasia | dysplasia (**) | associated (*) |
| 10 | — | 10 | — | HPV 11 |
| 5 | — | 5 | — | HPV 18 |
| 5 | — | — | 5 | HPV 16/18 |

(*) Method for detection and viral typing: PCR - Kit Amplicor (HPV) - Roche
(**) Method for detection and classification of cervical dysplasia: Papanicolaou

Periodicity: Collection of cervical smear for the test and classification of dysplasia and HPV were performed in the pretreatment period (basal) and 6 months after the end of the clinical trial.

Clinical and laboratory characterization: Patients who were previously treated with electrocauterization and showing signs of cervical dysplasia classified into medium to severe upon admission to the clinical trial.

Viral typing: All patients (100%) had at least one viral type considered high risk for developing malignancy on admission, and five patients (25%) showed association of more than one viral type (HPV 16e HPV 18).

Clinical trial design: All patients underwent again surgery, that is, electrocauterization for removal of the changed tissue (dysplasia).

They were also given a 5 mg/m2 dose of the biological response modifier intramuscularly, in physiological saline (NaCl 0.9%) in three cycles of application, as follows:

Dosage: 3 (three) cycles of 10 doses of 5 mg/m2 for all patients, injected intramuscularly, with the first dose starting 1 day after electrocauterization.

Follow-up: The patients were followed monthly by clinical examinations and colposcopy.

Final assessment: The patients were assessed again 6 (six) months or 180 days after the end of the surgical procedure associated to the applications of the biological response modifier for dysplasia and presence of HPV.

Results: The results are shown in Table P-2 and then discussed.

TABLE P-2
Classification of the degree of cervical dysplasia and viral typing
after the clinical trial (T: 180 days after the end)

| | | | Severe | |
|---|---|---|---|---|
| Number of | Mild | Moderate | dysplasia | Type of HPV |
| patients | dysplasia (**) | dysplasia (**) | (**) | associated (*) |
| 20 | — | — | — | — |
| 1 | — | — | — | HPV 18 |
| 1 | — | — | — | HPV 16/18 |

(*) Method for detection and viral typing: PCR - Kit Amplicor (HPV) - Roche
(**) Method for detection and classification of cervical dysplasia: Papanicolaou

Discussion of the results: The use of the present invention, that is, the biological response modifier associated to a surgical procedure (electrocauterization) showed remarkable results in the patients that participated in the clinical trial, acting on the cervical dysplasia and on the causative agent (HPV).

Although the sample is limited in number, it should be noted that all patients have previously undergone the same surgical procedures for removal of changed tissue (cervical dysplasia) and relapsed, that is, 100% of recurrence was observed.

The recurrence rate with the use of the invention, that is, the biological response modifier associated to surgery (electrocauterization) was 0% (zero percent), that is, none of the twenty patients showed any sign of dysplasia during assessment 180 days after the beginning of the clinical trial, although 2 (two) of them, that is, 10% of the patients still had HPV at that time (180 days after the beginning of the trial) compared to the recurrence rate of 100% for cervical dysplasia and viral presence at the beginning of the experiment reported in the same patients who received only surgical treatment.

It should be noted that these patients did not show cervical dysplasia anymore during the assessment period (180 days after the end of the clinical trial), which clearly demonstrates the direct effect of the invention on cellular change (cervical dysplasia), that is, on precancerous cells, regardless of the presence of the virus, since the laboratory tests of these patients showed the presence of residual HPV in 2 (two) patients.

This effect of the invention on the precancerous lesion (cervical dysplasia) regardless of the presence of the virus has been demonstrated, for all patients had moderate to severe dysplasia on admission to the clinical trial and after being treated with the present invention dysplasia was eliminated, though HPV was detected in a few patients (10%) (Table P-2).

Treatment of Cervical Premalignant Lesions—Conclusion

The data of Table P-1 and Table P-2 prove that the use of the claimed invention, that is the use of the immunomodulator (proteic aggregate of ammonium and magnesium phospholinoleate-palmitoleate anhydride) combined with a non-drug treatment (surgery) is able to act synergistically against premalignant lesions (cervical dysplasia, cervical metaplasia) associated with the presence of HPV.

### New Formulations for Use of the Immunomodulator for the Purposes of the Invention

According to the state of art, the immunomodulator (proteic aggregate of ammonium and magnesium phospholinoleate-palmitoleate anhydride) may be used by injectable via, that is, intramuscularly, intraperitoneally, and subcutaneously.

For these purposes, the immunomodulator (proteic aggregate of ammonium and magnesium phospholinoleate-palmitoleate anhydride) may be formulated using aqueous solutions such as sterile saline solutions for injection (Nacl 0, 9%) or also buffered solutions as pharmaceutical carriers.

In addition, some formulations for topical use of the immunomodulator are reported in the state of art (U.S. application Ser. No. 14/444,436).

Now, innovative formulations for use of the immunomodulator (proteic aggregate of ammonium and magnesium phospholinoleate-palmitoleate anhydride) for the purposes of the claimed invention, as well the methods to make such formulations are provided.

As widely known in the state of art poor water solubility of drugs and drug candidates may represent an obstacle to their use for some clinical application.

The immunomodulator (proteic aggregate of ammonium and magnesium phospholinoleate-palmitoleate anhydride) is poorly soluble in aqueous solutions. Taking in account the state of art that make possible to overcome this limitation, the use of nanonization process represents one of the feasible ways to make such compound soluble in aqueous solutions.

After several experiences to make nanoparticles using some stabilizers, such as poloxamers and chitosan, the method described below was selected for the purposes of the present invention.

### Nanoparticles and Method to Make Nanoparticles

The nanonization of crystals of the immunomodulator (proteic aggregate of ammonium and magnesium phospholinoleate-palmitoleate anhydride) was performed using the 750-Watt ultrasonic processor (Sonic and Material, Inc., Newtown-USA).

To achieve this, 1 mg of the immunomodulator (proteic aggregate of ammonium and magnesium phospholinoleate-palmitoleate anhydride) is dispersed in 5 ml of aqueous solution of 5% of a Poloxamer (Pluronic F68 or F127 at 5%—Aldrich).

Then, this dispersion was sonicated in the ultrasonic processor above mentioned in 10 cycles of 10 minutes each in an ice bath. During each cycle of sonication, the temperature of the dispersion was checked and maintained below 17° C. during the process of nanonization.

For drying the nanoparticles, they were frozen in dry ice/ethanol and lyophilized in a lyophilizer for 48 hours.

The nanoparticles of the immunomodulator (proteic aggregate of ammonium and magnesium phospholinoleate-palmitoleate anhydride) obtained by this method have a size of 480 nanometers (480 nm), while the crystals of the same immunomodulator before the nanonization had a size around 300 to 400 micrometers (300-400 um).

The nanoparticles of the immunomodulator (proteic aggregate of ammonium and magnesium phospholinoleate-palmitoleate anhydride) obtained are stable at temperatures between −4° C. to 40° C. and are 100% soluble in water and other aqueous solutions.

The nanoparticles of the immunomodulator can be used by oral, intramuscular, intraperitoneal, intravenous and subcutaneous via.

### Formulation of the Immunomodulator Using Poloxamers as Carriers

In the state of art, enhancement of the biological action of several drugs is mentioned using Poloxamers as pharmaceutical carriers.

For instance, Batrakova et at reports that incorporation of low molecular mass drugs into Pluronic micelles can increase drug solubility and drug stability, and can improve drug's pharmacokinetics and biodistribution, resulting in an enhancement of its biological properties (Batrakova E V and. Kabanov A V. Pluronic Block Copolymers Evolution of Drug Delivery Concept from Inert Nanocarriers to Biological Response Modifiers. J Control Release. 2008 Sep. 10; 130(2): 98-106.)

Starting on the state of art, the Applicant developed a new formulation of the immunomodulator (proteic aggregate of ammonium and magnesium phospholinoleate-palmitoleate anhydride) using Poloxamers as pharmaceutical carrier to be used for the purposes of the present invention, using the method described below.

### Method for Dispersion of the Immunomodulator Using Poloxamers to Provide Enhanced Biologic Effects

For this goal, 1 mg of the immunomodulator (proteic aggregate of ammonium and magnesium phospholinoleate-palmitoleate anhydride) is added to a 5 mL of aqueous solution of 5% of a Poloxamer (Pluronic F68 or F85 at 5%—Aldrich).

The composition of poloxamer plus immunomodulator (proteic aggregate of ammonium and magnesium phospholinoleate-palmitoleate anhydride) is agitated using a mixer at least 250 rpm for 5 (five) minutes.

As a result, the immunomodulator (proteic aggregate of ammonium and magnesium phospholinoleate-palmitoleate anhydride) is dispersed into the Poloxamer forming micelles and such system (immunomodulator+poloxamer) remains stable for 5 hours.

After this period, the system slowly degrades and the immunomodulator separates from the aqueous phase and finally precipitates.

This formulation of the immunomodulator (proteic aggregate of ammonium and magnesium phospholinoleate-palmitoleate anhydride) using poloxamers as pharmaceutical carriers can be used by intramuscular, intraperitoneal and subcutaneous via.

Forms of Association and/or Combination for the Various Treatments—Administration of Compounds and/or Pharmacologically Active Formulations

The active substances, that is, the biological response modifier (proteic aggregate of ammonium and magnesium phospholinoleate-palmitoleate anhydride), and the other pharmacologically active substances, for the purposes of the present invention, can be supplied for use alone or as separate parts for admixing, whenever possible, as well as in solid form solutions, in microencapsulated pharmaceuticals, in liposomes, in nanoparticles, or in separate systems for administration, and finally in injectable and oral forms.

The preparation of the simple form of administration of one of the substances of the present invention, e.g. the biological response modifier (proteic aggregate of ammonium and magnesium phospholinoleate-palmitoleate anhydride) can be made with any of the aqueous solutions known in the state of the art and additionally with excipients, suspensions, transporters and/or stabilizers known in the state of the art.

The other active substances that can be used for the purposes of the present invention, in association and/or combination with the biological response modifier (proteic aggregate of ammonium and magnesium phospholinoleate-palmitoleate anhydride) can also be applied and/or used in any way reported for each class or type, particularly those recommended by their manufacturers, or else in other ways known or reported in the state of the art.

More than one pharmacologically active substance can be used for the purposes of this invention, as long as the biological response modifier (proteic aggregate of ammonium and magnesium phospholinoleate-palmitoleate anhydride) is a fixed component of the association or combination. To ensure the fully effectiveness of the present invention, at least one other pharmacologically substance can be used in association or combination with the biological response modifier proteic aggregate of ammonium and magnesium phospholinoleate-palmitoleate anhydride), and, thus, more than one of these substances can be administered alone, sequentially or in combination.

More than one non-drug treatment can be used for the purposes of the present invention, as long as the biological response modifier (proteic aggregate of ammonium and magnesium phospholinoleate-palmitoleate anhydride) is a fixed component of the association or combination. To ensure the full effectiveness of the invention, at least one non-drug treatment can be used in association or combination with the biological response modifier (proteic aggregate of ammonium and magnesium phospholinoleate-palmitoleate anhydride), and more than one of these non-drug treatments can be used alone, sequentially or in combination.

More than one drug and non-drug treatment can be used for the purposes of the present invention, as long as the biological response modifier (proteic aggregate of ammonium and magnesium phospholinoleate-palmitoleate anhydride) is a fixed component of the therapeutic association or combination.

To ensure the full effectiveness of the invention, at least one non-drug treatment and one drug treatment can be used in association or combination with the biological response modifier (proteic aggregate of ammonium and magnesium phospholinoleate-palmitoleate anhydride) biological response modifier (proteic aggregate of ammonium and magnesium phospholinoleate-palmitoleate anhydride), and, thus, more than one of these non-drug and drug treatments can be used alone, sequentially or in combination.

The biological response modifier (proteic aggregate of ammonium and magnesium phospholinoleate-palmitoleate anhydride) is obtained through biological action exerted by the mycelium of the Aspergillus oryzae (A. oryzae) fungus, in appropriate culture medium, as described in the state of the art in PI-0305373-3, Ser. No. 10/978,683, EPA 0426250.3.2405 and U.S. Pat. No. 8,889,153 B2.

The other active substances that can be used for the purposes of the present invention, in association and/or combination with the biological response modifier (proteic aggregate of ammonium and magnesium phospholinoleate-palmitoleate anhydride) can be obtained from any production sources and methods known for every class or type described in the state of the art.

The other non-drug treatments that can be used for the purposes of the present invention, in association and/or combination with the biological response modifier (proteic aggregate of ammonium and magnesium phospholinoleate-palmitoleate anhydride) can be selected from various types and forms known in the state of the art, as long as they are related to the treatment of cancer and its complications or comorbidities.

The production method of the immunomodulator (proteic aggregate of ammonium and magnesium phospholinoleate-palmitoleate anhydride) used for the purposes of the present invention is equally contained and reported in the state of the art in PI-0305373-3 EPA 0426250.3.2405, U.S. Pat. No. 8,889,153 B2 and basically consists of the use of the Aspergillus oryzae fungus as fermentation agent acting on culture medium and in suitable conditions for the production of the cited immunomodulator, specifically selected to be used for the purposes of the present invention.

Any animal species can benefit from the present invention. Although the main purpose or intended field of application of the present invention is the treatment of human beings, the invention and its practical use cannot be limited to this species.

Without further elaboration, it is believed that one skilled in the art can, using the report, utilize the present invention to its fullest extent.

1. A compound for use in a method of treatment of cancer, including precancerous lesions, and adverse events caused by the disease or anti-cancer agents and treatments, including cancer cachexia, lymphopenia, neutropenia, febrile neutropenia, the compound comprising in combination:
(a) an immunomodulator, wherein the immunomodulator is a proteic aggregate of ammonium and magnesium phospholinoleate-palmitoleate anhydride, with molecular weight of 320.000 Dalton, having of 11.6±4.0% of total lipids, 22.7±5.0% of palmitoleic acid, 42.9±2.0% of linoleic acid, 32.0±3.0% of oxidated linoleic acid, 20.1±0.9% of magnesium ions, 10.0±3.3% of ammonium ions, 45.2±2.7% of phosphate, and 0.49±0.01% of proteins, and
(b) at least one anti-cancer agent or treatment suitable for treating the disease, said agent or treatment providing synergistic effects without additional toxicity when used with the immunomodulator wherein the anti-cancer agent is selected from the group consisting of: Alkylating agents (mechlorethamine, cyclophosphamide, ifosfamide, melphalan, chlorambucil), ethyleneimines and methylmelamines (thiotepa and hexamethylmelamine), alkyl sulphonates (busulfan), nitrosureas (carmustine and streptozocin), triazenes (dacarbazine and temozolimide), platinum complexes (cisplatin, carboplatin, oxaliplatin, nedaplatin, triplatin tetranitrate, satraplatin), folic acid antagonists (methotrexate, trimethoprim, pyrimethamine), purine analogues (azathioprine, mercaptopurine, thioguanine, fludarabine, pentostatin, cladribine), pyrimidine analogues (5-fluorouracil (5-FU), gemcitabine, floxuridin, cytarabine), mitotic inhibitors (vinblastine, vincristine, vindesine, vinorelbine), terpenoids (taxane, paclitaxel, docetaxel, larotaxel), antitumor antibiotics (daunorubicin, doxorubicin, epirubicin, idarubicin, mitoxantrone, pixantrone, valrubicin, actinomycin, bleomycin, mitomycin, plicamycin, hydroxyurea), topoisomerase inhibitors (irinotecam, topotecan, amsacrine, etoposide, etoposide phosphate, teniposide), tyrosine kinase blockers (erlotinib, gefitinib, imatinib, sunitinib, sorafenib), hormones (dexamethasone, finasteride, tamoxifen, fulvestrant, anastrozole, letrozole, exemestane, megestrol, goserelin, leuprolide, diethylstilbestrol), monoclonal antibodies (alemtuzumab, bevacizumab, cetuximab, ocrelizumab, ofatumumab, panitumumab, rituximab, trastuzumab) monoclonal antibodies conjugated with radioactive particles (ibritumomab tiuxetan, tositumomab), angiogenesis inhibitors (bevacizumab), stimulation factors of the myeloid lineage (filgrastim, pegifigrastim, lenograstim, molgramostim, sargramostim) cytokines (interleukin-2 (IL-2), interleukin 12(IL-12), interferon alpha, interferon alpha-2a, peginterferon alpha-2a, interferon alpha-2b, peginterferon alpha-2b, interferon alpha-n1, interferon alphacon-1, interferon beta, interferon beta-1a, interferon beta-1b, tumor necrosis factor (TNF), immunotherapeutic drugs (BCG vaccine and derivatives, levamisole, isoprinosine), biological response modifiers (imiquimod and resiquimod), therapeutic vaccines for cancer (autoimmune vaccines, dendritic cell vaccines), enzymes (L-asparaginase), polyunsaturated fatty acids or Pufas (eicosaminopentoic acid-EPA, linoleic acid docosahexanenoic acid-DHA), aminoacids (arginine, glutamine), steroids (megestrol acetate), steroidal anti-inflammatory drugs (hydrocortisone, cortisone, corticosterone, dexamethasone, betamethasone, prednisolone, prednisone, methylprednisolone, budesonide, beclomethasone), derivatives of tetrahydrocannabinol (dronabinol), non-steroidal anti-inflammatory drugs (ibuprofen), enzyme inhibitors (eicosapentaenoic acid, hydrazine sulfate), hormones (melatonin, somatropin), antacids (aluminum hydroxide and magnesium hydroxide and others), H2 blockers (famotidine, cimetidine, ranitidine and others), proton pump inhibitors (omeprazol, esomeprazol, lansoprazol, pantoprazol, rabeprazol), prokinetics (metoclopramide, domperidone, cisapride and others), mucosal protectors (sucralfate), and combinations thereof,
wherein the anti-cancer treatment is selected from the group consisting of: surgical procedures (surgery, cryosurgery, electrocauterization, surgery associated to polarized light or laser with the use of photosensitizing substances, removal of lesions by chemical abrasion, removal of lesions by electrocauterization, endoscopic ablation, endoscopic radiofrequency ablation with the use of balloon catheter) transplantation of bone marrow cells, systemic and localized radiotherapy, and combinations thereof.

2. The compound according to claim 1, wherein the at least one anti-cancer agent is selected from the group consisting of: alkylating agents, ethyleneimines and methylmelamines, alkyl sulphonates, triazenes, platinum complexes, folic acid antagonists, purine analogues, pyrimidine analogues, mitotic inhibitors, terpenoids, antitumor antibiotics, topoisomerase inhibitors, tyrosine kinase blockers, hormones, monoclonal antibodies, monoclonal antibodies conjugated with radioactive particles, angiogenesis inhibitors, stimulation factors of the myeloid lineage, cytokines, immunotherapeutic drugs, biological response modifiers, therapeutic vaccines for cancer, enzymes, polyunsaturated fatty acids, aminoacids, steroids, steroidal anti-inflammatory drugs, non-steroidal anti-inflammatory drugs, derivatives of tetrahydrocannabinol, hormones, antacids H2 blockers proton pump inhibitors, prokinetics, mucosal protectors and combinations thereof.

3. The compound according to claim 1, wherein the at least one anti-cancer treatment is selected from the group consisting of: surgical procedures, transplantation of bone marrow cells, systemic and localized radiotherapy.

4. The compound according to claim 1, wherein the amino acid content in the proteic aggregate is: Asp 7.19%, Thr 3.56%, Ser 7.56%, Glu 8.53%, Pro 0.5%, Gly 9.69%, Ala 7.46%, Val 1.0%, Met 4.38%, Isoleu 2.54%, Leu 3.03%, Tyr 0.5%, Phe 1.0%, His 2.83%, Lys 3.56%, Trp 1.3%, and Arg 35.2%.

5. The compound according to claim 1, wherein the cancer tumours are select from the group consisting of: solid tumors, non-solid tumours, internal tumours and external tumours.

6. The compound for use according to claim 1, wherein the precancerous oral lesions are selected from the group consisting of oral dysplasia, oral metaplasia.

7. The compound for use according to claim 1, wherein the precancerous cervical lesions are selected from the group consisting of: cervical dysplasia, cervical metaplasia.

8. The compound for use according to claim 1, wherein the cancer cachexia is selected from the group consisting of: primary cancer cachexia and secondary cancer cachexia.

9. The compound for use according to claim 1, wherein the hematologic adverse events are selected from the group consisting of: lymphopenia, neutropenia, and febrile neutropenia.

10. A method of treating cancer, the method comprising administering a therapeutically effective amount of the compound of claim 1 to a subject in need thereof, wherein the anti-cancer agent or treatment is suitable for treating the disease.

11. The method according to claim 10, wherein the immunomodulator and the anti-cancer agents or treatments to be associated to the immunomodulator, is performed sequentially, simultaneously, or consecutively, in a procedure judged effective against the disease.

12. The method according to claim 10, wherein the anti-cancer agent or treatment is suitable for treating the cancer lesion.

13. A method of treating cancer cachexia, the method comprising administering to a subject in need thereof an effective amount of the compound according to claim 1.

14. A method of treating hematologic adverse events, including lymphopenia, neutropenia and febrile neutropenia, the method comprising administering to a subject in need thereof an effective amount of the compound according to claim 1.

15. A method of treating an immunodeficiency of a host caused by cancer, the method comprising administering to a subject in need thereof an effective amount of the compound according to claim 1.

16. A method of treating an immunodeficiency of a host caused by anti-cancer drugs, the method comprising administering to a subject in need thereof an effective amount of the compound according to claim 1.

17. A method of treating an immunodeficiency of a host caused by anti-cancer treatments, the method comprising administering to a subject in need thereof an effective amount of the compound according to claim 1.

18. A method to improve the quality of life of patients of cancer, the method comprising administering to a subject in need thereof an effective amount of the compound according to claim 1.

19. The compound according to claim 1, wherein said synergistic effects are selected from the group consisting of potentiating therapeutic effects, increasing the time period of therapeutic effects, using smaller doses of anti-cancer agents, using higher doses of anti-cancer agents, recovering the effectiveness of the immune system, recovering of primary and secondary cachexia, recovering of neutropenia, febrile neutropenia and lymphopenia and a shorter period of treatment.

20. A pharmaceutical composition comprising the compound of claim 1 and a pharmaceutically acceptable carrier.

21. The pharmaceutically acceptable carrier according to claim 20 comprising aqueous solutions, buffered saline solutions and poloxamers

21. (canceled)

22. The pharmaceutical composition according to claim 21, wherein the composition is a preparation selected from the group consisting of: an aqueous solution, a solid form solution, a microencapsulation, nanoformulations and micelles

22. (canceled)

23. Use of proteic aggregate of ammonium and magnesium phospholinoleate-palmitoleate anhydride, as a fixed component for the manufacture of new drugs with at least one other pharmacologically active compound or substance select among the compounds of claim 2.

24. The pharmaceutical composition according to claim 21, further comprising a component selected from the group consisting of: an excipient, a suspension, a transporter, a stabilizers and combinations thereof.

25. The pharmaceutical composition according to claim 21, wherein the composition is present in an injectable form, or is present in an oral form.
